(19) United States
(12) Patent Application Publication
(10) Pub. No.: US 2015/0337030 A1
(43) Pub. Date: Nov. 26, 2015

(54) METHODS TO TREAT ALZHEIMER'S DISEASE USING APOE INHIBITORS

(71) Applicant: THE TRUSTEES OF COLUMBIA UNIVERSITY IN THE CITY OF NEW YORK, New York, NY (US)

(72) Inventor: Asa ABELIOVICH, New York, NY (US)

(21) Appl. No.: 14/404,762

(22) PCT Filed: May 31, 2013
(86) PCT No.: PCT/US13/43738

§ 371 Date: Dec. 1, 2014

Related U.S. Application Data
(60) Provisional application No. 61/653,710, filed on May 31, 2012.

(51) Int. Cl. C07K 16/18; C12N 15/113

(52) U.S. Cl. CPC C07K 16/18; C12N 15/113; C12N 2320/34; C12N 2310/11; C12N 2310/14

(57) ABSTRACT

Non-familial late-onset Alzheimer's disease (LOAD), a condition associated with the accumulation of the amyloid precursor protein-derived (APP) Abeta fragment in the brain, can be the consequence of combined genetic and environmental risk factors. One of these risk factors is the presence of the apolipoprotein E4 (APOE4) allele. This invention provides for a neuron model that can be used to screen and identify compounds that can prevent the APOE4-induced pre-LOAD state. This invention provides for methods for the treatment and/or prevention of a neurodegenerative disorder by using an inhibitor of APOE4, such as an antibody inhibitor, or by using an excess of APOE3 protein.

This application claims priority to U.S. Provisional Application No. 61/653,710, filed May 31, 2012, which is incorporated herein by reference in its entirety.

All patents, patent applications and publications, and non-patent publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Neurodegenerative diseases are a major public health concern. The increasing number of patients with neurodegenerative diseases imposes a major financial burden on health systems around the world.

Alzheimer disease (AD) is a neurodegenerative dementing disorder of late onset characterized by progressive neuronal loss, especially in the cortex and the hippocampus (Goedert M, Spillantini M G (2006) 314:777-781). The two main histopathological hallmarks of AD are the accumulation of extracellular neuritic plaques, consisting predominantly of β-amyloid (Aβ), and of neurofibrillary tangles, consisting mainly of hyperphosphorylated forms of the microtubule-associated protein tau (Goedert M, Spillantini M G (2006) Science 314:777-781). The majority of AD is sporadic (SAD), but variants in apolipoprotein E (ApoE) (Goedert M, Spillantini M G (2006) Science 314:777-781) and in SORL1, a neuronal sorting receptor (Rogaeva et al., (2007) Nature Genet. 39:168-177), can be predisposing genetic factors. At least three genes have been identified in the familial form (FAD): amyloid precursor protein (APP), presenilin-1 (PS1), and presenilin-2 (PS2).

More than half of the patients with dementia have Alzheimer's disease (AD). The prevalence for AD between the age 60-69 years is 0.3%, 3.2% between that age 70-79 years, and 10.8% between 80-89 years of age (Rocca, Hofman et al. 1991). Survival time after the onset of AD is in the range of 5 to 1 2 years (Friedland, 1993).

APOE4 is the major known genetic risk factor for late-onset AD (see Huang, 2010). APOE4 plays both Aβ-dependent and Aβ-independent roles (see Huang, 2010). APOE knockout and knockin transgenic animals have been previously described (see Kim et al., 2009). Multiple and conflicting mechanisms exist for the role of APOE4 role in AD pathogenesis. Also, few human neuronal model systems are available for the mechanistic analysis of late-onset Alzheimer's Disease (LOAD). Despite advances in the treatment of AD, there remains a need for improved therapeutics of AD and for methods to identify compounds suitable for the treatment, prevention or inhibition of AD.

### SUMMARY OF THE INVENTION

The present invention relates to the discovery of an effect of a major Alzheimer's Disease risk allele, APOE4, on APP processing and APP endosomal trafficking. The invention further relates to the discovery that an antibody selective for APOE4, or an excess amount of APOE3 protein, prevents these defects. This invention provides for methods for the treatment and/or prevention of a neurodegenerative disorder by using an inhibitor of APOE4, such as an antibody inhibitor, or by using an excess of APOE3 protein. This invention provides for a human neuron model of an APOE4-induced pre-LOAD state.

The present disclosure provides methods for the treatment and/or prevention of Alzheimer's Disease. In one aspect, the disclosure provides a method of treating, preventing or delaying the onset of sporadic Alzheimer's Disease in a subject, the method comprising administering to the subject a therapeutically effective amount of an antibody that binds to the APOE4 protein, thereby treating or preventing the Alzheimer's Disease.

In another aspect, the disclosure provides a method of treating, preventing or delaying the onset of sporadic Alzheimer's Disease in a subject, the method comprising administering to the subject a therapeutically effective amount of a peptide or a peptidomimetic that binds to the APOE4 protein, thereby treating or preventing the Alzheimer's Disease.

In another aspect, the disclosure provides a method of treating, preventing or delaying the onset of sporadic Alzheimer's Disease in a subject, the method comprising administering to the subject a therapeutically effective amount of an antisense RNA, or a siRNA, that inhibits expression of the gene that encodes the APOE4 protein, thereby treating or preventing the Alzheimer's Disease.

In one embodiment, the subject is heterozygous for the APOE4 allele. In another embodiment, the subject is homozygous for the APOE4 allele. In one embodiment, an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

In one embodiment, the antibody is a monoclonal antibody or a polyclonal antibody. In another embodiment, the APOE4 protein comprises SEQ ID NO:1.

In one embodiment, the antisense RNA or the siRNA binds to a human nucleic acid sequence comprising SEQ ID NO: 2.

In one embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the antibody. In another embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the peptide or peptidomimetic. In another embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the antisense RNA or the siRNA. In one embodiment, the levels of Aβ40, Aβ42, and sAPPβ in the subject are measured in the cerebro-spinal fluid of the subject.

In another aspect, the disclosure provides a method of treating, preventing or delaying the onset of sporadic Alzheimer's Disease in a subject, the method comprising administering to the subject a therapeutically effective amount of APOE3 protein, thereby treating or preventing the Alzheimer's Disease.

In one embodiment, the subject is heterozygous for the APOE4 allele. In another embodiment, the subject is homozygous for the APOE4 allele. In one embodiment, an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

In one embodiment, the APOE3 protein is delivered to a cell of the subject through viral-mediated delivery. In one embodiment, the APOE3 protein comprises SEQ ID NO:3.

In one embodiment, the treating or preventing comprises reducing the levels of Aβ40, Aβ42, and sAPPβ, or any combination thereof, in the subject, as compared to the levels of Aβ40, Aβ42, and sAPPβ in the subject prior to administration of the APOE3 protein. In another embodiment, the levels of Aβ40, Aβ42, and sAPPβ in the subject are measured in the cerebro-spinal fluid of the subject.

In another aspect, the disclosure provides a method of treating, preventing or delaying the onset of sporadic Alzheimer's Disease in a subject, the method comprising administering to the subject a therapeutically effective amount of an antibody that binds to the APOE2, APOE3 or APOE4 protein, or any combination thereof, thereby treating or preventing the Alzheimer's Disease.

In one embodiment, the subject is heterozygous for the APOE4 allele or the APOE2 allele. In another embodiment, the subject is homozygous for the APOE4 allele or the APOE2 allele. In one embodiment, the APOE4 protein comprises SEQ ID NO:1. In another embodiment, the APOE3 protein comprises SEQ ID NO:3.

In one embodiment, an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

In one embodiment, the antibody is a monoclonal antibody or a polyclonal antibody.

In one embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the antibody. In another embodiment, the levels of Aβ40, Aβ42, and sAPPβ in the subject are measured in the cerebro-spinal fluid of the subject.

### BRIEF DESCRIPTION OF THE DRAWINGS

FIGS. 1A-K show that hiN cells display a forebrain glutamatergic neuron phenotype with similar conversion efficiencies across all the lines. FIG. 1A-D, hiN cells (from the E3/3 culture AG07926) stained positively for the pan-neuronal markers Taul (red, in A), Tuj1 (green, in B), MAP2 (green, in D), and the neuronal nuclear marker NeuN (red, in D). FIG. 1C, represents a merged image of (A) and (B). FIG. 1E, hiN cells expressed the forebrain neuron nuclear marker Tbr1 (in red) along with MAP2 (in green). FIG. 1F, A majority of MAP2-positive (in red) hiN cells expressed the glutamatergic neuron marker vGLUT1 (in green, quantified in FIG. 1B). Inset shows magnified view of the boxed region; arrows indicate the typical vGLUT1-positive punctate pattern (see arrows in the inset). FIG. 1G, hiN cells co-cultured with rat astrocytes display punctate staining for synaptophysin, a marker for presynaptic structures, along neurite processes (18). Cells were co-stained with antibodies for Tbr1 (in red), synaptophysin (SYP; in green; see arrows in the inset), and MAP2 (in blue). Inset shows the magnified view of the boxed region. Scale bars: (FIGS. 1A-C, F and the inset of FIG. 1G) 10 μm; (D and E) 15 μm; (G) 20 μm; the inset in (F) 2.5 μm. FIG. 1H, Schematic of APP processing by β- and γ-secretases. FIG. 1I, Total absolute extracellular Aβ levels (Aβ40 [white bars]+Aβ42 [grey bars]) are presented for the panel of hiN cultures as labeled. Total Aβ was significantly increased in the context of E3/4 hiN cell cultures compared with unaffected individuals (UND) E3/3 cultures, regardless of UND or LOAD status. The UND and LOAD E3/4 cultures did not differ significantly. Media from hiN cell cultures (at 3 weeks post-transduction) or fibroblast cultures, as indicated, was assayed for secreted Aβ40 and Aβ42 by sandwich ELISA. n=6-12 per culture; error bars are SEM; *, P<0.05 for all comparisons. FIG. 1J, Quantification of total intracellular APP using sandwich ELISA. APP was enriched in hiN cell cultures relative to fibroblast precursors. However, among the hiN cultures, the UND E3/3, UND E3/4, and LOAD E3/4 groups did not differ significantly. Results represent the means±SEM (n=6-9 wells per group). *, P<0.05 for all comparisons. FIG. 1K, Accumulation of sAPPβ in the media of hiN cell cultures, as determined by sandwich ELISA. Results represent the means±SEM (n=6-12 wells per group). sAPPβ was significantly increased in the UND E3/4 and LOAD E3/4 groups relative to the UND E3/3 cultures.

FIGS. 2A-S show that APP is enriched within modified endocytic compartment puncta in APOE3/4 allele hiN cells. FIG. 2A, Immunostaining of hiN cells from representative UND (E3/3, STC0022; left), UND (E3/4, T-4560; center) and LOAD (E3/4, STC0033; right) cultures with an antibody to the APP amino-terminus labels punctate structures typical of endocytic compartment vesicles. Insets show high magnification views for visualization of APP-positive puncta (arrows). Scale bar is 5 μm. FIG. 2B, Quantification of APP-positive total puncta area (μm²; number of puncta per cell multiplied by the average puncta area) in individual UND and LOAD hiN cell cultures as labeled. Total APP-positive puncta area is significantly increased in E3/4 UND or LOAD hiN cultures, relative to E3/3 allele UND cultures, as a consequence of increased puncta number (see FIG. 6A). Results represent mean±SEM (n=12-38 cells in a total of 6 wells per group). *P<0.05. FIGS. 2C, D, Colocalization of APP-positive puncta with the early endosomal marker EEA1 in E3/3 and E3/4 hiN cells. APP-positive puncta (in red) appeared partially co-localized with EEA1 (in green), and this is most prominent in E3/4 allele (D) relative to E3/3 (C) cultures. Colocalization is visualized as yellow in the merged images. Inset panels present merged as well as individual staining patterns, for visualization of areas as demarcated by a blue square. FIG. 2K, Quantification of APP and EEA1 colocalization by fluorescent microscopy as in FIG. 2D. Puncta are defined here as distinct signal intensities 0.1 to 1 μm in diameter using Image J analysis software (NIH). FIGS. 2E, F, Colocalization of APP-positive puncta with the late endosomal/lysosome marker LAMP2 in UND and LOAD hiN cells. APP-positive puncta (in red) appeared partially co-localized with LAMP2 (in green), and this is most prominent in E3/4 allele (F) relative to E3/3 (E) cultures. Colocalization is visualized as yellow in the merged images. Inset panels present merged as well as individual staining patterns, for visualization of areas as demarcated by a blue square. FIG. 2L, Quantification of APP and LAMP2 colocalization by fluorescent microscopy as in FIG. 2F. FIGS. 2G, H, M, A subset of APP-positive puncta is co-stained with a plasma membrane dye marker at the cell periphery (CellMask™; in green). In contrast to EEA1 co-staining, peripheral plasma membrane marker co-staining appears reduced in the E3/4 hiN cells (H) relative to E3/3 hiN cells (G). Insets are high-magnification views of areas demarcated by blue squares. Quantification of colocalization by fluorescent microscopy is shown in (M). FIGS. 2O, P, Colocalization of APP-positive puncta with the BACE1 in E3/3 and E3/4 hiN cells. APP-positive puncta (in red) appeared partially co-localized with BACE1 (in green), and this is most prominent in E3/4 allele (P) relative to E3/3 (O) cultures. Colocalization is visualized as yellow in the merged images. Inset panels present merged as well as individual staining patterns, for visualization of areas as demarcated by a blue square. FIG. 2Q, Quantification of APP and BACE1 colocalization by fluorescent microscopy as in (P). FIGS. 2R, S, Quantification of EEA1 and LAMP2-positive puncta area (μm²; number of puncta per cell X average puncta area) in individual E3/3 and E3/4 hiN cell cultures as labeled. Total EEA1 (R) and LAMP2 (S)-positive puncta area is significantly increased in E3/4 UND hiN cultures, relative to E3/3 allele UND cultures, as a consequence of increased puncta number (see FIG. 6C-F) Results represent mean±SEM (n=12-38 cells in a total of 6 wells per group). *P<0.05.

FIGS. 3A-R show that altered APP compartmentalization in E3/4 hiN cells is rescued by extracellular APOE4 inhibition. FIGS. 3A, B, Quantification of Aβ40 (A) and sAPPβ (B) in media from hiN cultures treated with recombinant human APOE3 or APOE4 protein (rAPOE; 100 μg/ml, 48 hr) or an antibody specific to APOE4 (2 μg/ml; versus preimmune IgG). Results represent the means±SEM as determined in E3/3 UND (A, B; STC0022), E3/4 UND (A; AG07627, B; T-4560) and E3/4 LOAD (A, B; STC0033) hiN cultures (n=3-6 wells per line). *P<0.05. FIGS. 3C-G, Rescue of the APP endocytic phenotype in E3/4 hiN cells. E3/4 UND (T-4560) hiNs were treated with anti-APOE4 mouse IgG (D; 2 μg/ml) or pre-immune mouse IgG (C) for 48 hr, and vehicle (E), rAPOE3 (F) or rAPOE4 (G) (100 μg/ml, 48 hr) and then fixed and stained with an antibody to the APP amino-terminus. Insets show high magnification views for visualization of APP-positive puncta. FIG. 3H, Quantification of total APP-positive puncta area revealed that either rAPOE3 or anti-APOE4 antibody treatment led to a significant decrease in APP-positive puncta area within E3/4 allele UND cultures (relative to preimmune mouse IgG) that is reminiscent of the untreated E3/4 hiN cell phenotype. Results represent the mean±SEM (n=35-50 cells in 3 independent wells per group). *P<0.05. FIGS. 3I-K, Increased APP-positive puncta area in E3/3 hiN cells treated with rAPOE4. E3/3 UND (AG07926) hiN cells were treated with either vehicle (I), rAPOE3 (J) or rAPOE4 (K; 100 μg/ml) for 48 hours and then fixed and stained with an antibody to the APP amino-terminus. FIG. 3L, Quantification of total cell APP-positive puncta area revealed that rAPOE4 treatment led to a significant increase in APP-positive puncta area within E3/3 UND cultures that is reminiscent of the untreated E3/4 allele hiN cell phenotype. Results represent mean±SEM (n=35-50 cells in 3 independent wells). *P<0.05. FIGS. 3M-P, Effect of the γ-secretase inhibitor DAPT on APP-positive puncta in E3/3 and E3/4 hiN cells. E3/3 UND (upper: AG07926) or E3/4 UND (lower; T-4560) hiN cells were treated with either vehicle (left panel) or DAPT (right panel; 1 μM) for 48 hours and then fixed and stained with an antibody to the APP amino-terminus. FIGS. 3Q, R, Quantification of total cell APP-positive puncta number (Q) and average diameter (R). Results represent mean±SEM (n=25-40 cells in 3 independent wells. *P<0.05.

FIGS. 4A-N show accelerated receptor-mediated endocytosis in E3/4 hiN cell cultures. UND E3/3 (A-D; AG07926), UND E3/4 (E-H, T-4560) and UND E3/4+anti-APOE4 (I-L, T4560+α-APOE4) hiN cell cultures, were incubated with Alexa 488-conjugated transferrin (Tfh, 50 μg/ml) at 37° C. at 0° C. (on ice) for 90 minutes. Cells were then either immediately fixed (0 min; A, E and I), or cultured at 37° C. for 5 min (B, F and J), 15 min (C, G and K) or 60 minutes (D, H and I) prior to fixation. Confocal images presented are of hiN cells stained with an antibody to the early endosome marker EEA1 (red) or with CellMask plasma membrane dye (blue). Tfn fluorescence is shown in green. FIG. 4M, Quantitative analyses of the % of Tfn that is internalized, defined here as Alexa 488-Transferrin fluorescence that is internal to the cell surface (as marked with the CellMask membrane dye), presented in terms of the % of total cellular staining FIG. 4N, Quantitative analyses of Tfn colocalization with EEA1, as a % of total cellular Tfn staining. All results represent the mean±SEM; n=30 cells in 3 independent wells per group).

FIGS. 5A-N show the analysis of hiN cells from LOAD and unaffected individuals. Related to FIG. 1. FIGS. 5A, B, Quantification of MAP2-(A) and vGLUT1-(B) positive cells in a panel of hiN cell cultures derived from nine human fibroblast cultures. Each fibroblast culture was transduced with a set of four neuronal conversion factors (polycistronic ABZ plus M) (1) and subsequently cultured for 2 weeks. FIG. 5A, the percent of cells that are MAP2-positive and display extended processes (at least 3-fold greater than the soma diameter, as in FIG. 1D-G) FIG. 5B, the percent of MAP2-positive cells that stain for the glutamatergic neuron marker vGLUT1 as in (FIG. 1F). n=3 wells for each group; data are presented as mean±SEM. FIGS. 5C-E, Flow cytometric analysis of hiN cell cultures at 3 weeks after 4-factor transduction (D, E) or untransduced fibroblasts (C) stained with an antibody specific for human NCAM (E) or without a primary antibody (D). A population of NCAM-positive cells is apparent only in the stained hiN cell cultures (in purple). FIG. 5F, Percent of hiN cells (2 weeks after viral transduction) sorted from each converted cell lines using NCAM antibody. FIG. 5G, Neuronal gene expression profiles for NCAM, MAP2, GRBBR2, HTR2A and SNAP25 in UND E3/3 hiN, UND E3/4 hiN and LOAD E3/4 hiN cultures. Samples were collected 2 weeks after 4-factor transduction as indicated. All analyses were performed by quantitative real-time RT-PCR. All data were normalized to GAPDH expression. In each gene expression analysis, the gene expression in hiN was also compared to that in UND E3/3 fibroblasts so that the expression in UND E3/3 fibroblasts was normalized to 1. *p<0.05; **p<0.01; n=9. Results represent the mean±SEM. FIG. 5H, Released of Total APOE and APOE4 in the media of neuronal hiN cell cultures, as determined by sandwich ELISA. Results represent the means±SEM (n=6-12 wells per group). FIG. 5I, The Aβ42: Aβ40 ratio is not modified in UND E3/4 hiN cell cultures relative to LOAD E3/4 hiN cell cultures or UND E3/3 hiN cell cultures or the parental fibroblasts. Media from hiN cell cultures (at 2 weeks post-transduction) or fibroblast cultures, as indicated, was assayed for secreted Aβ40 and Aβ42 by sandwich ELISA. n=6-12 independent wells per culture; error bars are SEM; *, P<0.05 for all comparisons. FIGS. 5J-N, APP and BACE1 expression is not changed in each hiN cell lines. Samples were used 2-3 weeks after infected 4 factors. FIGS. 5J, K, Quantification of APP (J) and BACE1 (K) expression by fluorescence intensity. FIGS. 5L-N, Western blots analysis of APP and BACE1 in hiN cell (UND E3/3; STC0022, UND E3/4; AG07619 and LOAD E3/4; AG06263). All data were normalized to actin expression in each line. And the level of protein expression in hiN cells was compared to UND E3/3.

FIGS. 6A-F show altered APP-positive endocytic morphology in LOAD hiN cells. Related to FIG. 2. FIGS. 6A, B, Determination of APP-positive puncta number (A) and diameter (B) in each UND and LOAD hiN cell culture as labeled. Puncta diameter was quantified by Image J software; see Methods below. Results represent mean±SEM (n=12-38 cells in a total of 6 wells per group). Data in FIG. 2A are derived by the formula (Total puncta area/cell)=(number of puncta/cell)×π(mean puncta diameter/2)². FIGS. 6C-F, Determination of EEA1- and LAMP2-positive puncta number (C, D) and diameter (E, F) in each UNDs and LOAD hiN cell culture as labeled. Puncta diameter was quantified by Image J software; see Methods for details. Results represent mean±SEM (n=12-38 cells in a total of 6 wells per group). Data in FIGS. 2R, S are derived by the formula (Total puncta area/cell)=(number of puncta/cell)×π(mean puncta diameter/2)².

FIGS. 7A-H show the effect of human recombinant APOE or anti-APOE4 antibody treatment on Aβ42 production in hiN cell cultures. Related to FIG. 3. FIG. 7A, Quantification of Aβ42 in media from cultures treated with hAPOE3, rAPOE4 and anti-APOE4 antibody. Results represent the means±SEM (n=3-6 per group). *P<0.05. FIGS. 7B-E, Determination of APP-positive puncta number and diameter in vehicle, rAPOE3 and rAPOE4 treatment in APOE3 allele UND hiN cell culture (AG07926; B, C), and in vehicle and rAPOE3 or pre-immune mouse IgG and anti-APOE4 mouse IgG treatment in APOE4 allele UND hiN cell culture (T-4560; D, E). Quantification of APP-positive puncta number per cell (B, D) and diameter (C, E) under the rAPOE3, rAPOE4 (50 μg/ml) or anti-APOE4 antibody (2 μg/ml) treatment condition. Results represent the means±SEM (n=35-50 cells in 3 independent wells). *P<0.05. FIG. 7F, Determination of APP-positive puncta area in DAPT treatment in APOE3 allele UND hiN cell culture (AG07926) and APOE4 allele UND hiN cell culture (T-4560). FIGS. 7G, H, Quantification of Aβ42 (G) and Aβ42 (H) in media from cultures treated with DAPT. Results represent the means±SEM (n=3-6 per group). *P<0.05.

FIGS. 8A-N show accelerated receptor-mediated endocytosis in E3/4 hiN cell cultures. Related to FIG. 4. FIG. 8A, Schematic of the endocytic, degradation and recycling pathways for transferrin in mammalian cells. Upon binding to Tfh, the Tfn receptor (Tfr) is internalized within clathrin-coated pits by clathrin-dependent receptor mediated endocytosis. From there, the majority of Tfn is recycled to the cell surface through A Rab11-dependent vesicular pathway. A small population of receptors is typically also transported from early endosomes to lysosomes through a late endosomal pathway for degradation. FIG. 8B, Experimental designs for Tfn endocytosis analyses. FIGS. 8C-J, Confocal images of cells stained with an antibody for EEA1 (red) or with CellMask dye (blue). Tfn fluorescence is shown in green. E3/3 hiN cultures treated with APOE4 protein (C-F, AG07926+APOE4) or with APOE3 protein (G-J, T-4560+APOE3) were incubated with Alexa 488-conjugated transferrin (Tfn, 50 μg/ml) at 37° C. at 0° C. (on ice) for 90 minutes. Cells were then either immediately fixed (0 min; C, G), or cultured at 37° C. for 5 min (D, H), 15 min (E, I) or 60 minutes (F, J) prior to fixation. FIG. 8K, Quantitative analyses of the % of Tfn that is internalized, defined here as Alexa 488-Transferrin fluorescence that is internal to the cell surface (as marked with the CellMask membrane dye), and presented in terms of the % of total cellular staining. All results represent the mean±SEM (n=30 cells in 3 independent wells per group). This graph includes data from FIG. 4M for comparison. FIG. 8L, Quantitative analyses of Tfn colocalization with EEA1, as a % of total cellular Tfn staining. All results represent the mean±SEM; n=30 cells in 3 independent wells per group). This graph includes data from FIG. 4N for comparison. FIG. 8M, Quantitative analyses of Tfn colocalization with plasma membrane (marked by the CellMask dye), as a % of total cellular Tfn staining Note that these are the same data as presented in FIG. 8K above, replotted for illustration purposes. All results represent the mean±SEM; n=30 cells in 3 independent wells per group). FIG. 8N, Quantitative analyses of transferrin bound to the plasma membrane at time 0. There is no significant difference between the groups. All results represent the mean±SEM (n=30 cells in 3 independent wells per group).

FIGS. 9A-F show CI-MPR localization in hiN cells. FIGS. 9A, B, Immunocytochemical analysis of CI-MPR (in green) and LAMP2 (in red) colocalization in hiN cells from E3/3 (A; AG07926) or E3/4 (B; T-4560) cultures. FIG. 9C, Quantitative analyses of the co-localization of CI-MPR and LAMP2 shows no significant difference between the groups. FIGS. 9D, E, Immunocytochemical analyses of CI-MPR (in green) and TGN46 (in red) colocalization. FIG. 9F, Quantitative analyses of the co-localization of CI-MPR and TGN46 shows no significant difference between the groups. All the results represent the mean±SEM (n=25 cells in 3-6 independent wells per group). Scale bars: (A, B) and (D, E), 4 μm.

### DETAILED DESCRIPTION OF THE INVENTION

### Abbreviations and Definitions

The abbreviation “APOE” refers to the apolipoprotein E gene. The gene comes in three variants that encode proteins, APOE2, APOE3 and APOE4 (see O'Brien and Wong, 2011, Annu Rev Neurosci., 34: 185-204, which is incorporated by reference herein). The nucleic acid sequences of the genes encoding the different protein isoforms of APOE, including, but not limited to, the nucleic acid sequences of the open reading frames of the genes, are known in the art. The nucleic acid sequences of the genes encoding the different protein isoforms of human APOE, including, but not limited to, the nucleic acid sequences of the open reading frames of the genes, are known in the art. The amino acid sequences of APOE polypeptides and proteins, including, but not limited to, the amino acid sequences of the human APOE polypeptides and proteins, including the processed forms of the proteins and the precursor proteins, are known in the art. Reference herein to the APOE2 protein, APOE3 protein or APOE4 protein encompasses reference to either the processed form of the protein, or the precursor protein, or both.

The abbreviation “hiN” refers to human induced neuron. For additional information on hiN cells, see e.g., Qiang et al., 2011, Directed conversion of Alzheimer's disease patient skin fibroblasts into functional neurons, Cell, 146(3):359-71, which is incorporated herein by reference in its entirety.

The singular forms “a”, “an” and “the” include plural reference unless the context clearly dictates otherwise.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Description

### Alzheimer's Disease

Neurodegenerative disorders of aging are characterized by a progressive loss of neurons and their synaptic connections, and therefore regenerative cell-based approaches are particularly attractive.

Alzheimer's disease (AD) is a neurodegenerative dementing disorder of late onset characterized by progressive neuronal loss, especially in the cortex and the hippocampus (Goedert and Spillantini, Science 314:777-781 (2006)). AD patients typically present with age-associated cognitive dysfunction in multiple realms, including reduced short-term (episodic) memory and spatial disorientation, associated with neuronal and synaptic loss that is most prominent within the medial temporal lobe of the cerebral cortex and the hippocampus formation (Alzheimer, 1907; Mucke, 2009). Additional pathological hallmarks that typify AD include extracellular amyloid plaques, composed largely of Aβ fragment of amyloid precursor protein (APP), and neuronal tangles that are structured of Tau paired helical filaments (Duyckaerts et al., 2009). As the disease process is thought to extend over decades, such pathological findings on autopsy brain tissue cannot illuminate mechanisms of disease onset.

There are three known types of Alzheimer's Disease (see http://www.webmd.com/alzheimers/guide/alzheimers-types). The first type, early-onset Alzheimer's, is a rare form of Alzheimer's disease, in which people are diagnosed with the disease before age 65. Less than 10% of all Alzheimer's disease patients have this type. Younger people who develop Alzheimer's disease have more of the brain abnormalities that are associated with it. The second type, late-onset Alzheimer's, is the most common form of Alzheimer's disease, accounting for about 90% of cases, and usually occurring after age 65. Late-onset Alzheimer's disease strikes almost half of all people over the age of 85 and can or cannot be hereditary. Late-onset dementia is also called sporadic Alzheimer's disease (SOD). The last type, familial Alzheimer's disease (FAD), is a form of Alzheimer's disease that is known to be entirely inherited. In affected families, members of at least two generations have had Alzheimer's disease. FAD is extremely rare, accounting for less than 1% of all cases of Alzheimer's disease, and has a much earlier onset (often in the 40s) (see http://www.webmd.com/alzheimers/guide/alzheimers-types).

The majority of AD is sporadic (SAD), but variants in apolipoprotein E (ApoE) (Goedert M, Spillantini M G (2006) Science 314:777-781) and in SORL1, a neuronal sorting receptor (Rogaeva et al., (2007) Nature Genet. 39:168-177), can be predisposing genetic factors. At least three genes have been identified in the familial form (FAD): amyloid precursor protein (APP), presenilin-1 (PS1), and presenilin-2 (PS2).

APP is produced in large quantities in neurons and is metabolized very rapidly (Lee et al. 2008; see also O'Brien and Wong, 2011, Annu Rev Neurosci., 34: 185-204). Multiple alternate pathways exist for APP proteolysis, some of which lead to generation of the Aβ peptide and some of which do not. After sorting in the endoplasmic reticulum and Golgi, APP is delivered to the axon, where it is transported by fast axonal transport to synaptic terminals (Koo et al. 1990; see also O'Brien and Wong, 2011, Annu Rev Neurosci., 34: 185-204).

Crucial steps in APP processing occur at the cell surface and in the trans-Golgi network (TGN) (O'Brien and Wong, 2011, Annu Rev Neurosci., 34: 185-204). From the TGN, APP can be transported to the cell surface or directly to an endosomal compartment. Clathrin-associated vesicles mediate both these steps. On the cell surface, APP can be proteolyzed directly by α-secretase and then γ-secretase, a process that does not generate Aβ, or reinternalized in clathrin-coated pits into another endosomal compartment containing the proteases BACE1 and γ-secretase. The latter results in the production of Aβ, which is then dumped into the extracellular space following vesicle recycling or degraded in lysosomes. Finally, to complete the APP cycling loop, retrograde communication occurs between endosomal compartments and the TGN, mediated by a complex of molecules called retromers (O'Brien and Wong, 2011, Annu Rev Neurosci., 34: 185-204).

Rare, autosomal dominantly inherited familial forms of AD (FAD) are caused by mutations in APP or in one of two Presenilin genes that encode components of the γ-secretase enzyme complex required for APP cleavage to Aβ (Hardy and Selkoe, 2002). This has led to the amyloid hypothesis of AD pathogenesis, whereby sequential cleavage of APP by α-secretase and γ-secretase enzymes is potentiated in AD patient brain, leading to increased accumulation of the Aβ fragment, which is ultimately toxic to neurons (Hardy and Selkoe, 2002). Presenilin mutations favor the preferential accumulation of the Aβ42 isoform relative to the Aβ40 isoform. However, the amyloid hypothesis has increasingly been challenged; for instance, mutations in Presenilins can function in APP-independent pathways (De Strooper and Annaert, 2010; Parks and Curtis, 2007; Pimplikar et al., 2010; Shen and Kelleher, 2007).

It is presently unknown whether similar mechanisms govern SAD and the rare familial forms of the disease. Twin studies and population-based approaches such as genome-wide association studies point to a major genetic component to SAD risk, despite the lack of overt Mendelian inheritance (Bertram et al., 2010; Gatz et al., 2006; Mayeux and Hyslop, 2008). Common genetic variants such as the APOE4 allele increase AD risk and can act synergistically with aging and environmental factors. APOE4 is particularly relevant, as the presence of a single APOE4 allele increases SAD risk ˜4-fold (Farrer et al., 1997; Strittmatter et al., 1993); consequently, the majority of SAD patients harbor this risk allele, whereas unaffected individuals typically do not.

For AD, cell-based therapies, such as neuronal stem cells derived from the subventricular zone of animals, can provide either trophic support or replacement neurons (Blurton-Jones, M., et al. PNAS, 106, 13594-13599 (2009); Yamasaki, T. R., et al. J Neurosci 27, 11925-11933 (2007)). It is clear that structural and functional cellular plasticity is maintained in regions of the adult brain such as the dentate gyms (Deng, et al., Nat Rev Neurosci. 11, 339-350). An initial clinical trial using patient skin keratinocytes modified with a plasmid vector to express nerve growth factor (NGF) suggested this approach (Tuszynski, et al. Nat Med, 11, 551-555 (2005)). Stem cell-based disease modeling in AD can also be of high value. Patient fibroblasts have been studied extensively and can show some biochemical phenotypes associated with the disease, such as elevated processing of APP to the Aβ fragment. But a limitation of these models is that they cannot display accurately AD neuronal phenotypes, such as synaptic alterations, in the context of non-neuronal cells.

### Methods of Treatment and Prevention

The present invention relates to the discovery of an effect of a major Alzheimer's Disease risk allele, APOE4, on APP processing and APP endosomal trafficking. The invention further relates to the discovery that an antibody selective for APOE4, or an excess amount of APOE3 protein, prevents these defects. This invention provides for methods for the treatment and/or prevention of a neurodegenerative disorder by using an inhibitor of APOE4, such as an antibody inhibitor, or by using an excess of APOE3 protein.

This invention provides a human neuronal model system for the analysis of LOAD, and an APOE4 pre-LOAD state. Human skin fibroblasts from APOE4 carriers and noncarriers are converted to human induced neurons (hiNs). Defects observed in hiNs carrying APOE4 are consistent with clinical LOAD pathology. The presence of the APOE4 allele correlates with increased APP processing and increased localization to the endosomal compartment. hiNS carrying APOE4 exhibit a broad modification in vesicular endocytic trafficking from the plasma membrane to the early endosomes. A blocking antibody specific to APOE4 prevents these defects. Exogenous APOE3 protein also abolishes these defects. This invention provides a cell line that can be useful for identifying LOAD therapeutics. The use of an APOE4 antibody or exogenous APOE3 presents a new therapy for treating LOAD.

The cell system provided in the invention can be used as a human neuron model for late onset Alzheimer's disease (LOAD). Detailed studies on the mechanistic action of APOE4 in LOAD can be carried out using this model. Dissecting the APOE4 pathway using this model can identify potential drug targets for LOAD. Drug targets in the APOE4 pathway can also be used to treat additional diseases associated with the APOE4 allele, such as multiple sclerosis, traumatic brain injury, subarachnoid hemorrhage, stroke, dementia puglistica, or Parkinson's disease. This model can be used to identify compounds that prevent the LOAD pathology observed in hiN cells carrying APOE4 and that can serve as useful therapeutics for LOAD. An antibody selective for APOE4 provides a new therapy for treating LOAD. Delivery of exogenous low-risk APOE proteins to patients carrying the APOE4 allele alos presents a new therapeutic strategy for treating LOAD.

The present disclosure provides methods for the treatment and/or prevention of Alzheimer's Disease. In one aspect, the disclosure provides a method of treating, preventing or delaying the onset of sporadic Alzheimer's Disease in a subject, the method comprising administering to the subject a therapeutically effective amount of an antibody that binds to the APOE4 protein, thereby treating or preventing the Alzheimer's Disease.

In another aspect, the disclosure provides a method of treating, preventing or delaying the onset of sporadic Alzheimer's Disease in a subject, the method comprising administering to the subject a therapeutically effective amount of a peptide or a peptidomimetic that binds to the APOE4 protein, thereby treating or preventing the Alzheimer's Disease.

In another aspect, the disclosure provides a method of treating, preventing or delaying the onset of sporadic Alzheimer's Disease in a subject, the method comprising administering to the subject a therapeutically effective amount of an antisense RNA, or a siRNA, that inhibits expression of the gene that encodes the APOE4 protein, thereby treating or preventing the Alzheimer's Disease.

In one embodiment, the subject is heterozygous for the APOE4 allele. In another embodiment, the subject is homozygous for the APOE4 allele. In one embodiment, an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

In one embodiment, the antibody is a monoclonal antibody or a polyclonal antibody. In another embodiment, the APOE4 protein comprises SEQ ID NO:1.

In one embodiment, the antisense RNA or the siRNA binds to a human nucleic acid sequence comprising SEQ ID NO: 2.

In one embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the antibody. In another embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the peptide or peptidomimetic. In another embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the antisense RNA or the siRNA. In one embodiment, the levels of Aβ40, Aβ42, and sAPPβ in the subject are measured in the cerebro-spinal fluid of the subject.

In another aspect, the disclosure provides a method of treating, preventing or delaying the onset of sporadic Alzheimer's Disease in a subject, the method comprising administering to the subject a therapeutically effective amount of APOE3 protein, thereby treating or preventing the Alzheimer's Disease.

In one embodiment, the subject is heterozygous for the APOE4 allele. In another embodiment, the subject is homozygous for the APOE4 allele. In one embodiment, an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

In one embodiment, the APOE3 protein is delivered to a cell of the subject through viral-mediated delivery. In one embodiment, the APOE3 protein comprises SEQ ID NO:3.

In one embodiment, the treating or preventing comprises reducing the levels of Aβ40, Aβ42, and sAPPβ, or any combination thereof, in the subject, as compared to the levels of Aβ40, Aβ42, and sAPPβ in the subject prior to administration of the APOE3 protein. In another embodiment, the levels of Aβ40, Aβ42, and sAPPβ in the subject are measured in the cerebro-spinal fluid of the subject.

In another aspect, the disclosure provides a method of treating, preventing or delaying the onset of sporadic Alzheimer's Disease in a subject, the method comprising administering to the subject a therapeutically effective amount of an antibody that binds to the APOE2, APOE3 or APOE4 protein, or any combination thereof, thereby treating or preventing the Alzheimer's Disease.

In one embodiment, the subject is heterozygous for the APOE4 allele or the APOE2 allele. In another embodiment, the subject is homozygous for the APOE4 allele or the APOE2 allele. In one embodiment, the APOE4 protein comprises SEQ ID NO:1. In another embodiment, the APOE3 protein comprises SEQ ID NO:3.

In one embodiment, an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

In one embodiment, the antibody is a monoclonal antibody or a polyclonal antibody.

In one embodiment, the treating or preventing comprises reducing the levels of Aβ40, Aβ42, sAPPβ, or any combination thereof, in the subject, as compared to the levels of Aβ40, Aβ42, and sAPPβ in the subject prior to administration of the antibody. In another embodiment, the levels of Aβ40, Aβ42, and sAPPβ in the subject are measured in the cerebro-spinal fluid of the subject.

The present disclosure provides methods for the treatment and/or prevention of a neurodegenerative disorder. In one aspect, the present disclosure provides for a method of treating or preventing a neurodegenerative disorder in a subject, the method comprising administering to the subject a therapeutically effective amount of an antibody that binds to APOE2, APOE3 or APOE4, or any combination thereof, thereby treating or preventing the disorder. In one embodiment, the antibody is a monoclonal antibody or a polyclonal antibody.

In one embodiment, the neurodegenerative disorder is Alzheimer's Disease. In one embodiment, the Alzheimer's Disease is familial Alzheimer's Disease. In another embodiment, the Alzheimer's Disease is sporadic Alzheimer's Disease or late-onset Alzheimer's Disease.

In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease. In one embodiment, the neurodegenerative disorder is a cognitive disorder. In one embodiment, the neurodegenerative disorder is mild cognitive disorder. In another embodiment, the neurodegenerative disorder is frontotemporal dementia.

In one embodiment, the subject is heterozygous for the APOE4 allele or the APOE2 allele. In another embodiment, the subject is homozygous for the APOE4 allele or the APOE2 allele.

In one embodiment, an indicator of the neurodegenerative disorder is increased levels of Aβ40 in a cell of the subject, increased levels of Aβ42 in a cell of the subject, increased levels of APP-positive puncta in a cell of the subject, decreased localization of APP on the surface of a cell of the subject, increased levels of extracellular sAPPβ in a cell of the subject, or any combination thereof, as compared to a cell of a subject that does not have the disorder. In one embodiment, the cell is a brain cell, a neuronal cell, or a hiN cell.

In one embodiment, the APOE4 comprises SEQ ID NO:1. In another embodiment, the APOE3 comprises SEQ ID NO:3.

In one embodiment, the treating or preventing comprises reducing the levels of Aβ40, Aβ42, or both, in a cell of the subject, as compared to the levels of Aβ40, Aβ42, or both in the cell of the subject prior to administration of the antibody. In another embodiment, the treating or preventing comprises reducing the levels of APP-positive puncta in a cell of the subject, as compared to the levels of APP-positive puncta in the cell of the subject prior to administration of the antibody. In another embodiment, the treating or preventing comprises reducing the levels of extracellular APPβ in a cell of the subject, as compared to the levels of extracellular APPβ in the cell of the subject prior to administration of the antibody.

In another aspect, the present disclosure provides for a method of treating or preventing a neurodegenerative disorder in a subject, the method comprising administering to the subject an inhibitor of APOE2, APOE3 or APOE4, or any combination thereof, thereby treating or preventing the disorder.

In one embodiment, the neurodegenerative disorder is Alzheimer's Disease. In one embodiment, the Alzheimer's Disease is familial Alzheimer's Disease. In another embodiment, the Alzheimer's Disease is sporadic Alzheimer's Disease or late-onset Alzheimer's Disease.

In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease. In one embodiment, the neurodegenerative disorder is a cognitive disorder. In one embodiment, the neurodegenerative disorder is mild cognitive disorder. In another embodiment, the neurodegenerative disorder is frontotemporal dementia.

In one embodiment, the subject is heterozygous for the APOE4 allele or the APOE2 allele. In another embodiment, the subject is homozygous for the APOE4 allele or the APOE2 allele.

In one embodiment, an indicator of the neurodegenerative disorder is increased levels of Aβ40 in a cell of the subject, increased levels of Aβ42 in a cell of the subject, increased levels of APP-positive puncta in a cell of the subject, decreased localization of APP on the surface of a cell of the subject, increased levels of extracellular sAPPβ in a cell of the subject, or any combination thereof, as compared to a cell of a subject that does not have the disorder. In one embodiment, the cell is a brain cell, a neuronal cell, or a hiN cell.

In one embodiment, the APOE4 comprises SEQ ID NO:1. In another embodiment, the APOE3 comprises SEQ ID NO:3.

In one embodiment, the treating or preventing comprises reducing the levels of Aβ40, Aβ42, or both, in a cell of the subject, as compared to the levels of Aβ40, Aβ42, or both in the cell of the subject prior to administration of the inhibitor. In another embodiment, the treating or preventing comprises reducing the levels of APP-positive puncta in a cell of the subject, as compared to the levels of APP-positive puncta in the cell of the subject prior to administration of the inhibitor. In another embodiment, the treating or preventing comprises reducing the levels of extracellular APPβ in a cell of the subject, as compared to the levels of extracellular APPβ in the cell of the subject prior to administration of the inhibitor.

In one embodiment, the inhibitor comprises a small molecule. In one embodiment, the inhibitor is a peptide or a peptidomimetic that specifically binds to the APOE4 protein. In another embodiment, the inhibitor is an antibody or an antibody fragment. In one embodiment, the antibody is a monoclonal antibody or a polyclonal antibody. In another embodiment, the antibody fragment is a fragment of a monoclonal or a polyclonal antibody. In yet another embodiment, the antibody or the antibody fragment binds to a protein comprising SEQ ID NO:1. In another embodiment, the antibody or the antibody fragment binds to a protein comprising SEQ ID NO:3.

In one embodiment, the inhibitor comprises an antisense RNA that inhibits expression of the gene that encodes the APOE4 protein. In another embodiment, the inhibitor comprises a siRNA that inhibits expression of the gene that encodes the APOE4 protein. In one embodiment, the siRNA binds to a human nucleic acid sequence comprising SEQ ID NO: 2.

In one embodiment, the inhibitor comprises an antisense RNA that inhibits expression of the gene that encodes the APOE3 protein. In another embodiment, the inhibitor comprises a siRNA that inhibits expression of the gene that encodes the APOE3 protein. In one embodiment, the siRNA binds to a human nucleic acid sequence comprising SEQ ID NO: 4.

In one embodiment, the inhibitor comprises an antisense RNA that inhibits expression of the gene that encodes the APOE2 protein. In another embodiment, the inhibitor comprises a siRNA that inhibits expression of the gene that encodes the APOE2 protein.

In another aspect, the present disclosure provides a method of treating or preventing a neurodegenerative disorder in a subject, the method comprising administering to the subject a therapeutically effective amount of a APOE protein that is associated with a low risk of a neurodegenerative disease, thereby treating or preventing the disorder. In one embodiment, the APOE protein is APOE3 protein.

In one embodiment, the neurodegenerative disorder is Alzheimer's Disease. In one embodiment, the Alzheimer's Disease is familial Alzheimer's Disease. In another embodiment, the Alzheimer's Disease is sporadic Alzheimer's Disease or late-onset Alzheimer's Disease.

In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease. In one embodiment, the neurodegenerative disorder is a cognitive disorder. In one embodiment, the neurodegenerative disorder is mild cognitive disorder. In another embodiment, the neurodegenerative disorder is frontotemporal dementia.

In one embodiment, the subject is heterozygous for the APOE4 allele or the APOE2 allele. In another embodiment, the subject is homozygous for the APOE4 allele or the APOE2 allele.

In one embodiment, an indicator of the neurodegenerative disorder is increased levels of Aβ40 in a cell of the subject, increased levels of Aβ42 in a cell of the subject, increased levels of APP-positive puncta in a cell of the subject, decreased localization of APP on the surface of a cell of the subject, increased levels of extracellular sAPPβ in a cell of the subject, or any combination thereof, as compared to a cell of a subject that does not have the disorder. In one embodiment, the cell is a brain cell, a neuronal cell, or a hiN cell.

In one embodiment, the APOE3 protein comprises SEQ ID NO: 3.

In one embodiment, the treating or preventing comprises reducing the levels of Aβ40, Aβ42, or both, in a cell of the subject, as compared to the levels of Aβ40, Aβ42, or both in the cell of the subject prior to administration of the APOE protein.

In one embodiment, the treating or preventing comprises reducing the levels of APP-positive puncta in a cell of the subject, as compared to the levels of APP-positive puncta in the cell of the subject prior to administration of the APOE protein. In another embodiment, the treating or preventing comprises reducing the levels of extracellular APPβ in a cell of the subject, as compared to the levels of extracellular APPβ in the cell of the subject prior to administration of the APOE protein.

### DNA and Amino Acid Manipulation Methods and Purification Thereof

The present invention utilizes conventional molecular biology, microbiology, and recombinant DNA techniques available to one of ordinary skill in the art. Such techniques are well known to the skilled worker and are explained fully in the literature. See, e.g., Maniatis, Fritsch & Sambrook, “Molecular Cloning: A Laboratory Manual” (1982): “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986): B. Perbal, “A Practical Guide to Molecular Cloning” (1984), and Sambrook, et al., “Molecular Cloning: a Laboratory Manual” (1989).

One skilled in the art can obtain a protein in several ways, which include, but are not limited to, isolating the protein via biochemical means or expressing a nucleotide sequence encoding the protein of interest by genetic engineering methods.

A protein is encoded by a nucleic acid (including, for example, genomic DNA, complementary DNA (cDNA), synthetic DNA, as well as any form of corresponding RNA). For example, it can be encoded by a recombinant nucleic acid of a gene. The proteins of the invention can be obtained from various sources and can be produced according to various techniques known in the art. For example, a nucleic acid that encodes a protein can be obtained by screening DNA libraries, or by amplification from a natural source. A protein can be a fragment or portion thereof. The nucleic acids encoding a protein can be produced via recombinant DNA technology and such recombinant nucleic acids can be prepared by conventional techniques, including chemical synthesis, genetic engineering, enzymatic techniques, or a combination thereof.

A APOE4 protein is encoded by a nucleic acid (including, for example, genomic DNA, complementary DNA (cDNA), synthetic DNA, as well as any form of corresponding RNA). For example, it can be encoded by a recombinant nucleic acid of a APOE4 gene. The APOE4 proteins of the invention can be obtained from various sources and can be produced according to various techniques known in the art. For example, a nucleic acid that encodes a APOE4 protein can be obtained by screening DNA libraries, or by amplification from a natural source. A APOE4 protein can be a fragment or portion thereof. The nucleic acids encoding a APOE4 protein can be produced via recombinant DNA technology and such recombinant nucleic acids can be prepared by conventional techniques, including chemical synthesis, genetic engineering, enzymatic techniques, or a combination thereof. A APOE4 protein is a polypeptide encoded by the nucleic acid having the nucleotide sequence shown in SEQ ID NO: 2. An example of a APOE4 polypeptide has the amino acid sequence shown in SEQ ID NO: 1. Another example of a APOE4 polypeptide has the amino acid sequence shown in SEQ ID NO: 5.

The amino acid sequence of the processed form of human APOE4 is depicted in SEQ ID NO: 1. The amino acid sequence of the human precursor APOE4 protein is depicted in SEQ ID NO: 5. The nucleic acid sequence of human APOE4 is shown in SEQ ID NO: 2. Sequence information related to APOE4 is accessible in public databases, such as GenBank.

SEQ ID NO: 1 is the human wild type amino acid sequence corresponding to the processed form of APOE4 (residues 1-303):

GCQAKVEQAVETEPEPELRQQTEWQSGQRWELALGRFWDYLRWVQTLSEQ
VQEELLSSQVTQELRALMDETMKELKAYKSELEEQLTPVAEETRARLSKE
LQAAQARLGADMEDVRGRLVQYRGEVQAMLGQSTEELRVRLASHLRKLRK
RLLRDADDLQKRLAVYQAGAREGAERGLSAIRERLGPLVEQGRVRAATVG
SLAGQPLQERAQAWGERLRARMEEMGSRTRDRLDEVKEQVAEVRAKLEEQ
AQQIRLQAEAFQARLKSWFEPLVEDMQRQWAGLVEKVQAAVGTSAAPVPS
DNH

SEQ ID NO: 2 is the human wild type nucleic acid sequence corresponding to APOE4 (residues 1-954)

ATGAAGGTTCTGTGGGCTGCGTTGCTGGTCACATTCCTGGCAGGATGCCA
GGCCAAGGTGGAGCAAGCGGTGGAGACAGAGCCGGAGCCCGAGCTGCGCC
AGCAGACCGAGTGGCAGAGCGGCCAGCGCTGGGAACTGGCACTGGGTCGC
TTTTGGGATTACCTGCGCTGGGTGCAGACACTGTCTGAGCAGGTGCAGGA
GGAGCTGCTCAGCTCCCAGGTCACCCAGGAACTGAGGGCGCTGATGGACG
AGACCATGAAGGAGTTGAAGGCCTACAAATCGGAACTGGAGGAACAACTG
ACCCCGGTGGCGGAGGAGACGCGGGCACGGCTGTCCAAGGAGCTGCAGGC
GGCGCAGGCCCGGCTGGGCGCGGACATGGAGGACGTGCGCGGCCGCCTGG
TGCAGTACCGCGGCGAGGTGCAGGCCATGCTCGGCCAGAGCACCGAGGAG
CTGCGGGTGCGCCTCGCCTCCCACCTGCGCAAGCTGCGTAAGCGGCTCCT
CCGCGATGCCGATGACCTGCAGAAGCGCCTGGCAGTGTACCAGGCCGGGG
CCCGCGAGGGCGCCGAGCGCGGCCTCAGCGCCATCCGCGAGCGCCTGGGG
CCCCTGGTGGAACAGGGCCGCGTGCGGGCCGCCACTGTGGGCTCCCTGGC
CGGCCAGCCGCTACAGGAGCGGGCCCAGGCCTGGGGCGAGCGGCTGCGCG
CGCGGATGGAGGAGATGGGCAGCCGGACCCGCGACCGCCTGGACGAGGTG
AAGGAGCAGGTGGCGGAGGTGCGCGCCAAGCTGGAGGAGCAGGCCCAGCA
GATACGCCTGCAGGCCGAGGCCTTCCAGGCCCGCCTCAAGAGCTGGTTCG
AGCCCCTGGTGGAAGACATGCAGCGCCAGTGGGCCGGGCTGGTGGAGAAG
GTGCAGGCTGCCGTGGGCACCAGCGCCGCCCCTGTGCCCAGCGACAATCA
CTGA

A APOE3 protein is encoded by a nucleic acid (including, for example, genomic DNA, complementary DNA (cDNA), synthetic DNA, as well as any form of corresponding RNA). For example, it can be encoded by a recombinant nucleic acid of a APOE3 gene. The APOE3 proteins of the invention can be obtained from various sources and can be produced according to various techniques known in the art. For example, a nucleic acid that encodes a APOE3 protein can be obtained by screening DNA libraries, or by amplification from a natural source. A APOE3 protein can be a fragment or portion thereof. The nucleic acids encoding a APOE3 protein can be produced via recombinant DNA technology and such recombinant nucleic acids can be prepared by conventional techniques, including chemical synthesis, genetic engineering, enzymatic techniques, or a combination thereof. A APOE3 protein is a polypeptide encoded by the nucleic acid having the nucleotide sequence shown in SEQ ID NO: 4. An example of a APOE3 polypeptide has the amino acid sequence shown in SEQ ID NO: 3. Another example of a APOE3 polypeptide has the amino acid sequence shown in SEQ ID NO: 6.

The amino acid sequence of the processed form of human APOE3 is depicted in SEQ ID NO: 3. The amino acid sequence of the human precursor APOE3 protein is depicted in SEQ ID NO: 6. The nucleic acid sequence of human APOE3 is shown in SEQ ID NO: 4. Sequence information related to APOE3 is accessible in public databases, such as GenBank.

SEQ ID NO: 3 is the human wild type amino acid sequence corresponding to the processed form of APOE3 (residues 1-303):

GCQAKVEQAVETEPEPELRQQTEWQSGQRWELALGRFWDYLRWVQTLSEQ
VQEELLSSQVTQELRALMDETMKELKAYKSELEEQLTPVAEETRARLSKE
LQAAQARLGADMEDVCGRLVQYRGEVQAMLGQSTEELRVRLASHLRKLRK
RLLRDADDLQKRLAVYQAGAREGAERGLSAIRERLGPLVEQGRVRAATVG
SLAGQPLQERAQAWGERLRARMEEMGSRTRDRLDEVKEQVAEVRAKLEEQ
AQQIRLQAEAFQARLKSWFEPLVEDMQRQWAGLVEKVQAAVGTSAAPVPS
DNH

SEQ ID NO: 4 is the human wild type nucleic acid sequence corresponding to APOE3 (residues 1-954)

ATGAAGGTTCTGTGGGCTGCGTTGCTGGTCACATTCCTGGCAGGATGCCA
GGCCAAGGTGGAGCAAGCGGTGGAGACAGAGCCGGAGCCCGAGCTGCGCC
AGCAGACCGAGTGGCAGAGCGGCCAGCGCTGGGAACTGGCACTGGGTCGC
TTTTGGGATTACCTGCGCTGGGTGCAGACACTGTCTGAGCAGGTGCAGGA
GGAGCTGCTCAGCTCCCAGGTCACCCAGGAACTGAGGGCGCTGATGGACG
AGACCATGAAGGAGTTGAAGGCCTACAAATCGGAACTGGAGGAACAACTG
ACCCCGGTGGCGGAGGAGACGCGGGCACGGCTGTCCAAGGAGCTGCAGGC
GGCGCAGGCCCGGCTGGGCGCGGACATGGAGGACGTGTGCGGCCGCCTGG
TGCAGTACCGCGGCGAGGTGCAGGCCATGCTCGGCCAGAGCACCGAGGAG
CTGCGGGTGCGCCTCGCCTCCCACCTGCGCAAGCTGCGTAAGCGGCTCCT
CCGCGATGCCGATGACCTGCAGAAGCGCCTGGCAGTGTACCAGGCCGGGG
CCCGCGAGGGCGCCGAGCGCGGCCTCAGCGCCATCCGCGAGCGCCTGGGG
CCCCTGGTGGAACAGGGCCGCGTGCGGGCCGCCACTGTGGGCTCCCTGGC
CGGCCAGCCGCTACAGGAGCGGGCCCAGGCCTGGGGCGAGCGGCTGCGCG
CGCGGATGGAGGAGATGGGCAGCCGGACCCGCGACCGCCTGGACGAGGTG
AAGGAGCAGGTGGCGGAGGTGCGCGCCAAGCTGGAGGAGCAGGCCCAGCA
GATACGCCTGCAGGCCGAGGCCTTCCAGGCCCGCCTCAAGAGCTGGTTCG
AGCCCCTGGTGGAAGACATGCAGCGCCAGTGGGCCGGGCTGGTGGAGAAG
GTGCAGGCTGCCGTGGGCACCAGCGCCGCCCCTGTGCCCAGCGACAATCA
CTGA.

SEQ ID NO: 5 is the human wild type amino acid sequence corresponding to the precursor APOE4 protein (residues 1-317):

MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVRGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH

SEQ ID NO: 6 is the human wild type amino acid sequence corresponding to the precursor APOE3 protein (residues 1-317):

MKVLWAALLVTFLAGCQAKVEQAVETEPEPELRQQTEWQSGQRWELALGR
FWDYLRWVQTLSEQVQEELLSSQVTQELRALMDETMKELKAYKSELEEQL
TPVAEETRARLSKELQAAQARLGADMEDVCGRLVQYRGEVQAMLGQSTEE
LRVRLASHLRKLRKRLLRDADDLQKRLAVYQAGAREGAERGLSAIRERLG
PLVEQGRVRAATVGSLAGQPLQERAQAWGERLRARMEEMGSRTRDRLDEV
KEQVAEVRAKLEEQAQQIRLQAEAFQARLKSWFEPLVEDMQRQWAGLVEK
VQAAVGTSAAPVPSDNH

Sequence information related to APOE2, including nucleic acid and amino acid sequence information, is accessible in public databases, such as GenBank.

Protein Variants:

Protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Techniques for making substitution mutations at predetermined sites in DNA having a known sequence are well known, for example M13 primer mutagenesis and PCR mutagenesis. Amino acid substitutions can be single residues, but can occur at a number of different locations at once. In one non-limiting embodiment, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and should not create complementary regions that can produce secondary mRNA structure. Substitutional variants are those in which at least one residue has been removed and a different residue inserted in its place.

Substantial changes in function or immunological identity are made by selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that can produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of proteins are provided by the present invention. The variations in the amino acid sequence can be when the sequence maintains at least about 30%, at least about 40%, at least about 50%, at least about 60%, at least about 70%, at least about 75%, at least about 80%, at least about 90%, at least about 95%, or at least about 99% identity to SEQ ID NOs: 1, 3, 5 and 6. For example, conservative amino acid replacements can be utilized. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Bacterial and Yeast Expression Systems.

In bacterial systems, a number of expression vectors can be selected. For example, when a large quantity of a protein encoded by a gene, such as APOE4 or APOE3, or APOE2 is needed for the induction of antibodies, vectors which direct high level expression of proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems.

If plant expression vectors are used, the expression of sequences encoding a APOE4 or APOE3 or APOE2 protein can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express APOE4 or APOE3 or APOE2 proteins. For example, in one such system Autographa californica nuclear polyhedrosis virus (AcNPV) is used as a vector to express foreign genes in Spodoptera frugiperda cells or in Trichoplusia larvae. Sequences encoding a polypeptide of APOE4 or APOE3 or APOE2 can be cloned into a non-essential region of the virus, such as the polyhedrin gene, and placed under control of the polyhedrin promoter. Successful insertion of nucleic acid sequences, such as a sequence corresponding to a gene, such as a APOE4 or APOE3 or APOE2 gene, will render the polyhedrin gene inactive and produce recombinant virus lacking coat protein. The recombinant viruses can then be used to infect S. frugiperda cells or Trichoplusia larvae in which the protein or a variant thereof can be expressed.

Mammalian Expression Systems.

An expression vector can include a nucleotide sequence that encodes a APOE4 or APOE3 or APOE2 polypeptide linked to at least one regulatory sequence in a manner allowing expression of the nucleotide sequence in a host cell. A number of viral-based expression systems can be used to express a APOE4 or APOE3 or APOE2 protein or a variant thereof in mammalian host cells. For example, if an adenovirus is used as an expression vector, sequences encoding a protein can be ligated into an adenovirus transcription/translation complex comprising the late promoter and tripartite leader sequence. Insertion into a non-essential E1 or E3 region of the viral genome can be used to obtain a viable virus which expresses a APOE4 or APOE3 or APOE2 protein in infected host cells. Transcription enhancers, such as the Rous sarcoma virus (RSV) enhancer, can also be used to increase expression in mammalian host cells.

Regulatory sequences are well known in the art, and can be selected to direct the expression of a protein or polypeptide of interest in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L, et al., Curr Opin Genet Dev, 2002, 12(2):137-41) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, a protein encoded by a gene, such as APOE4 or APOE3 or APOE2).

Cell Transfection

A eukaryotic expression vector can be used to transfect cells in order to produce proteins encoded by nucleotide sequences of the vector. Mammalian cells, such as neuronal cells or brain tissue, can contain an expression vector (for example, one that contains a gene encoding a APOE4 or APOE3 or APOE2 protein or polypeptide) via introducing the expression vector into an appropriate host cell via methods known in the art.

A host cell strain can be chosen for its ability to modulate the expression of the inserted sequences or to process the expressed polypeptide encoded by a gene, such as a APOE4 or APOE3 or APOE2 gene, in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextran-mediated transfection, or electroporation. Electroporation is carried out at approximate voltage and capacitance to result in entry of the DNA construct(s) into cells of interest (such as neuronal cells and brain cells). Other transfection methods also include modified calcium phosphate precipitation, polybrene precipitation, liposome fusion, and receptor-mediated gene delivery.

Cells that will be genetically engineered can be primary and secondary cells obtained from various tissues, and include cell types which can be maintained and propagated in culture. Non-limiting examples of primary and secondary cells include epithelial cells, neuronal cells, brain cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells. In another embodiment, removal of a hair follicle can be used to obtain a source of fibroblasts, keratinocytes, endothelial cells, or mesenchymal cells. A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in United States Patent Application Publication 2004/0057937 and PCT application publication WO 2001/32840, and are hereby incorporated by reference.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, a APOE4 or APOE3 protein or polypeptide).

Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992), which is incorporated herein by reference in its entirety). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the present invention is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of neuronal cells, brain cells, fibroblast cells, or hiN cells. In a further embodiment, neuronal cells, brain cells, fibroblast cells, or hiN cells, or any other mammalian cells, can be transfected with DNA vectors containing genes that encode a polypeptide or protein of interest (for example, a APOE4 or APOE3 or APOE2 protein or polypeptide). In other embodiments of the invention, cells are grown in a suspension culture (for example, a three-dimensional culture such as a hanging drop culture) in the presence of an effective amount of enzyme, wherein the enzyme substrate is an extracellular matrix molecule in the suspension culture. For example, the enzyme can be a hyaluronidase.

A suspension culture is a type of culture wherein cells, or aggregates of cells (such as aggregates of DP cells), multiply while suspended in liquid medium. A suspension culture comprising mammalian cells can be used for the maintenance of cell types that do not adhere or to enable cells to manifest specific cellular characteristics that are not seen in the adherent form. Some types of suspension cultures can include three-dimensional cultures or a hanging drop culture. A hanging-drop culture is a culture in which the material to be cultivated is inoculated into a drop of fluid attached to a flat surface (such as a coverglass, glass slide, Petri dish, flask, and the like), and can be inverted over a hollow surface. Cells in a hanging drop can aggregate toward the hanging center of a drop as a result of gravity. However, according to the methods of the invention, cells cultured in the presence of a protein that degrades the extracellular matrix (such as collagenase, chondroitinase, hyaluronidase, and the like) will become more compact and aggregated within the hanging drop culture, for degradation of the ECM will allow cells to become closer in proximity to one another since less of the ECM will be present. See also International PCT Publication No. WO2007/100870, which is incorporated by reference.

Cells can be cultured as a single, homogenous population in a hanging drop culture, so as to generate an aggregate of cells, or can be cultured as a heterogeneous population in a hanging drop culture so as to generate a chimeric aggregate of cells.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73).

Cells suitable for culturing according to methods of the invention can harbor introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 2001, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

Obtaining and Purifying Polypeptides

A polypeptide molecule encoded by a gene, such as a APOE4 or APOE3 or APOE2 gene, or a variant thereof, can be obtained by purification from human cells expressing a protein or polypeptide encoded by a APOE4 or APOE3 or APOE2 gene via in vitro or in vivo expression of a nucleic acid sequence encoding a APOE4 or APOE3 or APOE2 protein or polypeptide; or by direct chemical synthesis.

Detecting Polypeptide Expression.

Host cells which contain a nucleic acid encoding a APOE4 or APOE3 or APOE2 protein or polypeptide, and which subsequently express a protein encoded by a APOE4 or APOE3 or APOE2 gene, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a APOE4 or APOE3 or APOE2 protein or polypeptide can be detected by DNA-DNA or DNA-RNA hybridization or amplification using probes or fragments of nucleic acids encoding a APOE4 or APOE3 or APOE2 protein or polypeptide. In one embodiment, a fragment of a nucleic acid of a APOE4 or APOE3 gene can encompass any portion of at least about 8 consecutive nucleotides of SEQ ID NO: 2 or 4. In another embodiment, the fragment can comprise at least about 10 consecutive nucleotides, at least about 15 consecutive nucleotides, at least about 20 consecutive nucleotides, or at least about 30 consecutive nucleotides of SEQ ID NO: 2 or 4. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding a polypeptide encoded by a APOE4 or APOE3 or APOE2 gene to detect transformants which contain a nucleic acid encoding a APOE4 or APOE3 or APOE2 protein or polypeptide.

Protocols for detecting and measuring the expression of a polypeptide encoded by a gene, such as a APOE4 or APOE3 or APOE2 gene, using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a polypeptide encoded by a gene, such as a APOE4 or APOE3 or APOE2 gene, can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding a protein, such as APOE4 or APOE3 or APOE2, include, but are not limited to, oligolabeling, nick translation, end-labeling, or PCR amplification using a labeled nucleotide.

Alternatively, nucleic acid sequences encoding a polypeptide encoded by a gene, such as a APOE4 or APOE3 or APOE2 gene, can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Expression and Purification of Polypeptides.

Host cells transformed with a nucleic acid sequence encoding a polypeptide, such as APOE4 or APOE3 or APOE2, can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a polypeptide, such as APOE4 or APOE3 or APOE2, can be designed to contain signal sequences which direct secretion of soluble polypeptide molecules encoded by a gene, such as a APOE4 or APOE3 or APOE2 gene, or a variant thereof, through a prokaryotic or eukaryotic cell membrane or which direct the membrane insertion of membrane-bound a polypeptide molecule encoded by a APOE4 or APOE3 or APOE2 gene or a variant thereof.

Other constructions can also be used to join a gene sequence encoding a APOE4 or APOE3 or APOE2 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a polypeptide encoded by a APOE4 or APOE3 or APOE2 gene also can be used to facilitate purification. One such expression vector provides for expression of a fusion protein containing a polypeptide encoded by a APOE4 or APOE3 or APOE2 gene and 6 histidine residues preceding a thioredoxin or an enterokinase cleavage site. The histidine residues facilitate purification by immobilized metal ion affinity chromatography, while the enterokinase cleavage site provides a means for purifying the polypeptide encoded by a APOE4 or APOE3 or APOE2 gene.

A APOE4 or APOE3 or APOE2 polypeptide can be purified from any human or non-human cell which expresses the polypeptide, including those which have been transfected with expression constructs that express a APOE4 or APOE3 or APOE2 protein. A purified APOE4 or APOE3 or APOE2 protein can be separated from other compounds which normally associate with a protein encoded by a APOE4 or APOE3 or APOE2 gene in the cell, such as certain proteins, carbohydrates, or lipids, using methods practiced in the art. Non-limiting methods include size exclusion chromatography, ammonium sulfate fractionation, ion exchange chromatography, affinity chromatography, and preparative gel electrophoresis.

Chemical Synthesis.

Nucleic acid sequences comprising a gene, such as a APOE4 or APOE3 or APOE2 gene, that encodes a polypeptide can be synthesized, in whole or in part, using chemical methods known in the art. Alternatively, a polypeptide, such as APOE4 or APOE3 or APOE2, can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of APOE4 or APOE3 or APOE2 polypeptides can be separately synthesized and combined using chemical methods to produce a full-length molecule. In one embodiment, a fragment of a nucleic acid sequence that comprises a APOE4 or APOE3 gene can encompass any portion of at least about 8 consecutive nucleotides of SEQ ID NO: 2 or 4. In one embodiment, the fragment can comprise at least about 10 nucleotides, at least about 15 nucleotides, at least about 20 nucleotides, or at least about 30 nucleotides of SEQ ID NO: 2 or 4. Fragments include all possible nucleotide lengths between about 8 and about 100 nucleotides, for example, lengths between about 15 and about 100 nucleotides, or between about 20 and about 100 nucleotides.

A APOE4 or APOE3 or APOE2 fragment can be a fragment of a protein, such as APOE4 or APOE3 or APOE2. For example, the APOE4 or APOE3 fragment can encompass any portion of at least about 8 consecutive amino acids of SEQ ID NO: 1, 3, 5 or 6. The fragment can comprise at least about 10 consecutive amino acids, at least about 20 consecutive amino acids, at least about 30 consecutive amino acids, at least about 40 consecutive amino acids, a least about 50 consecutive amino acids, at least about 60 consecutive amino acids, at least about 70 consecutive amino acids, or at least about 75 consecutive amino acids of SEQ ID NO: 1, 3, 5 or 6. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

A synthetic peptide can be substantially purified via high performance liquid chromatography (HPLC). The composition of a synthetic polypeptide of APOE4 or APOE3 or APOE2 can be confirmed by amino acid analysis or sequencing. Additionally, any portion of an amino acid sequence comprising a protein encoded by a APOE4 or APOE3 or APOE2 gene can be altered during direct synthesis and/or combined using chemical methods with sequences from other proteins to produce a variant polypeptide or a fusion protein.

### Methods for Screening

The disclosure also provides methods for the identification of a compound or a combination of compounds that is/are useful for the prevention or treatment of a neurodegenerative disorder.

In one embodiment, the neurodegenerative disorder is Alzheimer's Disease. In one embodiment, the Alzheimer's Disease is familial Alzheimer's Disease. In another embodiment, the Alzheimer's Disease is sporadic Alzheimer's Disease or late-onset Alzheimer's Disease.

In one embodiment, the neurodegenerative disorder is dementia. In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease.

As used herein, a compound can be, but is not limited to, a compound that interacts with a APOE4 or APOE3 or APOE2 gene, or an a APOE4 or APOE3 or APOE2 protein, polypeptide, or peptide, and modulates its activity or its expression. Some non-limiting examples of compounds include peptides (such as peptide fragments comprising a polypeptide encoded by a APOE4 or APOE3 or APOE2 gene, or antibodies or fragments thereof), small molecules, and nucleic acids (such as siRNA or antisense RNA specific for a nucleic acid comprising a APOE4 or APOE3 or APOE2 gene). The compound can either increase the activity or expression of a protein encoded by a APOE4 or APOE3 or APOE2 gene, or the compound can decrease the activity or expression of a protein encoded by a APOE4 or APOE3 or APOE2 gene.

The compound can be a APOE4 antagonist or APOE3 antagonist or APOE2 antagonist (e.g., a APOE4 inhibitor, or a APOE3 inhibitor, or a APOE2 inhibitor). Antagonists of a APOE4 or APOE3 or APOE2 protein can be molecules which, when bound to a APOE4 or APOE3 or APOE2 protein, respectively, decrease the amount or the duration of the activity of the APOE4 or APOE3 or APOE2 protein, respectively. Antagonists and inhibitors include proteins, nucleic acids, antibodies, small molecules, or any other molecules which decrease the activity of a APOE4 or APOE3 or APOE2 protein.

The compound can be a APOE4 agonist or APOE3 agonist or or APOE2 agonist. Agonists of a APOE4 or APOE3 or APOE2 protein can be molecules which, when bound to a APOE4 or APOE3 or APOE2 protein, increase or prolong the activity of a APOE4 or APOE3 or APOE2 protein, respectively. APOE4 or APOE3 or APOE2 agonists include, but are not limited to, proteins, nucleic acids, small molecules, or any other molecules which activate a APOE4 or APOE3 or APOE2 protein.

In one aspect, the method comprises administering the compound or combination of compounds to an animal that is a model of a neurodegenerative disorder, such as Alzheimer's Disease, and determining whether the compound or combination of compounds improves the symptoms of a neurodegenerative disorder, such as Alzheimer's Disease, in the animal, compared to an animal not so treated.

The methods can comprise the identification of test compounds or agents (e.g., peptides (such as antibodies or fragments thereof), small molecules, nucleic acids (such as siRNA or antisense RNA), or other agents) that can treat or prevent a neurodegenerative disorder in a subject.

In one embodiment, a compound can be a peptide fragment. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The peptide fragments can be isolated from a natural source, genetically engineered, or chemically prepared. These methods are well known in the art.

A compound, for example, an agonist or antagonist of APOE4 or APOE3 or APOE2, can be a protein, such as an antibody (monoclonal, polyclonal, humanized, chimeric, or fully human), or a binding fragment thereof. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

A compound, for example, an agonist or antagonist of APOE4 or APOE3 or APOE2, can be selected from the group comprising: siRNA; interfering RNA or RNAi; dsRNA; RNA Polymerase III transcribed DNAs; ribozymes; and antisense nucleic acids, which can be RNA, DNA, or an artificial nucleic acid. Antisense oligonucleotides, including antisense DNA, RNA, and DNA/RNA molecules, act to directly block the translation of mRNA by binding to targeted mRNA, such as mRNA of APOE4 or APOE3 or APOE2, and preventing protein translation. Antisense oligonucleotides of at least about 15 bases can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McManus and Sharp (2002) Nat Rev Genetics, 3:737-47, and Sen and Blau (2006) FASEB J., 20:1293-99, the entire disclosures of which are herein incorporated by reference.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. No. 7,294,504 and U.S. Pat. No. 7,422,896, the entire disclosures of which are herein incorporated by reference). Exemplary methods for producing and testing dsRNA or siRNA molecules are described in U.S. Patent Application Publication No. 2002/0173478 to Gewirtz, U.S. Patent Application Publication No. 2007/0072204 to Hannon et al., and in U.S. Patent Application Publication No. 2004/0018176 to Reich et al., the entire disclosures of which are herein incorporated by reference. In one embodiment, an siRNA directed to a human nucleic acid sequence comprising a APOE4 or APOE3 or APOE2 gene can be generated against any one of SEQ ID NOS: 2, or 4. RNA polymerase III transcribed DNAs contain promoters, such as the U6 promoter. These DNAs can be transcribed to produce small hairpin RNAs in the cell that can function as siRNA or linear RNAs that can function as antisense RNA. A compound, for example, an agonist or antagonist of APOE4 or APOE3 or APOE2, can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

A compound, for example, an agonist or antagonist of APOE4 or APOE3 or APOE2, can be a small molecule that binds to a protein and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

Knowledge of the primary sequence of a molecule of interest, such as the amino acid sequence of APOE4 or APOE3 or APOE2, and the similarity of that sequence with proteins of known function, can provide information as to the inhibitors or antagonists of the protein of interest in addition to agonists. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, for example, an agonist or antagonist of APOE4 or APOE3 or APOE2, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), AMRI (Albany, N.Y.), ChemBridge (San Diego, Calif.), and MicroSource (Gaylordsville, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. For example, libraries can also include, but are not limited to, peptide-on-plasmid libraries, synthetic small molecule libraries, aptamer libraries, in vitro translation-based libraries, polysome libraries, synthetic peptide libraries, neurotransmitter libraries, and chemical libraries.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383. Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318. In vitro translation-based libraries include but are not limited to those described in PCT Publication No. WO 91/05058; and Mattheakis et al., (1994) Proc. Natl. Acad. Sci. USA 91:9022-9026.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Computer modeling and searching technologies permit the identification of compounds, or the improvement of already identified compounds, that can treat or prevent a neurodegenerative disorder. Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing such structures involves the steps of: (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

Screening Assays.

Test compounds or agents can be identified by two types of assays: (a) cell-based assays; or (b) cell-free assays. The assay can be a binding assay comprising direct or indirect measurement of the binding of a test compound. The assay can also be an activity assay comprising direct or indirect measurement of the activity of a compound. The assay can also be an expression assay comprising direct or indirect measurement of the expression of mRNA nucleic acid sequences or a protein encoded by a gene of interest. The various screening assays can be combined with an in vivo assay comprising measuring the effect of the test compound on the symptoms of a neurodegenerative disorder, or on the defects observed in hiN cells carrying the APOE4 allele, such as, but not limited to, increased APP processing, increased levels of Aβ40 in a cell of the subject, increased levels of Aβ42 in a cell of the subject, increased levels of APP-positive puncta in a cell of the subject, decreased localization of APP on the surface of a cell of the subject, increased levels of extracellular sAPPβ in a cell of the subject, and increased localization to the endosomal compartment, or any combination thereof. An in vivo assay can also comprise assessing the effect of a test compound on a neurodegenerative disorder in known mammalian models.

Assays for screening test compounds that bind to or modulate the activity of a protein of interest, such as, but not limited to, APOE4 or APOE3 or APOE2, can also be carried out. The test compound can be obtained by any suitable means, such as from conventional compound libraries. Determining the ability of the test compound to bind to a membrane-bound form of the protein can be accomplished via coupling the test compound with a radioisotope or enzymatic label such that binding of the test compound to the cell expressing a protein of interest can be measured by detecting the labeled compound in a complex. For example, the test compound can be labeled with ³H, ¹⁴C, ³⁵S, or ¹²⁵I, either directly or indirectly, and the radioisotope can be subsequently detected by direct counting of radioemmission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

A protein of interest, such as, but not limited to, APOE4 or APOE3 or APOE2, or the target of a protein of interest can be immobilized to facilitate the separation of complexed from uncomplexed forms of one or both of the proteins. Binding of a test compound to a protein of interest or a variant thereof, or interaction of a protein of interest with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix (for example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtiter plates).

A protein of interest, such as, but not limited to, APOE4 or APOE3 or APOE2, or a variant thereof, can also be immobilized via being bound to a solid support. Non-limiting examples of suitable solid supports include glass or plastic slides, tissue culture plates, microtiter wells, tubes, silicon chips, or particles such as beads (including, but not limited to, latex, polystyrene, or glass beads). Any method known in the art can be used to attach a polypeptide (or polynucleotide) or a variant thereof, or test compound to a solid support, including use of covalent and non-covalent linkages, or passive absorption.

The screening methods of the invention can also involve monitoring the expression of a protein of interest, such as, but not limited to, APOE4 or APOE3 or APOE2. For example, regulators of the expression of a protein of interest can be identified via contacting a cell with a test compound and determining the expression of a protein of interest in the cell. The expression level of a protein of interest in the cell in the presence of the test compound is compared to the expression level of a protein of interest in the absence of the test compound. The test compound can then be identified as a regulator of the expression of a protein of interest based on this comparison. For example, when expression of a protein of interest in the cell is statistically or significantly greater in the presence of the test compound than in its absence, the test compound is identified as a stimulator/enhancer of expression of a protein of interest in the cell. Alternatively, when expression of a protein of interest in the cell is statistically or significantly less in the presence of the test compound than in its absence, the compound is identified as an inhibitor of the expression of a protein of interest in the cell. The test compound can also be said to be an antagonist. The methods to determine the expression level of a protein encoded by a gene or mRNA of interest in the cell are well known in the art.

For binding assays, the test compound can be a small molecule which binds to and occupies the binding site of a polypeptide encoded by a gene of interest, such as APOE4 or APOE3 or APOE2, or a variant thereof. This can make the ligand binding site inaccessible to substrates, such that normal biological activity is prevented. Examples of such small molecules include, but are not limited to, small peptides or peptide-like molecules. In binding assays, either the test compound or a polypeptide encoded by a gene of interest can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide encoded by a gene of interest can then be determined via direct counting of radioemmission, by scintillation counting, or by determining conversion of an appropriate substrate to a detectable product.

Determining the ability of a test compound to bind to a protein of interest also can be accomplished using real-time Biamolecular Interaction Analysis (BIA) [McConnell et al., 1992, Science 257, 1906-1912; Sjolander, Urbaniczky, 1991, Anal. Chem. 63, 2338-2345]. BIA is a technology for studying biospecific interactions in real time, without labeling any of the interactants (for example, BIA-Core™). Changes in the optical phenomenon surface plasmon resonance (SPR) can be used as an indication of real-time reactions between biological molecules.

To identify other proteins which bind to or interact with a protein of interest and modulate its activity, a polypeptide encoded by a gene of interest can be used as a bait protein in a two-hybrid assay or three-hybrid assay (Szabo et al., 1995, Curr. Opin. Struct. Biol. 5, 699-705; U.S. Pat. No. 5,283,317), according to methods practiced in the art. The two-hybrid system is based on the modular nature of most transcription factors, which consist of separable DNA-binding and activation domains.

Functional Assays.

Compounds can be tested for the ability to increase or decrease the activity of a protein of interest, such as, but not limited to, APOE4 or APOE3 or APOE2, or a variant thereof. Activity can be measured after contacting a purified protein of interest, a cell membrane preparation, or an intact cell with a test compound. A test compound that decreases the activity of a protein of interest by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for decreasing the activity of a protein of interest, for example an antagonist. A test compound that increases the activity of a protein of interest by at least about 10%, at least about 20%, at least about 30%, at least about 40%, at least about 50%, at least about 60%, at least about 70%, at least about 75%, at least about 80%, at least about 90%, at least about 95% or 100% is identified as a potential agent for increasing the activity of a protein of interest, for example an agonist.

### Compounds

The present disclosure provides methods for the treatment and/or prevention of a neurodegenerative disorder, the method comprising administration of one or more compounds to a subject in need thereof, thereby treating or preventing the disorder.

In one embodiment, the neurodegenerative disorder is Alzheimer's Disease. In another embodiment, the neurodegenerative disorder is Familial Alzheimer's Disease (FAD). In another embodiment, the neurodegenerative disorder is Sporadic Alzheimer's Disease. In one embodiment, the neurodegenerative disorder is Early-Onset Alzheimer's Disease. In another embodiment, the neurodegenerative disorder is Late-Onset Alzheimer's Disease (LOAD).

In one embodiment, the neurodegenerative disorder is dementia. In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease.

As used herein, a compound can be, but is not limited to, a compound that interacts with a APOE4 or APOE3 or APOE2 gene, or an a APOE4 or APOE3 or APOE2 protein or polypeptide, or peptide, and modulates its activity or its expression. The compound can either increase the activity or expression of a protein encoded by a APOE4 or APOE3 or APOE2 gene, or the compound can decrease the activity or expression of a protein encoded by a APOE4 or APOE3 or APOE2 gene. Some non-limiting examples of compounds include peptides (such as peptide fragments comprising a polypeptide encoded by a APOE4 or APOE3 or APOE2 gene, or antibodies or fragments thereof), small molecules, and nucleic acids (such as siRNA or antisense RNA specific for a nucleic acid comprising a APOE4 or APOE3 or APOE2 gene). The compound can be a APOE4 or APOE3 or APOE2 antagonist (e.g., a APOE4 inhibitor, or a APOE3 inhibitor, or an APOE2 inhibitor). The compound can be a APOE4 or APOE3 or APOE2 agonist (e.g., an activator of APOE4 or APOE3 or APOE2). Agonists of a APOE4 or APOE3 or APOE2 protein can be molecules which, when bound to a APOE4 or APOE3 or APOE2 protein, increase or prolong the activity of the APOE4 or APOE3 or APOE2 protein. APOE4 or APOE3 or APOE2 agonists and activators include, but are not limited to, proteins, nucleic acids, small molecules, or any other molecules which activate a APOE4 or APOE3 or APOE2 protein. Antagonists of a APOE4 or APOE3 or APOE2 protein can be molecules which, when bound to a APOE4 or APOE3 or APOE2 protein, decrease the amount or the duration of the activity of the APOE4 or APOE3 or APOE2 protein. APOE4 or APOE3 or APOE2 antagonists and inhibitors include, but are not limited to, proteins, nucleic acids, antibodies, small molecules, or any other molecule which decrease the activity of a APOE4 or APOE3 or APOE2 protein.

As used herein, a “APOE4 inhibitor” or “inhibitor of APOE4” refers to a compound that interacts with a APOE4 gene or a APOE4 protein or polypeptide, and inhibits its activity and/or its expression. The compound can decrease the activity or expression of a protein encoded by APOE4.

As used herein, a “APOE3 inhibitor” or “inhibitor of APOE3” refers to a compound that interacts with a APOE3 gene or a APOE3 protein or polypeptide, and inhibits its activity and/or its expression. The compound can decrease the activity or expression of a protein encoded by APOE3.

As used herein, a “APOE2 inhibitor” or “inhibitor of APOE2” refers to a compound that interacts with a APOE2 gene or a APOE2 protein or polypeptide, and inhibits its activity and/or its expression. The compound can decrease the activity or expression of a protein encoded by APOE2.

As used herein, a “APOE4 activator” or “activator of APOE4” refers to a compound that interacts with a APOE4 gene or a APOE4 protein or polypeptide, and enhances its activity and/or its expression. The compound can increase the activity or expression of a protein encoded by APOE4.

As used herein, a “APOE3 activator” or “activator of APOE3” refers to a compound that interacts with a APOE3 gene or a APOE3 protein or polypeptide, and enhances its activity and/or its expression. The compound can increase the activity or expression of a protein encoded by APOE3.

As used herein, a “APOE2 activator” or “activator of APOE2” refers to a compound that interacts with a APOE2 gene or a APOE2 protein or polypeptide, and enhances its activity and/or its expression. The compound can increase the activity or expression of a protein encoded by APOE2.

Any suitable agonist or antagonist, inhibitor or activator, of the APOE4, APOE3, or APOE2 gene or protein, can be used. Such compounds may be, for example, small molecule drugs, peptide agents, peptidomimetic agents, antibodies (including, but not limited to monoclonal, poycloncal, humanized, and fully human antibodies, as well as antibody fragments), inhibitory RNA molecules (such as siRNA) and the like. One of skill in the art will understand that these and other types of agents may be used to inhibit or activate APOE4, or inhibit or activate APOE3, or inhibit or activate APOE2.

Nucleotide-Based Compounds

In one aspect, a compound of the invention is a nucleotide-based agonist or antagonist, inhibitor or activator, of APOE4 or APOE3 or APOE2. Such inhibitors or antagonists include, but are not limited to siRNAs, shRNAs, dsRNAs, microRNAs, antisense RNA molecules, and ribozymes, that inhibit the expression or activity of APOE4 or APOE3 or APOE2. Such nucleotide-based inhibitors may comprise ribonucleotides, deoxyribonucleotides, or various artificial nucleotide derivatives.

siRNA

RNA interference (RNAi) is a method of gene-specific silencing which employs sequence-specific small interfering RNA (siRNA) to target and degrade the gene-specific mRNA prior to translation. Methods for designing specific siRNAs based on an mRNA sequence are well known in the art and design algorithms are available on the websites of many commercial vendors that synthesize siRNAs, including Dharmacon, Ambion, Qiagen, GenScript and Clontech.

The invention provides for a nucleic acid comprising a siRNA nucleotide sequence that binds to a human nucleic acid sequence comprising SEQ ID NO: 2 or SEQ ID NO:4. The invention provides for a nucleic acid comprising a siRNA nucleotide sequence that binds to the nucleic acid sequence of APOE3, APOE4, or APOE2.

### Antisense

Antisense oligonucleotides (ASOs) are small deoxy-oligonucleotides with a sequence complementary to the mRNA of the target gene (Crooke, (1993) Curr. Opin. Invest. Drugs, 2: 1045-1048; Stein and Cheng, (1993) Science, 261: 1004-10012; Hawley and Gibson (1996) Antisense & Nucleic Drug Dev., 6: 185-195; Crooke, S. T. (2003) Ann. Rev. Med., 55: 61-95; Kalota, et al., (2004) Cancer Biol. & Therapy, 3: 4-12; Orr, et al., (2005) Meth. Mol. Med., 106: 85-111). They bind to the target mRNA through complementary base-pairing and attract the binding of RNase H, an enzyme that degrades double strand RNA, thus destroying the target mRNA (18-25). While unmodified ASOs can be as sensitive to degradation as RNA, chemical modification of the phosphodiester backbones can make them resistant to the degradative action of nucleases in in vivo situations (nonlimiting examples include phosphorothioate- or 2′-O-[2-methoxyethyl]-backbone modifications) (Monia, et al. (1996) J. Biol. Chem., 271: 14533-1440; also see U.S. Pat. Nos. 5,652,355 and 5,652,356).

ASOs offer many unique aspects that make them likely to be rapidly translated into clinical trials in humans with a neurodegenerative disorder, such as Alzheimer's Disease: 1) they are simple defined chemical agents can be synthesized in bulk under highly controlled (good clinical practice) conditions; 2) they can be delivered to patients systemically in controlled doses, making it more likely that they can even reach distal metastases; 3) they are not known to have potential for genetic damage, as with other biological agents (viruses) that are being developed and tested for gene therapy strategies and; 4) gene-targeting ASO agents are already in clinical trials for several different cancers, thus there already is a body of literature regarding their use in humans.

The invention provides for a nucleic acid comprising an ASO that binds to a human nucleic acid sequence comprising SEQ ID NO: 2 or SEQ ID NO:4. The invention provides for a nucleic acid comprising an ASO that binds to the nucleic acid sequence of APOE3, APOE4, or APOE2.

shRNA

The invention also provides for a nucleic acid comprising a nucleic acid expression vector encoding a short hairpin RNA (shRNA), wherein the shRNA comprises an siRNA nucleotide sequence that binds to a human nucleic acid sequence comprising SEQ ID NO: 2 or SEQ ID NO:4. The invention also provides for a nucleic acid comprising a nucleic acid expression vector encoding a shRNA, wherein the shRNA comprises an siRNA nucleotide sequence that binds to a nucleic acid sequence of APOE3, APOE4, or APOE2.

In one aspect of the invention, a host organism comprises a nucleic acid of the invention. In an additional embodiment, the host is a prokaryote or a eukaryote. In another embodiment, a cell comprises a nucleic acid of the invention. In yet another embodiment, a non-human mammal comprises one or more cells provided for by the invention.

Small interfering RNAs can be expressed in vivo in the form of short, fold-back, hairpin loop structures known as short hairpin RNAs (shRNAs) comprising the siRNA sequence of interest. When expressed in a cell, shRNA is rapidly processed by intracellular machinery into siRNA. Expression of shRNAs is accomplished by ligating the shRNA into an expression cassette of a double stranded RNA (dsRNA) expression vector. Expression may be driven by RNA polymerase III promoters (See U.S. Pat. No. 6,852,535). Plasmid vectors for expression of shRNAs are commercially available from vendors such as Gene Therapy Systems, Ambion and Stratagene. U.S. Publication No. 2005/0019918A1 describes the use of a lentiviral vector for in vivo siRNA expression. Methods for DNA and RNA manipulations, including ligation and purification, are well known to those skilled in the art. Vectors comprising shRNA expression cassettes may be introduced into prokaryotic or eukaryotic cells using methods known to one skilled in the art.

Xenograft tumor models are widely used to study human diseases in non-human mammals. To study the impact of protein expression on tumor growth, cells harboring vectors expressing siRNA that specifically inhibits expression of the can be implanted into an immunodeficient mouse under conditions which promote the formation of a tumor consisting of the implanted cells.

Short hairpin RNAs are available through commercial vendors, many vendors also have online algorithms useful for designing shRNAs (i.e., Clontech, ExpressOn, Gene Link and BD Biosciences).

Within the scope of the present invention are nucleic acid molecules, including, but not limited to, siRNA, shRNA, and antisense oligonucleotides, that are specific for APOE4 and/or APOE3 and/or APOE2, and methods of administration to a subject.

PNA

Peptide nucleic acids (PNAs) comprise naturally-occurring DNA bases (i.e., adenine, thymine, cytosine, guanine) or artificial bases (i.e., bromothymine, azaadenines, azaguanines) attached to a peptide backbone through a suitable linker. Nonlimiting examples of PNA backbone linking moieties include amide, thioamide, sulfinamide or sulfonamide linkages. Preferably, the linking moieties in the PNA backbone comprise N-ethylaminoglycine units, and the bases are covalently bound to the PNA backbone by methylene-carbonyl groups. PNAs bind complementary DNA or RNA strands more strongly than a corresponding DNA. They can be utilized in a manner similar to antisense oligonucleotides to block the translation of specific mRNA transcripts. PNA oligomers can be prepared according to the method provided by U.S. Pat. No. 6,713,602. U.S. Pat. No. 6,723,560 describes methods for modulating transcription and translation using sense and antisense PNA oligomers, respectively. Also included in this patent are methods for administration of PNAs to a subject such that the oligomers cross biological barriers and engender a sequence specific response. The PNA can be attached to a targeting moiety, such as an internalization peptide, facilitate uptake of the PNA by cells or tissues.

Within the scope of the present invention are PNAs specific for APOE4 and/or APOE3 and/or APOE2, and methods of administration of PNAs to a subject.

Peptides and Peptidomimetics

In another aspect, a compound of the invention is a peptide or peptidomimetic agonist or antagonist, inhibitor or activator, of APOE4 or APOE3 or APOE2.

Peptides may be synthesized by methods well known in the art, including chemical synthesis and recombinant DNA methods. A peptidomimetic is a compound that is structurally similar to a peptide, such that the peptidomimetic retains the functional characteristics of the peptide. Peptidomimetics include organic compounds and modified peptides that mimic the three-dimensional shape of a peptide. As described in U.S. Pat. No. 5,331,573, the shape of the peptidomimetic may be designed and evaluated using techniques such as NMR or computational techniques. APOE4 and APOE3 and APOE2 inhibitors or activators can be designed based on the structural characteristics of APOE4 and APOE3 and APOE2, respectively. Mutational analyses known in the art may be used to define amino acids or amino acid sequences required for protein-protein interactions.

Within the scope of the present invention are peptide or peptidomimetic inhibitors and activators sharing sufficient homology with and binding to the interaction domains, or portions thereof, which may be used, for example, to block complex formation between APOE4 and its receptors, or between APOE4 and Aβ, or between APOE2 and its receptors, or between APOE2 and Aβ, or enhance the activity of APOE3, or modify their interaction with lipids or cholesterol.

The invention encompasses a composition comprising one or more peptides provided for by the invention and a pharmaceutically acceptable carrier. The invention also encompasses a composition comprising one or more peptidomimetics provided for by the invention and a pharmaceutically acceptable carrier.

In one embodiment, a APOE4 inhibitor, or a APOE4 activator, can be a peptide fragment that binds a protein comprising SEQ ID NO: 1 or 5. For example, the fragment can encompass any portion of at least about 8 consecutive amino acids of SEQ ID NO: 1 or 5. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 1 or 5. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England).

In one embodiment, a APOE3 activator, or a APOE3 inhibitor, can be a peptide fragment that binds a protein comprising SEQ ID NO: 3 or 6. For example, the fragment can encompass any portion of at least about 8 consecutive amino acids of SEQ ID NO: 3 or 6. The fragment can comprise at least about 10 consecutive amino acids, at least about 20 consecutive amino acids, at least about 30 consecutive amino acids, at least about 40 consecutive amino acids, at least about 50 consecutive amino acids, at least about 60 consecutive amino acids, or at least about 75 consecutive amino acids of SEQ ID NO: 3 or 6. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England).

Antibodies

In one aspect, a compound of the invention is an antibody agonist or antagonist, inhibitor or activator, or a fragment thereof, of APOE4 or APOE3 or APOE2.

APOE4-specific antibodies are commercially available and include, but are not limited to, ApoE4 Antibody (BioVision), Apolipoprotein E4 antibody (MBL International), Apo-E4 (5G7) monoclonal antibody (Covance), Apo-E4 (9D11) monoclonal antibody (Covance), and ApoE4 (5B5) anti-human mouse IgG MoAb (IBL-America (Immuno-Biological Laboratories)).

The invention provides for an antibody, or antigen-binding fragment thereof, that specifically binds to the APOPE4 protein, or the APOE3 protein or the APOE2 protein. Within the context of the invention, the antibody, or fragment thereof, can be monoclonal, polyclonal, chimeric or humanized. Such antibodies and antigen-binding fragments may be used, for example, to block complex formation between APOE4 and its receptors, or between APOE4 and Aβ, or between APOE2 and its receptors, or between APOE2 and Aβ, or enhance the activity of APOE3, or modify their interaction with lipids or cholesterol.

A compound of the invention can be a protein, such as an antibody (monoclonal, polyclonal, humanized, chimeric, or fully human), or a binding fragment thereof, directed against a polypeptide encoded by SEQ ID NO: 1, 3, 5 or 6. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

The invention also encompasses use of the antibodies provided by the invention for diagnostic or therapeutic purposes. For example, the antibodies may be used for staining human Alzheimer's Disease specimens to diagnose. The antibodies may also be used, for example, for discriminating between early-onset Alzheimer's Disease and late-onset Alzheimer's Disease, and familial Alzheimer's Disease, and used as a research tool to elucidate the molecular mechanisms involved in the APOE4 pathway.

The invention encompasses a composition comprising one or more antibodies provided for by the invention and a pharmaceutically acceptable carrier. The invention also encompasses a composition comprising one or more hybridoma cells provided for by the invention and a pharmaceutically acceptable carrier.

Small Molecules

In another aspect of the invention, a compound of the invention is a small molecule inhibitor, antagonist, activator, or agonist of APOE4, capable of blocking or enhancing APOE4 expression or binding, or a small molecule inhibitor, antagonist, activator, or agonist, of APOE3, capable of blocking or enhancing APOE3 expression or binding, or a small molecule inhibitor, antagonist, activator, or agonist, of APOE2, capable of blocking or enhancing APOE2 expression or binding. Within the scope of the invention, the small molecule comprises an organic molecule. Also within the scope of the invention, the small molecule comprises an inorganic molecule. Protein-protein interaction inhibitors may act directly via inhibition at the protein-protein interface, or indirectly via binding to a site not at the interface and inducing a conformational change in the protein such that the protein is prohibited from engaging in the protein-protein interaction (Pagliaro et al., Curr Opin Chem Biol 8:442-449 (2004)). U.S. Publication No. 2005/0032245A1 describes methods for determining such inhibitors and evaluating potential inhibitors that prevent or inhibit protein-protein interactions.

A compound of the invention can also be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries (Werner et al., (2006) Brief Funct. Genomic Proteomic 5(1):32-6). In some embodiments, the agent is a small molecule that binds, interacts, or associates with a target protein or RNA. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

Small molecule inhibitors of APOE4 can include, but are not limited to Congo Red, X-34, curcumin and CB9032258.

Small molecule activators of APOE3 or APOE4 or APOE2 include bexarotene, which induces expression of the proteins.

Additional examples for determining inhibitors or antagonists, activators or agonists of APOE4 or APOE3 or APOE2 use the protein crystal structure of APOE4 and APOE3 and APOE2, respectively. The crystal structure of APOE4 and APOE3 and APOE2 may be used to screen for APOE4 and APOE3 and APOE2 inhibitors or antagonists, activators or agonists or to design APOE4 and APOE3 and APOE2 inhibitors or antagonists, activators or agonists, respectively. One of ordinary skill in the art can solve the crystal structure of APOE4 and APOE3 and APOE2 and determine sites which confer APOE4 and APOE3 and APOE2 function, respectively. Based on the crystal structure, in silico screens of compound databases may be performed to discover compounds that would be predicted to inhibit or activate APOE4 or APOE3 or APOE2. These compounds can then be evaluated in assays to determine if they inhibit or activate APOE4 or APOE3 or APOE2 function. Additionally, the crystal structure can be used to design compounds (i.e., rational drug design) that would be predicted to inhibit or activate APOE4 or APOE3 or APOE2 function based on the structure of the compound, then the compound can be tested in assays to determine if they inhibit or activate APOE4 or APOE3 or APOE2 function, respectively.

One of skill in the art will understand that other agents may be useful as agonist or antagonist, inhibitor or activator, of APOE4 or APOE3 or APOE2, and may be used in conjunction with the methods of the invention.

### Pharmaceutical Compositions and Administration for Therapy

Compounds of the invention can be administered to the subject once (e.g., as a single injection or deposition). Alternatively, compounds of the invention can be administered once or twice daily to a subject in need thereof for a period of from about two to about twenty-eight days, or from about seven to about ten days. Compounds of the invention can also be administered once or twice daily to a subject for a period of 1, 2, 3, 4, 5, 6, 7, 8, 9, 10, 11, 12 times per year, or a combination thereof. Furthermore, compounds of the invention can be co-administrated with another therapeutic. Where a dosage regimen comprises multiple administrations, the effective amount of the compound(s) administered to the subject can comprise the total amount of the compound(s) administered over the entire dosage regimen.

Compounds can be administered to a subject by any means suitable for delivering the compounds to cells of the subject, such as brain tissue or neuronal cells. For example, compounds can be administered by methods suitable to transfect cells. Transfection methods for eukaryotic cells are well known in the art, and include direct injection of a nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with a neurodegenerative disorder by any means that produces contact of the active ingredient with the agent's site of action in the body of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

The compounds of the invention may be administered to a subject in an amount effective to treat or prevent a neurodegenerative disorder. One of skill in the art can readily determine what will be an effective amount of the compounds of the invention to be administered to a subject, taking into account whether the compound is being used prophylactically or therapeutically, and taking into account other factors such as the age, weight and sex of the subject, any other drugs that the subject may be taking, any allergies or contraindications that the subject may have, and the like. For example, an effective amount can be determined by the skilled artisan using known procedures, including analysis of titration curves established in vitro or in vivo. Also, one of skill in the art can determine the effective dose from performing pilot experiments in suitable animal model species and scaling the doses up or down depending on the subjects weight etc. Effective amounts can also be determined by performing clinical trials in individuals of the same species as the subject, for example starting at a low dose and gradually increasing the dose and monitoring the effects on a neurodegenerative disorder. Appropriate dosing regimens can also be determined by one of skill in the art without undue experimentation, in order to determine, for example, whether to administer the agent in one single dose or in multiple doses, and in the case of multiple doses, to determine an effective interval between doses.

A therapeutically effective dose of a compound that treats or prevents a neurodegenerative disorder can depend upon a number of factors known to those of ordinary skill in the art. The dose(s) of the compounds can vary, for example, depending upon the identity, size, and condition of the subject or sample being treated, further depending upon the route by which the composition is to be administered, if applicable, and the effect which the practitioner desires the compound to have upon the target of interest. These amounts can be readily determined by a skilled artisan. These amounts include, for example, mg or microgram (μg) amounts per kilogram (kg) of subject weight, such as about 0.25 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg, or between about 0.25 mg/kg to 0.5 mg/kg, 0.5 mg/kg to 1 mg/kg, 1 mg/kg to 2 mg/kg, 2 mg/kg to 3 mg/kg, 3 mg/kg to 4 mg/kg, 4 mg/kg to 5 mg/kg, 5 mg/kg to 6 mg/kg, 6 mg/kg to 7 mg/kg, 7 mg/kg to 8 mg/kg, 8 mg/kg to 9 mg/kg, or 9 mg/kg to 10 mg/kg, or any range in between. These amounts also include a unit dose of a compound, for example, mg or μg amounts, such as at least about 0.25 mg, 0.5 mg, 1 mg, 2 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 750 mg, 800 mg, 850 mg, 900 mg, or more. Any of the therapeutic applications described herein can be applied to any subject in need of such therapy, including, for example, a mammal such as a dog, a cat, a cow, a horse, a rabbit, a monkey, a pig, a sheep, a goat, or a human.

For compounds that are antagonists or inhibitors, or agonists, or activators, of a APOE3 or APOE4 or APOE2 protein, or compounds that increase or decrease the expression of the APOE3 or APOE4 or APOE2 genes, the instructions would specify use of the pharmaceutical composition for treating or preventing a neurodegenerative disorder.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remmington's Pharmaceutical Sciences, Meade Publishing Co., Easton, Pa. (20^{th} Ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The invention also provides for a kit that comprises a pharmaceutically acceptable carrier and a compound identified using the screening assays of the invention packaged with instructions for use.

A pharmaceutical composition containing a compound of the invention can be administered in conjunction with a pharmaceutically acceptable carrier, for any of the therapeutic effects discussed herein. Such pharmaceutical compositions can comprise, for example antibodies directed to polypeptides encoded by genes of interest or variants thereof, or agonists and antagonists of a polypeptide encoded by a gene of interest. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous applications can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of injectable compositions can be brought about by incorporating an agent which delays absorption, for example, aluminum monostearate and gelatin.

Sterile injectable solutions can be prepared by incorporating the compound (e.g., a small molecule, peptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. In some embodiments, the compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Administration of the compound is not restricted to a single route, but may encompass administration by multiple routes. For instance, exemplary administrations by multiple routes include, among others, a combination of intradermal and intramuscular administration, or intradermal and subcutaneous administration. Multiple administrations may be sequential or concurrent. Other modes of application by multiple routes will be apparent to the skilled artisan.

The compounds of the invention may be formulated into compositions for administration to subjects for the treatment and/or prevention of a neurodgenerative disorder. Such compositions may comprise the compounds of the invention in admixture with one or more pharmaceutically acceptable diluents and/or carriers and optionally one or more other pharmaceutically acceptable additives. The pharmaceutically-acceptable diluents and/or carriers and any other additives must be “acceptable” in the sense of being compatible with the other ingredients of the composition and not deleterious to the subject to whom the composition will be administered. One of skill in the art can readily formulate the compounds of the invention into compositions suitable for administration to subjects, such as human subjects, for example using the teaching a standard text such as Remington's Pharmaceutical Sciences, 18th ed, (Mack Publishing Company: Easton, Pa., 1990), pp. 1635-36), and by taking into account the selected route of delivery.

Examples of diluents and/or carriers and/or other additives that may be used include, but are not limited to, water, glycols, oils, alcohols, aqueous solvents, organic solvents, DMSO, saline solutions, physiological buffer solutions, peptide carriers, starches, sugars, preservatives, antioxidants, coloring agents, pH buffering agents, granulating agents, lubricants, binders, disintegrating agents, emulsifiers, binders, excipients, extenders, glidants, solubilizers, stabilizers, surface active agents, suspending agents, tonicity agents, viscosity-altering agents, carboxymethyl cellulose, crystalline cellulose, glycerin, gum arabic, lactose, magnesium stearate, methyl cellulose, powders, saline, sodium alginate. The combination of diluents and/or carriers and/or other additives used can be varied taking into account the nature of the active agents used (for example the solubility and stability of the active agents), the route of delivery (e.g. oral, parenteral, etc.), whether the agents are to be delivered over an extended period (such as from a controlled-release capsule), whether the agents are to be co-administered with other agents, and various other factors. One of skill in the art will readily be able to formulate the compounds for the desired use without undue experimentation.

The compounds of the invention may be administered to a subject by any suitable method that allows the agent to exert its effect on the subject in vivo. For example, the compositions may be administered to the subject by known procedures including, but not limitated to, by oral administration, sublingual or buccal administration, parenteral administration, transdermal administration, via inhalation, via nasal delivery, vaginally, rectally, and intramuscularly. The compounds of the invention may be administered parenterally, or by epifascial, intracapsular, intracutaneous, subcutaneous, intradermal, intrathecal, intramuscular, intraperitoneal, intrasternal, intravascular, intravenous, parenchymatous, or sublingual delivery. Delivery may be by injection, infusion, catheter delivery, or some other means, such as by tablet or spray. In one embodiment, the compounds of the invention are administered to the subject by way of delivery directly to the brain tissue, such as by way of a catheter inserted into, or in the proximity of the subject's brain, or by using delivery vehicles capable of targeting the drug to the brain. For example, the compounds of the invention may be conjugated to or administered in conjunction with an agent that is targeted to the brain, or the spinal cord, such as an antibody or antibody fragment. In one embodiment, the compounds of the invention are administered to the subject by way of delivery directly to the tissue of interest, such as by way of a catheter inserted into, or in the proximity of the subject's tissue of interest, or by using delivery vehicles capable of targeting the drug to the brain, or the spinal cord, such as an antibody or antibody fragment.

For oral administration, a formulation of the compounds of the invention may be presented as capsules, tablets, powders, granules, or as a suspension or solution. The formulation may contain conventional additives, such as lactose, mannitol, cornstarch or potato starch, binders, crystalline cellulose, cellulose derivatives, acacia, cornstarch, gelatins, disintegrators, potato starch, sodium carboxymethylcellulose, dibasic calcium phosphate, anhydrous or sodium starch glycolate, lubricants, and/or or magnesium stearate.

For parenteral administration (i.e., administration by through a route other than the alimentary canal), the compounds of the invention may be combined with a sterile aqueous solution that is isotonic with the blood of the subject. Such a formulation may be prepared by dissolving the active ingredient in water containing physiologically-compatible substances, such as sodium chloride, glycine and the like, and having a buffered pH compatible with physiological conditions, so as to produce an aqueous solution, then rendering the solution sterile. The formulation may be presented in unit or multi-dose containers, such as sealed ampoules or vials. The formulation may be delivered by injection, infusion, or other means known in the art.

For transdermal administration, the compounds of the invention may be combined with skin penetration enhancers, such as propylene glycol, polyethylene glycol, isopropanol, ethanol, oleic acid, N-methylpyrrolidone and the like, which increase the permeability of the skin to the compounds of the invention and permit the compounds to penetrate through the skin and into the bloodstream. The compounds of the invention also may be further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, the compounds of the invention are provided in unit dose form such as a tablet, capsule or single-dose injection or infusion vial.

Various routes of administration and various sites of cell implantation can be utilized, such as, subcutaneous, intramuscular, or in brain tissue, or neuronal tissue, in order to introduce aggregated population of cells into a site of preference. Once implanted in a subject (such as a mouse, rat, or human), the aggregated cells can then treat or prevent a neurodegenerative disorder within the subject. In one embodiment, transfected cells (for example, cells expressing a protein encoded by a APOE4 or APOE3 or APOE2 gene) are implanted in a subject to treat or prevent a neurodegenerative disorder within the subject. In other embodiments, the transfected cells are cells derived from brain tissue. In further embodiments, the transfected cells are neuronal cells. Aggregated cells (for example, cells grown in a hanging drop culture) or transfected cells (for example, cells produced as described herein) maintained for 1 or more passages can be introduced (or implanted) into a subject (such as a rat, mouse, dog, cat, human, and the like).

“Subcutaneous” administration can refer to administration just beneath the skin (i e, beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration.

Administration of the cell aggregates is not restricted to a single route, but can encompass administration by multiple routes. For instance, exemplary administrations by multiple routes include, among others, a combination of intradermal and intramuscular administration, or intradermal and subcutaneous administration. Multiple administrations can be sequential or concurrent. Other modes of application by multiple routes will be apparent to the skilled artisan.

In other embodiments, this implantation method will be a one-time treatment for some subjects. In further embodiments of the invention, multiple cell therapy implantations will be required. In some embodiments, the cells used for implantation will generally be subject-specific genetically engineered cells. In another embodiment, cells obtained from a different species or another individual of the same species can be used. Thus, using such cells can require administering an immunosuppressant to prevent rejection of the implanted cells. Such methods have also been described in United States Patent Application Publication 2004/0057937 and PCT application publication WO 2001/32840, and are hereby incorporated by reference.

Gene Therapy and Protein Replacement Methods

Delivery of nucleic acids into viable cells can be effected ex vivo, in situ, or in vivo by use of vectors, such as viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (See, for example, Anderson, Nature, supplement to vol. 392, no. 6679, pp. 25-20 (1998)). Introduction of a nucleic acid or a gene encoding a polypeptide of the invention can also be accomplished with extrachromosomal substrates (transient expression) or artificial chromosomes (stable expression). Cells can also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., 1992), adenovirus (Berkner, 1992; Berkner et al., 1988; Gorziglia and Kapikian, 1992; Quantin et al., 1992; Rosenfeld et al., 1992; Wilkinson et al., 1992; Stratford-Perricaudet et al., 1990), vaccinia virus (Moss, 1992), adeno-associated virus (Muzyczka, 1992; Ohi et al., 1990), herpesviruses including HSV and EBV (Margolskee, 1992; Johnson et al., 1992; Fink et al., 1992; Breakfield and Geller, 1987; Freese et al., 1990), and retroviruses of avian (Biandyopadhyay and Temin, 1984; Petropoulos et al., 1992), murine (Miller, 1992; Miller et al., 1985; Sorge et al., 1984; Mann and Baltimore, 1985; Miller et al., 1988), and human origin (Shimada et al., 1991; Helseth et al., 1990; Page et al., 1990; Buchschacher and Panganiban, 1992). Non-limiting examples of in vivo gene transfer techniques include transfection with viral (e.g., retroviral) vectors (see U.S. Pat. No. 5,252,479, which is incorporated by reference in its entirety) and viral coat protein-liposome mediated transfection (Dzau et al., Trends in Biotechnology 11:205-210 (1993), incorporated entirely by reference). For example, naked DNA vaccines are generally known in the art; see Brower, Nature Biotechnology, 16:1304-1305 (1998), which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al., Science 256:808-813 (1992); U.S. Pat. Nos. 5,252,479, 5,747,469, 6,017,524, 6,143,290, 6,410,010 6,511,847; and U.S. Application Publication Nos. 2002/0077313 and 2002/00069, which are all hereby incorporated by reference in their entireties. For additional reviews of gene therapy technology, see Friedmann, Science, 244:1275-1281 (1989); Verma, Scientific American: 68-84 (1990); Miller, Nature, 357: 455-460 (1992); Kikuchi et al., J Dermatol Sci. 2008 May;50(2):87-98; Isaka et al., Expert Opin Drug Deliv. 2007 September; 4(5):561-71; Jager et al., Curr Gene Ther. 2007 August; 7(4):272-83; Waehler et al., Nat Rev Genet. 2007 August; 8(8):573-87; Jensen et al., Ann Med. 2007; 39(2):108-15; Herweijer et al., Gene Ther. 2007 January; 14(2):99-107; Eliyahu et al., Molecules, 2005 Jan. 31; 10(1):34-64; and Altaras et al., Adv Biochem Eng Biotechnol. 2005; 99:193-260, all of which are hereby incorporated by reference in their entireties.

Protein replacement therapy can increase the amount of protein by exogenously introducing wild-type or biologically functional protein by way of infusion. A replacement polypeptide can be synthesized according to known chemical techniques or can be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

A polypeptide encoded by a gene of interest, for example, but not limited to, APOE4 or APOE3 or APOE2, can also be delivered in a controlled release system. For example, the polypeptide can be administered using intravenous infusion, an implantable osmotic pump, a transdermal patch, liposomes, or other modes of administration. In one embodiment, a pump can be used (see is Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

### Combination Therapy

According to the methods of the invention, a compound of the invention can be administered to a subject either as a single agent, or in combination with one or more other agents. In one embodiment, a compound of the invention is administered to a subject as a single agent. In one embodiment, a compound of the invention is administered to a subject alone. In one embodiment, a compound of the invention is administered to a subject in combination with one or more other agents.

In certain embodiments, a compound of the invention may be used in combination with other agents that are used for the treatment or prevention of a neurodegenerative disorder. In certain embodiments, a compound of the invention may be used in combination with other agents that are not used for the treatment or prevention of a neurodegenerative disorder. In one embodiment, a compound of the invention may be delivered to a subject as part of the same pharmaceutical composition or formulation containing one or more additional active agents. In another embodiment, a compound of the invention may be delivered to a subject in a composition or formulation containing only that active agent, while one or more other agents are administered to the subject in one or more separate compositions or formulations. In one embodiment, the other agents are not used for the treatment or prevention of a neurodegenerative disorder. In another embodiment, the other agents are used for the treatment or prevention of a neurodegenerative disorder.

A compound of the invention and the other agents that are used for the treatment or prevention of a neurodegenerative disorder may be administered to the subject at the same time, or at different times. A compound of the invention and the other agents that are not used for the treatment or prevention of a neurodegenerative disorder may be administered to the subject at the same time, or at different times. For example, a compound of the invention and the other agents may be administered within minutes, hours, days, weeks, or months of each other, for example as part of the overall treatment regimen of a subject. In some embodiments, a compound of the invention may be administered prior to the administration of other agents. In other embodiments, a compound of the invention may be administered subsequent to the administration of other agents.

A compound of the invention may also be used in combination with known therapies for a neurodegenerative disorder. A compound of the invention may also be used in combination with surgical or other interventional treatment regimens used for the treatment of a neurodegenerative disorder.

Compounds of the invention, as described above, including, but not limited to, inhibitors, activators, agonists and antagonists of APOE4, APOE3 or APOE2, may be used in combination with each other for the treatment or prevention of a neurodegenerative disorder.

In one embodiment, the neurodegenerative disorder is Alzheimer's Disease. In another embodiment, the neurodegenerative disorder is Familial Alzheimer's Disease (FAD). In another embodiment, the neurodegenerative disorder is Sporadic Alzheimer's Disease. In one embodiment, the neurodegenerative disorder is Early-Onset Alzheimer's Disease. In another embodiment, the neurodegenerative disorder is Late-Onset Alzheimer's Disease (LOAD).

In one embodiment, the neurodegenerative disorder is dementia. In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease.

In some embodiments, the administration of a compound of the invention in combination with one or more other agents has an additive effect, in comparison with administration of the compound of the invention alone, or administration of the one or more other agents alone. In other embodiments, the administration of a compound of the invention in combination with one or more other agents has a synergistic effect, in comparison with administration of the compound of the invention alone, or administration of the one or more other agents alone. In some embodiments, the administration of a compound of the invention in combination with one or more other agents can help reduce side effects, in comparison with administration of the compound of the invention alone, or administration of the one or more other agents alone.

In some embodiments, the compound of the invention is used as an adjuvant therapy. In other embodiments, the compound of the invention is used in combination with an adjuvant therapy.

### Subjects

According to the methods of the invention, the subject or patient can be any animal that has or is diagnosed with a neurodegenerative disorder. According to the methods of the invention, the subject or patient can be any animal that is predisposed to or is at risk of developing a neurodegenerative disorder. In preferred embodiments, the subject is a human subject. In some embodiments, the subject is a rodent, such as a mouse. In some embodiments, the subject is a cow, pig, sheep, goat, cat, horse, dog, and/or any other species of animal used as livestock or kept as pets.

In some embodiments, the subject is already suspected to have a neurodegenerative disorder. In other embodiments, the subject is being treated for a neurodegenerative disorder, before being treated according to the methods of the invention. In other embodiments, the subject is not being treated for a neurodegenerative disorder, before being treated according to the methods of the invention.

In one embodiment, the neurodegenerative disorder is Alzheimer's Disease. In another embodiment, the neurodegenerative disorder is Familial Alzheimer's Disease (FAD). In another embodiment, the neurodegenerative disorder is Sporadic Alzheimer's Disease. In one embodiment, the neurodegenerative disorder is Early-Onset Alzheimer's Disease. In another embodiment, the neurodegenerative disorder is Late-Onset Alzheimer's Disease (LOAD).

In one embodiment, the neurodegenerative disorder is multiple sclerosis. In another embodiment, the neurodegenerative disorder is traumatic brain injury. In one embodiment, the neurodegenerative disorder is subarachnoid hemorrhage. In another embodiment, the neurodegenerative disorder is stroke. In one embodiment, the neurodegenerative disorder is dementia puglistica. In another embodiment, the neurodegenerative disorder is Parkinson's disease. In one embodiment, the neurodegenerative disorder is a cognitive disorder. In one embodiment, the neurodegenerative disorder is mild cognitive disorder. In another embodiment, the neurodegenerative disorder is frontotemporal dementia.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

There has been significant interest in the generation and analysis of human neuron models of Alzheimer's disease (AD). A major driving force for such interest is the application of human neuron models to ‘sporadic’ disease mechanisms. Here, Sporadic AD was pursued using the hiN cell conversion technology. Both the role of the major AD risk allele, APOE4, as well as the relevance of disease status (AD or unaffected), were investigated. A robust and consistent effect of the risk-associated variant, APOE4, on APP processing and APP endosomal trafficking was found. APOE4 carrier derived hiN cultures display elevated levels of both Abeta42 and Abeta40 as a consequence of increased BACE activity towards APP. Consistent with this, APP is preferentially localized to early endosomal compartments that harbor BACE1. In contrast to the impact of APOE4 on APP processing and trafficking, a consistent effect of disease status (AD versus unaffected) on these phentoypes was not observed. The lack of a clear effect of disease status is reminiscent of Israel et al., 2012, Probing sporadic and familial Alzheimer's disease using induced pluripotent stem cells, Nature, 482(7384):216-20 (which failed to observe a consistent effect as well), and indicates that ‘sporadic’ disease is somewhat heterogeneous. The most significant aspect of the present study is that “rescue” of both APP processing and APP trafficking phenotypes in APOE4 carrier hiN cells by protein complementation is observed. An antibody selective for APOE4 is shown to suppress the APP processing and localization phenotypes in APOE4 carriers, as does excess APOE3 protein. In contrast, addition of APOE4 protein to APOE3/3 cultures leads to a phenocopy of the APOE4 carrier hiNs. These findings not only validate the role of APOE in the observed phenotypes, but also point to a therapeutic strategy.

Finally, the endocytosis kinetics in the APOE4 hiNs were probed more directly. First, it is shown that the APOE4 cells harbor a general increase in the total area of early endosomes, quantified by EEA1 staining, as well as late endosomes/lysosomes, quantified by LAMP2 staining Consistent with this, receptor mediated endocytosis of Transferrin (a classical marker assay for trafficking from the cell surface to early endosomes) is potentiated.

The phenotype of the APOE4 carriers is somewhat reminiscent of the FAD PSEN hiNs previously reported, which is unexpected, but there are important distinctions nonetheless. For instance, in terms of APP processing, the effect of APOE4 in hiN cultures is to modify BACE1 cleavage; in contrast, PSEN mutant hiN cultures show altered γ-Secretase activity in terms of the Abeta42:40 ratio, but not altered BACE1 activity towards APP.

Most APOE4 carriers do not ultimately develop AD. The presence of such phenotypes in unaffected APOE4 carrier hiNs supports a model wherein the APOE4 allele leads to a highly penetrant predisease state, that in a small cohort of individuals converts to AD (presumably due to secondary insults).

Without being bound by theory, APOE is associated with AD because of a role for APOE in Aβ clearance from brain parenchyma. However, other studies have favored a more direct role for APOE4 on neurons, either in terms of APP processing or by way of mechanisms independent of A13.

### Example 2

### Human Induced Neurons from APOE4 Carriers Display Endosomal Missorting and Altered Processing of APP: Evidence of a Pre-Alzheimer's Disease State

The numbers between parentheses in this Example refer to the numbered references in the list of references that follows this Example.

Non-familial late-onset Alzheimer's disease (LOAD) can be the consequence of interacting genetic and environmental risk factors (1-6), ultimately leading to a selective neurodegeneration with cognitive decline. The apolipoprotein ε4 (APOE4) allele represents the most important known genetic risk factor for LOAD (7): harboring a single APOE4 allele increases disease risk over 3-fold, whereas homozygosity for APOE4 increases risk over 10-fold (8). APOE4 can play a role in multiple mechanisms in LOAD pathology (9, 10, 11), including reduced clearance of the amyloid precursor protein-derived (APP) Aβ fragment that is typically accumulated in LOAD brain (12), altered APP proteolytic processing (13, 14, 15), as well as APP-independent aspects of neuronal survival (16) and function (9, 17). To pursue cellular mechanisms of LOAD and the role of APOE4 therein, human induced neurons (hiNs) were generated by the directed conversion of skin fibroblast cultures. hiNs carrying an APOE4 allele displayed significantly increased processing of APP to the Aβ fragment, relative to hiNs from noncarriers, independent of disease status. Analysis of APP processing in APOE4 hiNs revealed increased β-secretase cleavage, the first enzymatic step in Aβ production. Furthermore, APP appeared preferentially localized to endosomal compartments, where the β-secretase-1 (BACE1) enzyme is most active, and away from the cell surface. Consistent with this, APOE4 hiNs displayed potentiated receptor-mediated endocytosis. Taken together, these findings describe a prodromal state in APOE4 hiNs that portends LOAD pathology, and implicate altered endosomal sorting as a common cellular mechanism for the familial (18) and sporadic Alzheimer's disease forms.

A challenge to the mechanistic analysis of LOAD and LOAD-associated risk factors is the paucity of suitable human neuronal model systems. Direct examination of patient brain tissue in the context of slowly progressive neurodegenerative disorders, such as LOAD, can be fraught with difficulty in distinguishing primary etiological findings from late-stage alterations secondary to the disease process. An approach to the analysis of familial AD patient neurons through the directed conversion of skin fibroblasts to hiNs by transduction of a cocktail of neurogenic transcription regulators was recently described (18, 19, 20, 21). To apply this technology to LOAD and APOE4-associated LOAD risk, a panel of human skin fibroblasts was assembled that included samples from 3 unaffected individuals homozygous for the common APOE3 allele (E3/3), 3 unaffected individuals heterozygous for the APOE4 allele (E3/4), and 3 LOAD patients heterozygous for APOE4 (E3/4) (Table 1).

TABLE 1
Summary of individual hiN cell cultures and corresponding
skin fibroblast of origin. All skin fibroblast lines were
derived from de-identified, banked tissue samples; there
was no interaction with subjects, no intervention, and
private, identifiable information was not collected. Details
of cultures are available at http://ccr.coriell.org/. Diagnosis
is based on clinical diagnosis.

| | Culture | Origin |
|---|---|---|
| UND (E3/3) | STC0022 | 65 yo female |
| | AG07871 | 49 yo female |
| | AG07926 | Spouse^{a} female |
| UND (E3/4) | T-4560 | 89+ yo male |
| | AG07619 | 68 yo male |
| | AG07627 | 49 yo female |
| LOAD (E3/4) | STC0033 | 81 yo male |
| | AG06263 | 67 yo female |
| | AG06264 | 62 yo female |

^{a}Culture was derived from spouse of an AD patient, precise age data unavailable.

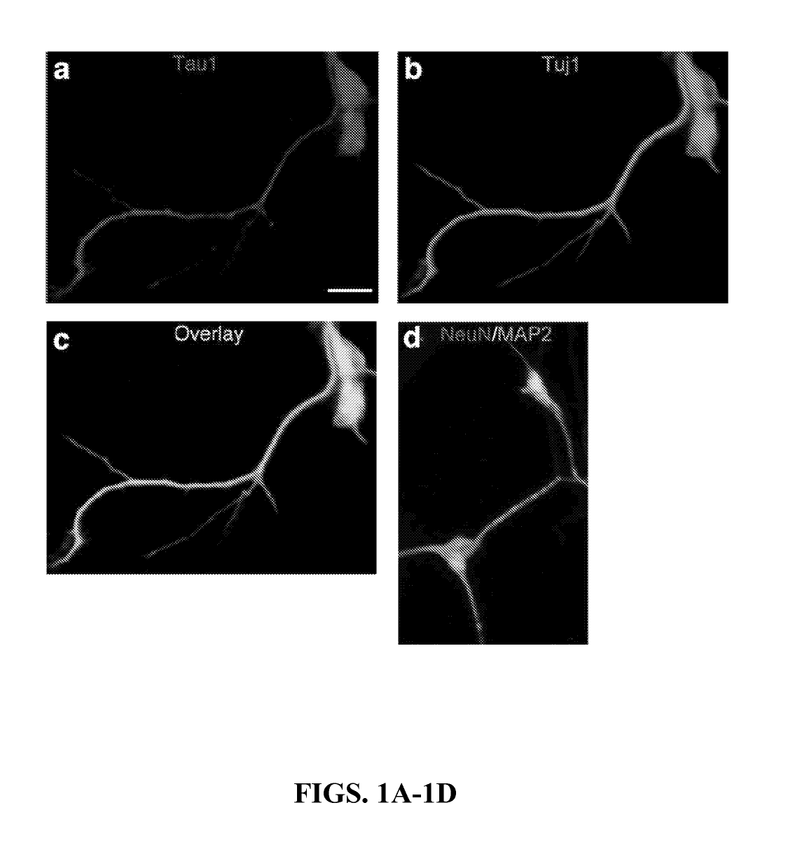

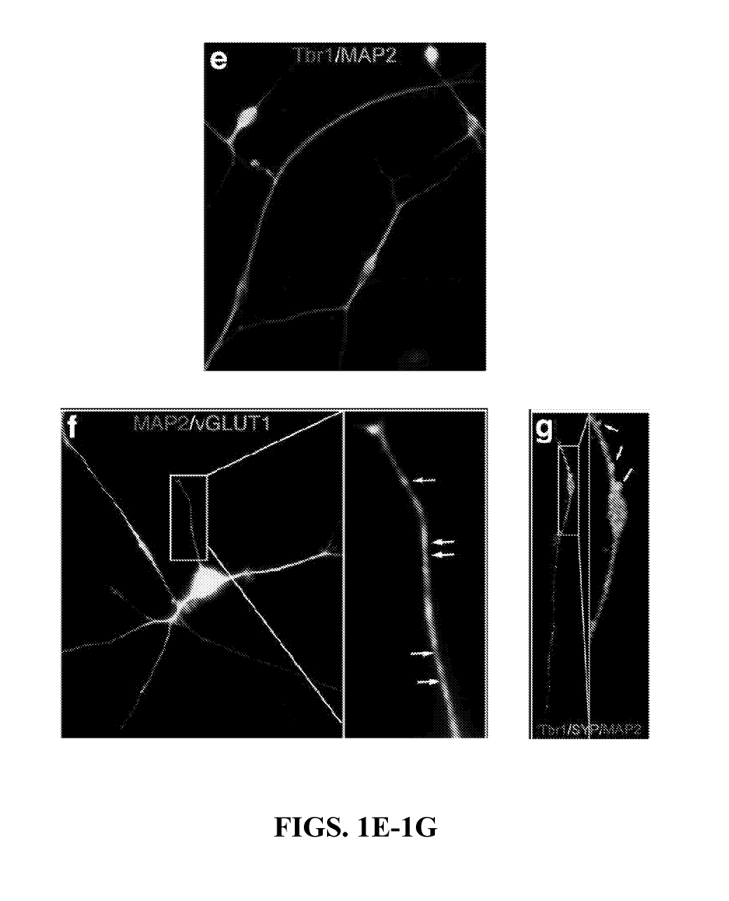

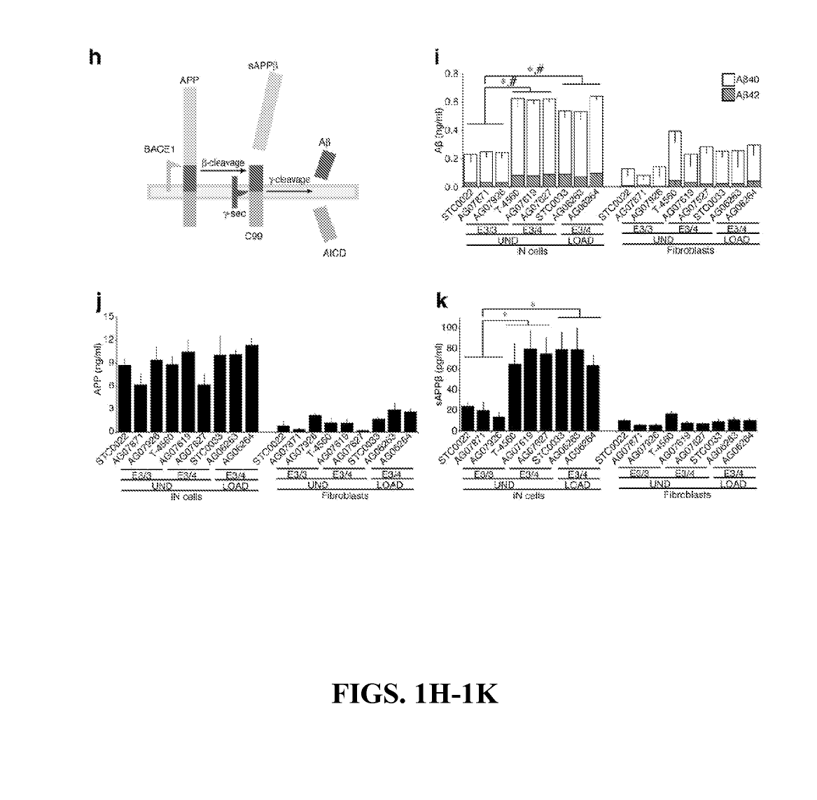

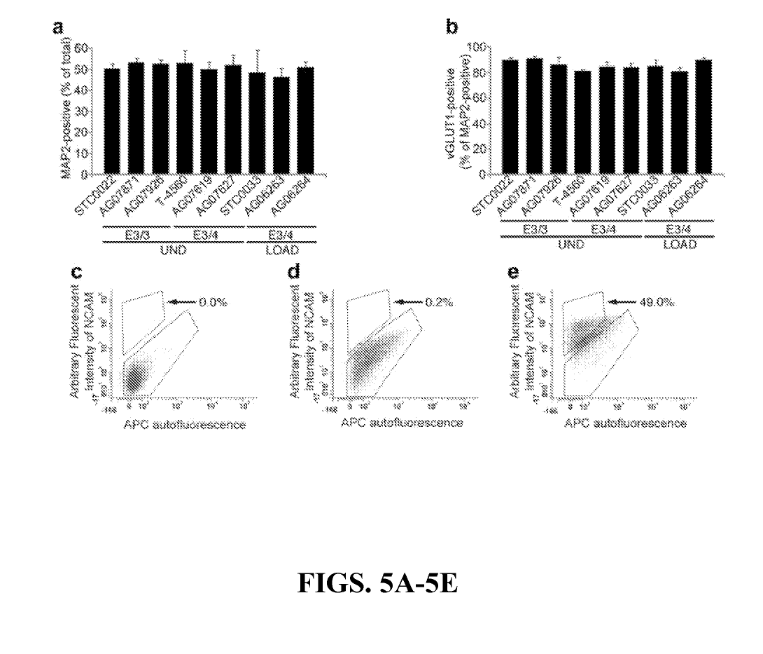

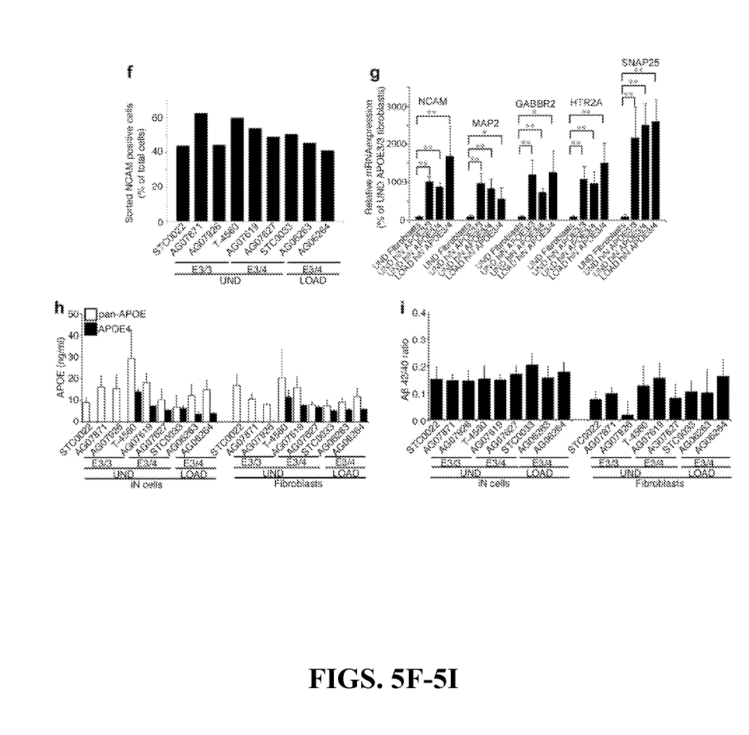

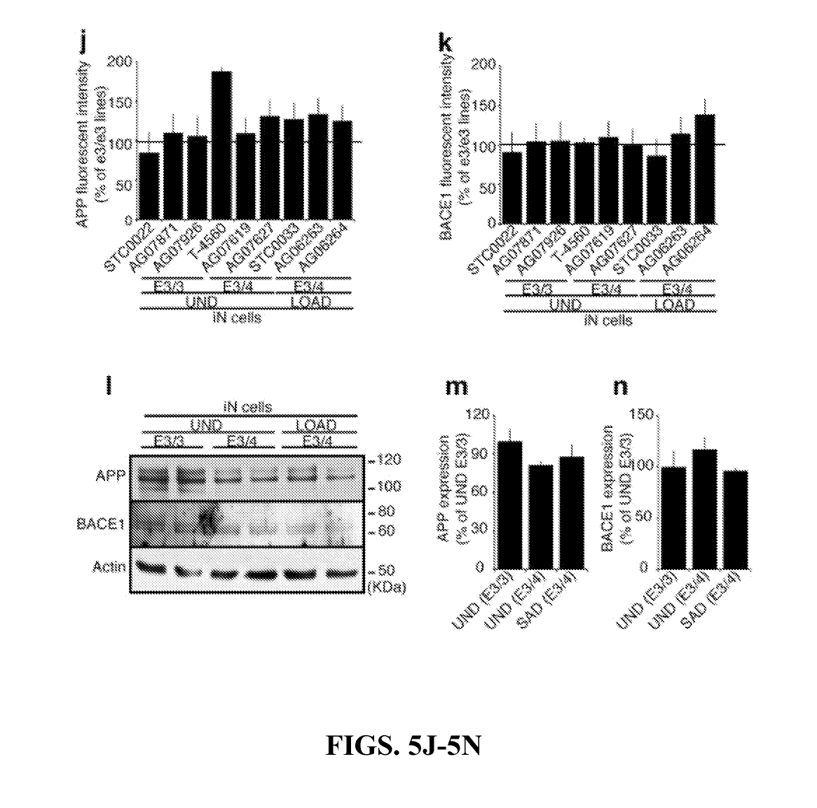

hiN conversion was achieved by lentiviral transduction of the Ascl1, Brn2, Zic1, and Mytl1 (ABZM) cocktail as previously described (18). At 3 weeks after transduction, approximately 50% of the cells in culture displayed a neuronal morphology (with extended neurite processes >2-fold longer than the soma diameter) (18) and expressed general neuronal markers including Tau, Tuj1, NeuN, and MAP2 (FIGS. 1A-G, FIG. 5A). Furthermore, the majority of neuronal cells displayed markers characteristic of forebrain (T-box brain 1; Tbr1; FIG. 1E, G) and glutamatergic (vesicular glutamate transporter; vGLUT1) neurons (FIG. 1F, FIG. 5B). Upon co-culture with astroglia (18), staining with a presynaptic protein marker, synaptophysin, was apparent along neurite processes (FIG. 1G). The 3 groups did not differ significantly in these neuronal characteristics (FIGS. 5A, B). Remaining non-neuronal cells expressed the fibroblast specific protein marker FSP-1 (18). Fluorescence activated cell sorting (FACS) for NCAM, a cell surface marker of neurons and their progenitors, demonstrated comparable neuron numbers across the cultures (FIG. 5F). Gene expression analysis of the FACS purified NCAM positive population by quantitative rt-PCR of cDNA confirmed comparable expression of neuronal genes, such as the gamma-aminobutyric acid B receptor 2 (GABBR2) and the 5-hydroxytryptamine receptor 2A (HTR2A), across all cultures (FIG. 5G). Quantification of APOE4 protein, as well as total APOE protein, in the hiN cell cultures by ELISA of cell media, validated the presence of APOE4 protein only in the E3/4 cultures (FIG. 5H). Furthermore, overall levels of total APOE protein in the media did not differ significantly across the panel of samples or in the context of conversion from fibroblasts to hiNs. hiN cells displayed characteristic neuronal membrane properties and typical neuronal Na+, K+, and Ca2+ channels, as assessed by patch-clamp recording and consistent with prior studies (18, 19, 20, 21).

APP processing to the Aβ fragment was next quantified (FIG. 1H) in the panel of hiN cultures and parental fibroblasts. Strikingly, E3/4 hiN cell cultures—from either unaffected or affected individuals—harbored consistently increased levels of both Aβ40 and Aβ42 forms relative to E3/3 hiN cultures, as determined by ELISA analysis of media (FIG. 1I). Thus, presence of the APOE4 haplotype but not disease status appeared to correlate with altered Aβ accumulation. Parental fibroblast cultures consistently produced lower levels of Aβ then converted hiN cultures, as previously reported (18). Among the unconverted fibroblast cultures from the different groups, total Aβ accumulation did not differ significantly. It is also noted that the Aβ42: Aβ40 ratio did not differ significantly among the hiN culture groups (FIG. 5I). This contrasts with the phenotype of familial AD mutant hiN cells that display an increased Aβ42: Aβ40 ratio (18) as a consequence of altered γ-secretase activity associated with Presenilin-1 or -2 mutation (FIG. 1H). Further analysis of the hiN cultures showed that APP holoprotein levels are unaltered in level among the 3 groups, as assessed by ELISA, by quantitative analysis of immunocytochemistry (ICC) within the hiNs, as well as by Western blotting (FIG. 1J, FIGS. 5J, L, M). Aβ42 and Aβ40 fragments appeared highly stable in hiN cultures over the 48 hr time course of the analysis (as quantified in the context of the γ-Secretase inhibitor N-[(3,5-Difluorophenyl)acetyl]-L-al_anyl-2-phenyl]glycine-1,1-dimethylethyl ester; DAPT). One explanation for an increase in total Aβ, in the absence of increased APP holoprotein or altered Aβ42: Aβ40 ratio, is an increase in β-secretase activity towards APP. Consistent with this, it was found that the level of the extracellular product of the β-secretase cleavage of APP, sAPPβ, was increased in E3/4 hiN culture media (FIG. 1K). Such an increase was not apparent in the parental fibroblast cultures, and thus, without being bound by theory, cannot be due to the contaminating fibroblasts in the hiN cultures. Levels of the β-site APP cleaving enzyme 1 (BACE1) protein, as determined by either quantitative ICC or Western blotting, did not appear to differ significantly among hiN cell cultures (FIGS. 5K, L, N).

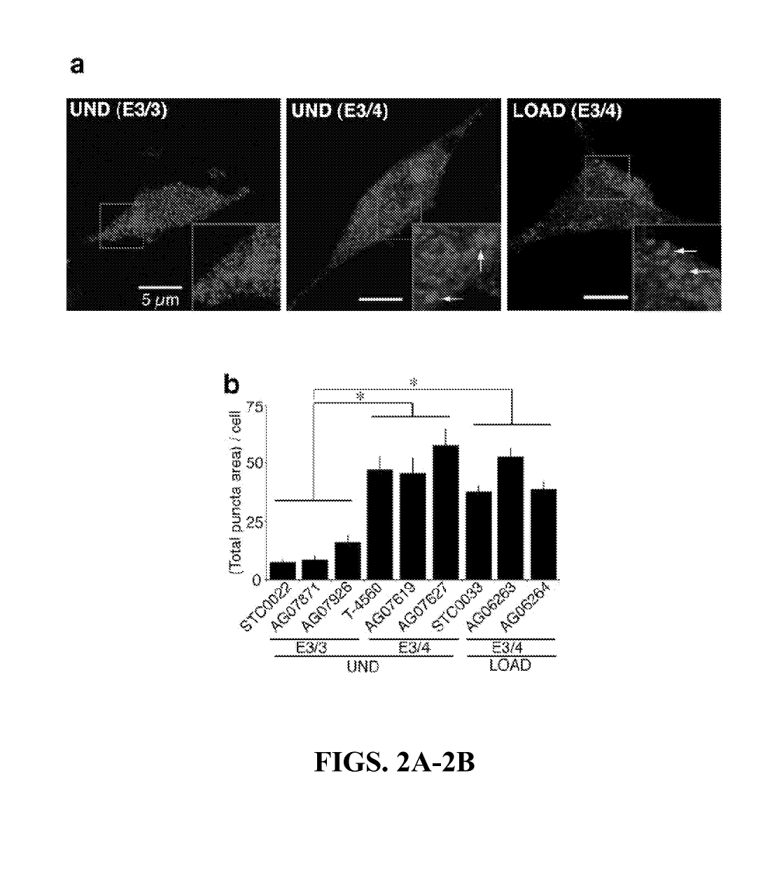

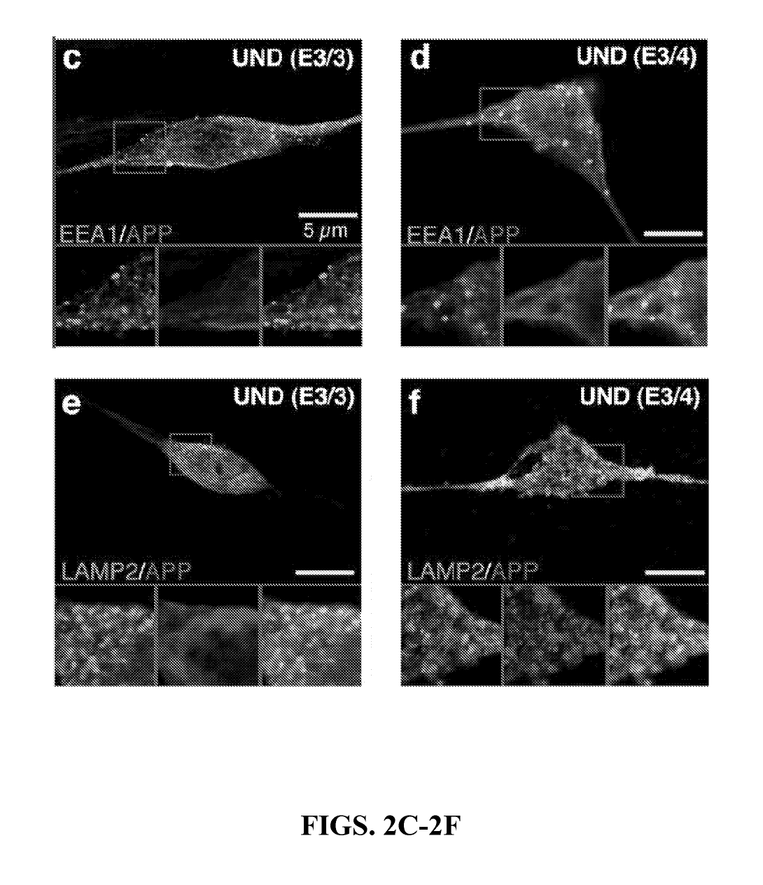

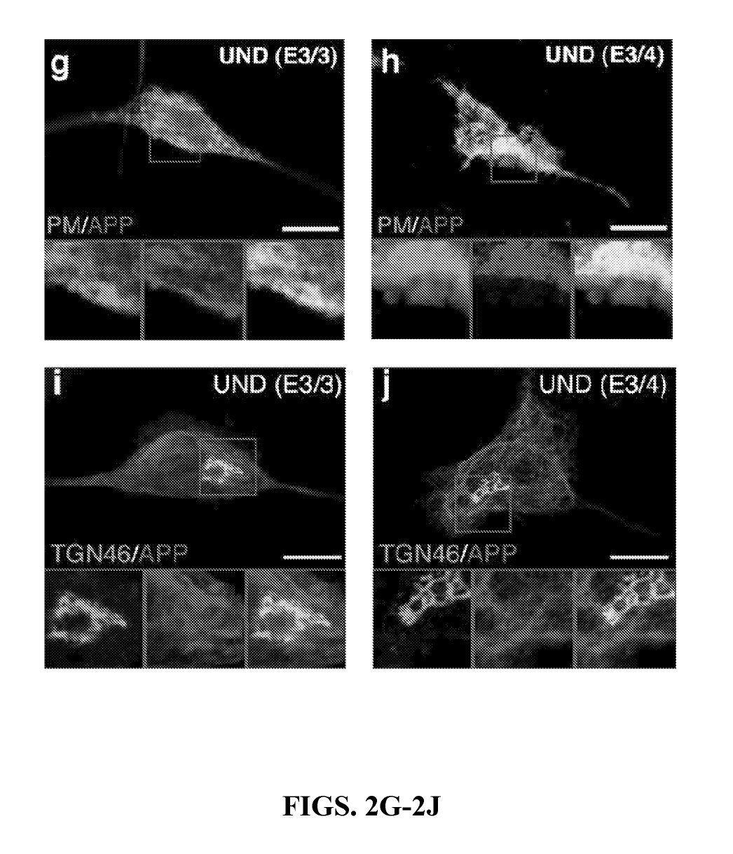

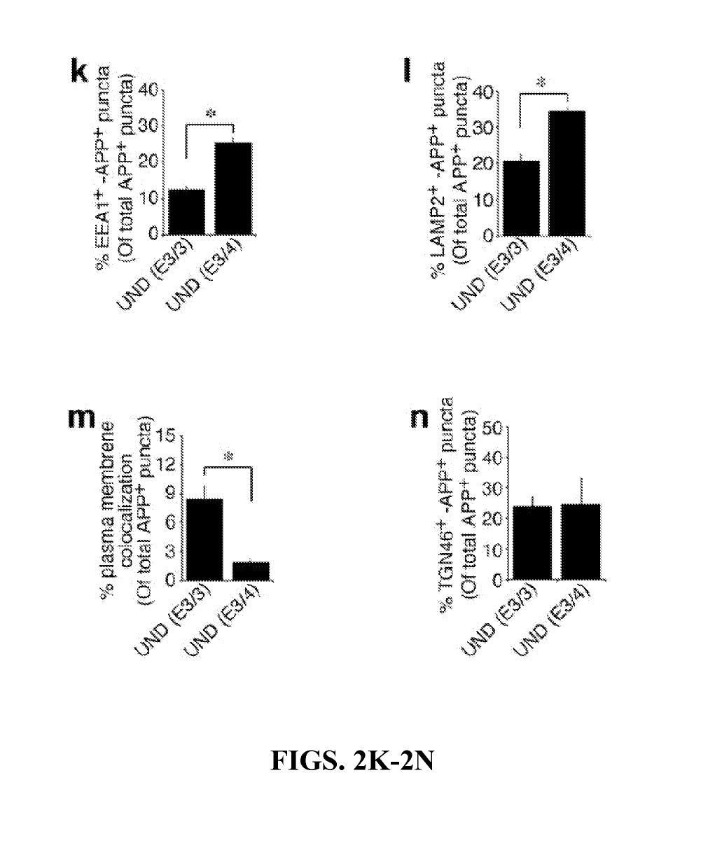

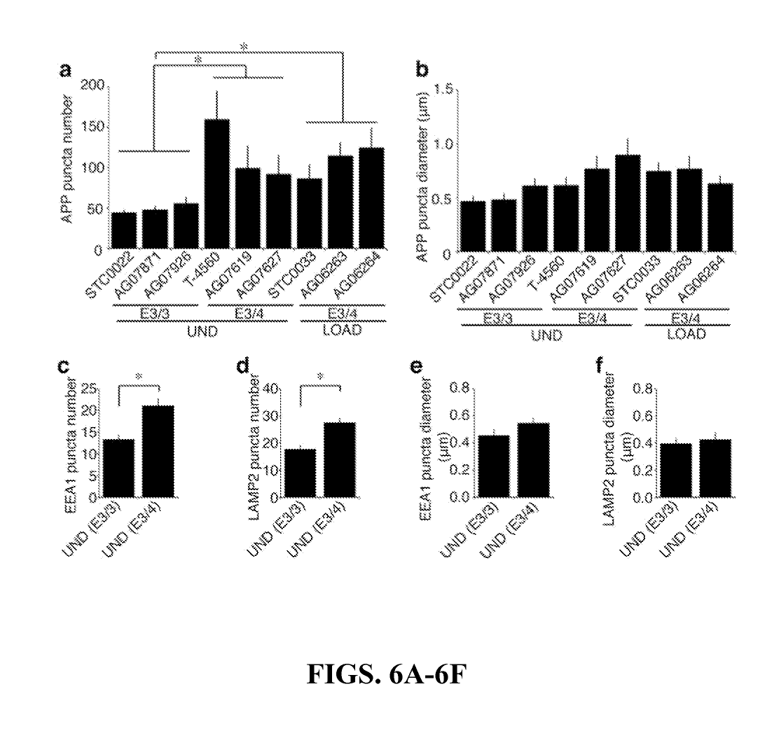

Without being bound by theory, altered processing of APP in E3/4 hiNs can be a consequence of modified subcellular localization. ICC analysis of APP localization (with a monoclonal antibody, 22C11, that recognizes the amino-terminal domain) revealed a significant increase in intracellular APP-positive puncta, both in terms of their number and total area, within E3/4 hiNs relative to the E3/3 hiNs (FIGS. 2A, B; FIGS. 6A, B). Subsets of APP-positive puncta stained positively for the early endosomal marker-1 (EEA1) or the lysosomal/late endosome marker lysosomal associated membrane protein-2 (LAMP2), as quantified by immunofluorescent confocal microscopy (FIGS. 2C-F, K, L). Furthermore, APP-positive puncta staining for either the EEA1 or LAMP2 marker was significantly increased (as a fraction of total puncta stained for APP) in E3/4 hiNs relative to the E3/3 hiNs. In contrast, cell surface localization of APP, as determined by double staining with CellMask reagent dye and the APP antibody, was significantly decreased in the E3/4 hiNs relative to E3/3 hiNs (FIGS. 2G, H, M). APP colocalization with the trans-Golgi network marker TGN46 was not altered in the E3/4 hiNs relative to E3/3 cells (FIGS. 2I, J, N). Importantly, it is noted that EEA1 and LAMP2 puncta were generally increased in number and total area (FIGS. 2R, S; FIGS. 6C-F) irrespective of APP staining. This points to a broad alteration in the endosomal compartment rather than an APP-specific defect. Co-staining of APP-positive puncta with an antibody to BACE1 (18) was increased in E3/4 hiNs (relative to E3/3 hiNs; FIGS. 2O, P, Q), consistent with the previously described enrichment of BACE1 at early and late endosomes (22, 23). As BACE1 is most active at such acidic intracellular compartments, without being bound by theory, this provides a mechanism for the increased processing of APP by BACE1 in the E3/4 hiNs.

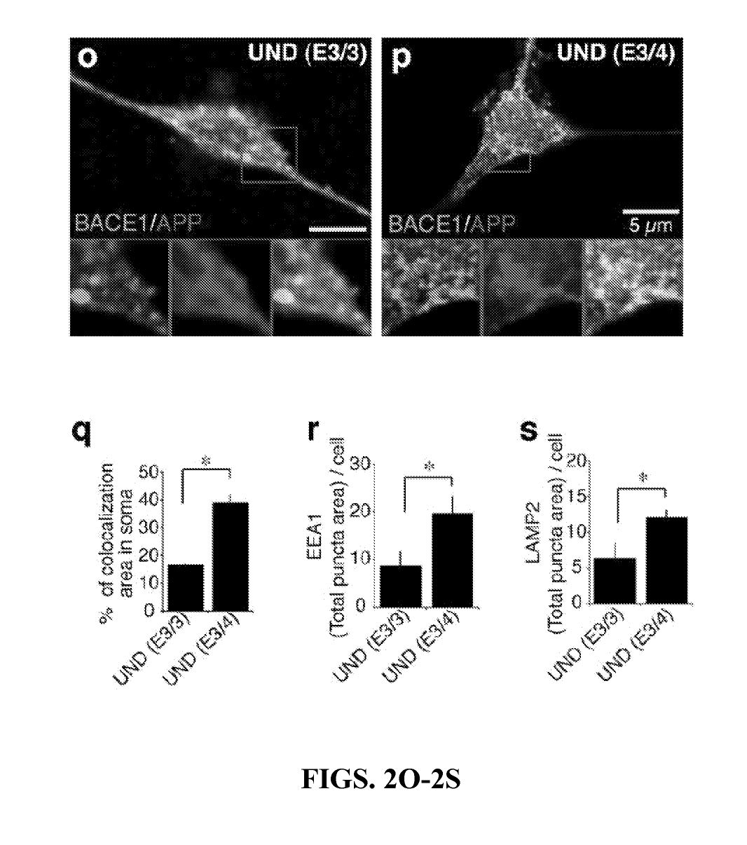

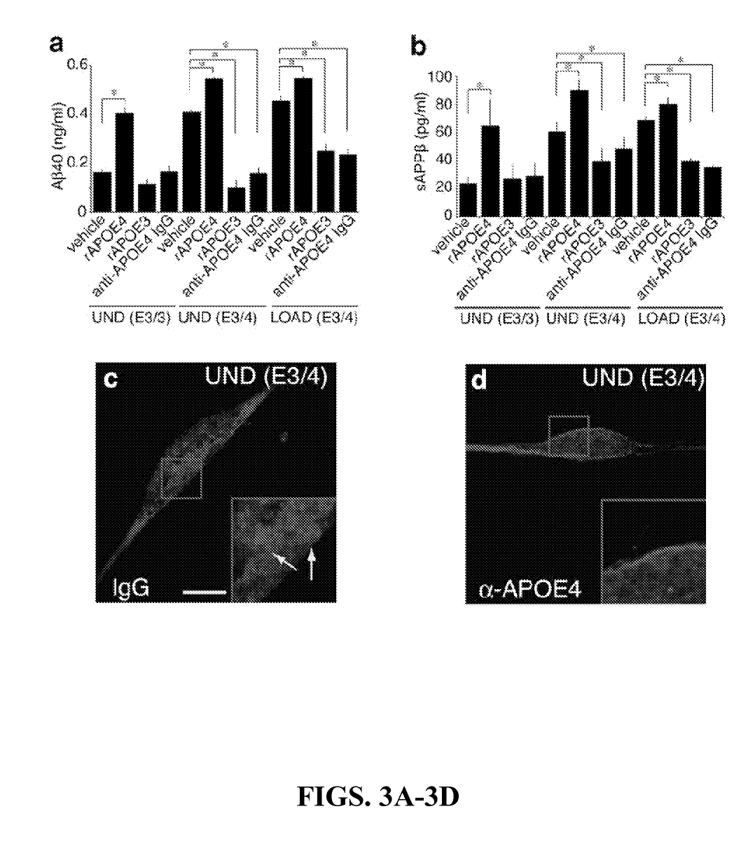

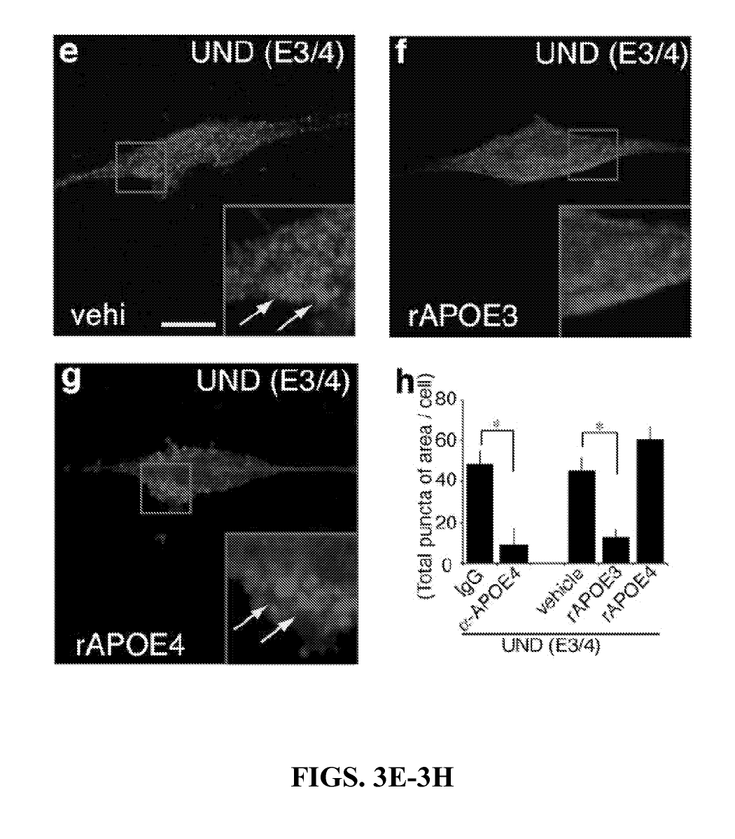

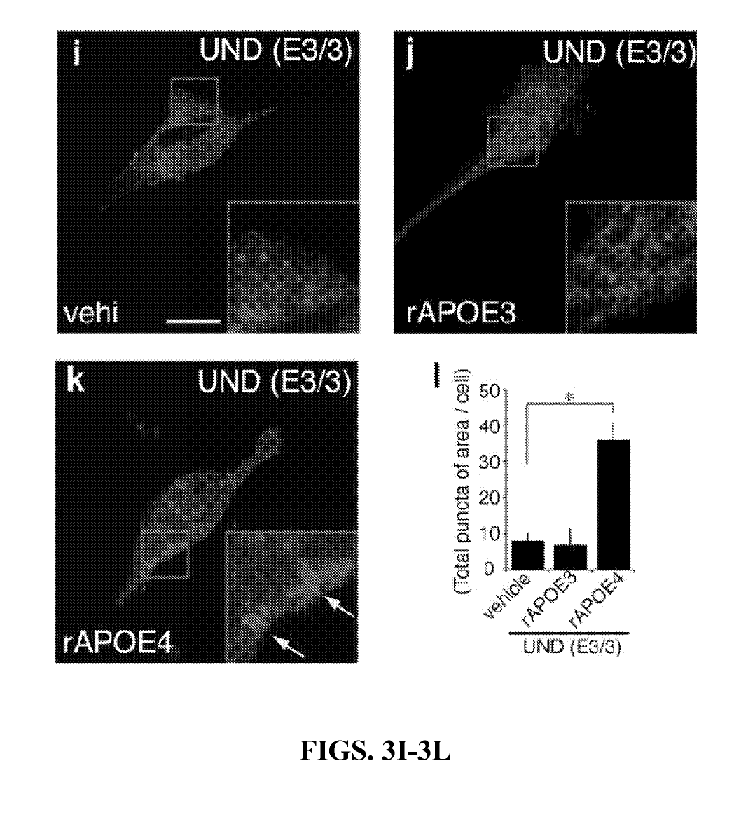

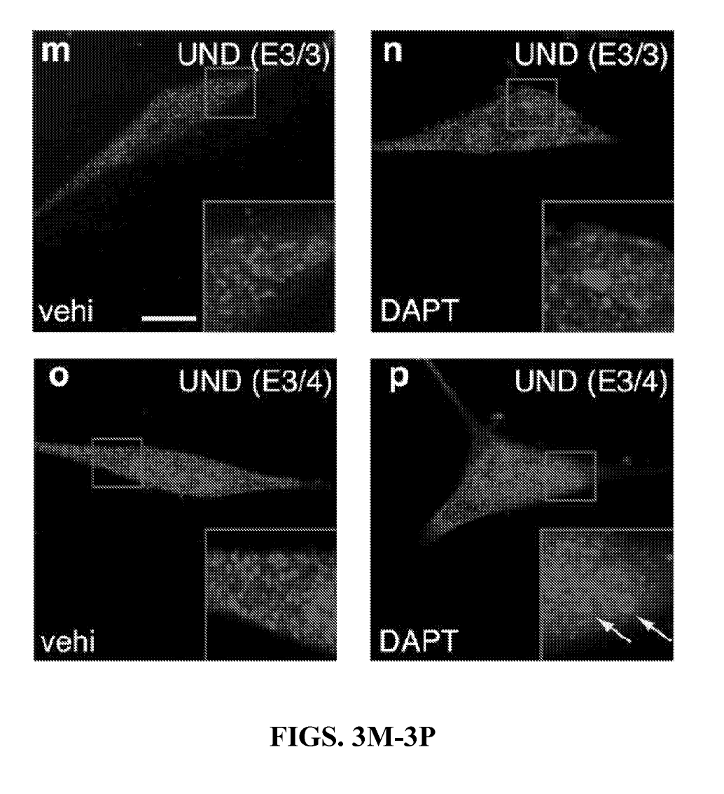

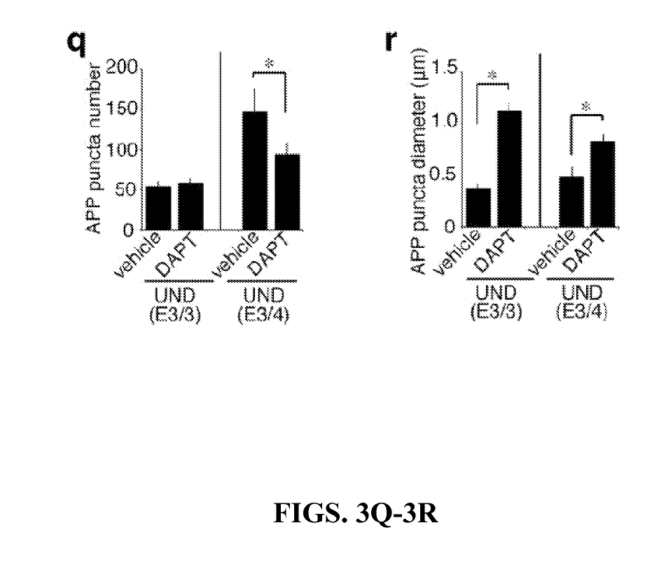

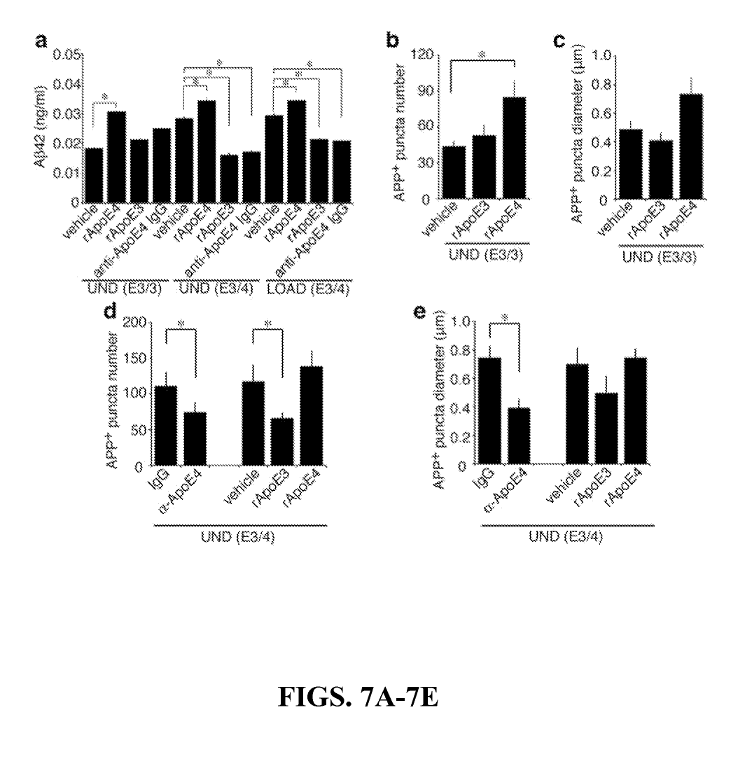

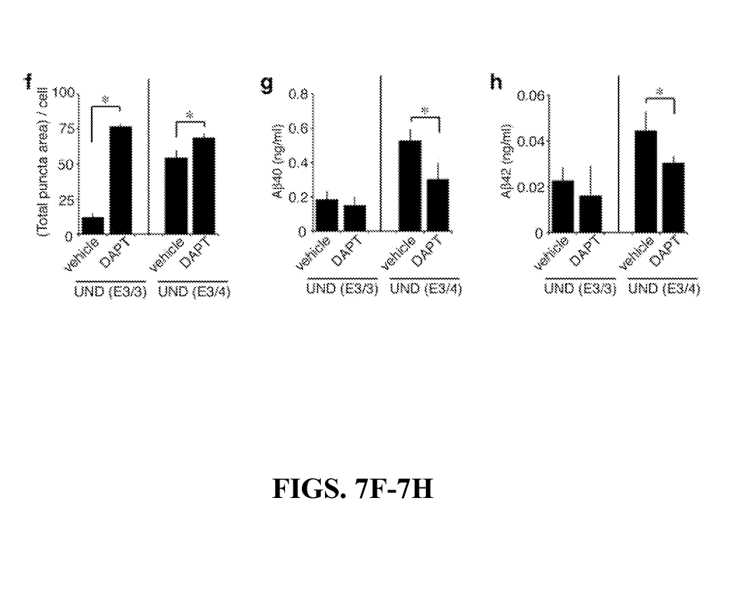

Given the genetic diversity of the human population, without being bound by theory, the phenotypes observed across the small panel of hiNs examined can correspond to other genetic or epigenetic alterations, and not to APOE variation. To address specifically the role of APOE, biochemical complementation ‘rescue’ studies were performed (FIGS. 3A-L). First, addition of a blocking antibody specific to APOE4 (but not control IgG) suppressed both the increased Aβ accumulation in the culture media of E3/4 cells as well as the alteration in intracellular APP-positive puncta (FIGS. 3A, C, D, H; FIGS. 7A, D, E). Second, addition of exogenous recombinant APOE3 protein (100 μg/ml for 48 hr) to E3/4 hiN cultures led to a significant reduction in Aβ accumulation in the culture media as well as decreased intracellular APP total puncta area (FIGS. 3A, C-H, FIGS. 7A, D, E). Third, E3/3 hiN cells cultured in the presence of exogenous recombinant APOE4 protein (100 μg/ml for 48 hr; FIG. 3A, FIG. 7A), but not exogenous APOE3 protein, showed increased total Aβ accumulation in the culture media, as previously described (13, 15). Furthermore, APOE4 treatment induced an increase in intracellular total APP puncta area in the E3/3 cells (FIGS. 3I-L, FIGS. 7B, C). In all cases Aβ level changes were paralleled by alteration in the accumulation of sAPPβ (FIG. 3B).

The above studies validate the role of APOE4 in the observed APP processing and localization phenotypes (23). Furthermore, they indicate a model whereby APOE4 leads to increased co-localization of APP with BACE1, the rate-limiting enzyme in Aβ production, and thus modified APP processing. The exclusion of an alternative model, that accumulation of Aβ in E3/4 hiNs leads secondarily to endosomal compartment alterations, was next sought. Inhibition of Aβ production using the γ-secretase inhibitor DAPT failed to suppress the endosomal phenotype of E3/4 hiNs (FIGS. 3M-R, FIG. 7F), rendering the latter model unlikely. Rather, DAPT treatment led to a selective increase in the size of individual puncta, and thus an increase in total APP puncta area in E3/3 cells, similar to the phenotype of familial AD PSEN mutant hiNs (18). The common aspect of the cellular phenotype observed in familial (18) AD and LOAD/APOE4 hiN models—increased total area of APP-positive endosomal puncta—indicates a shared disease mechanism.

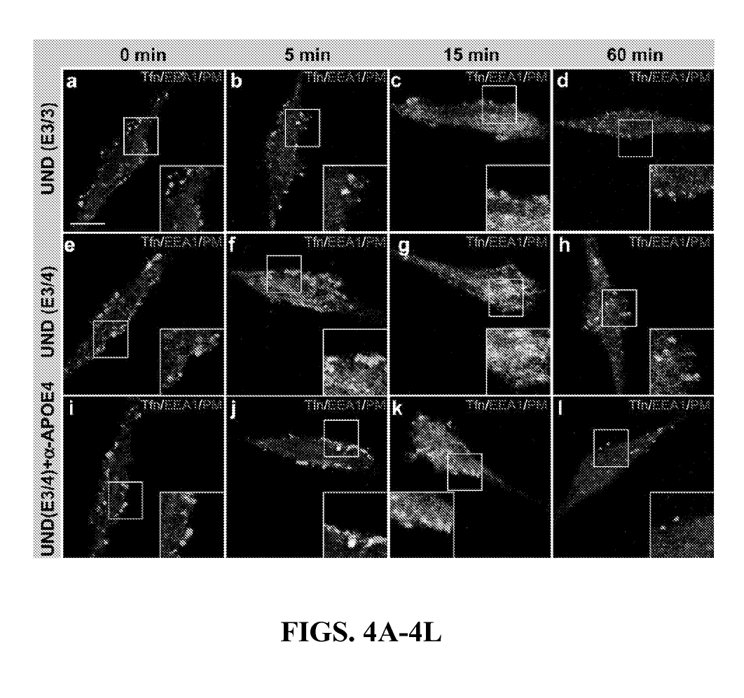

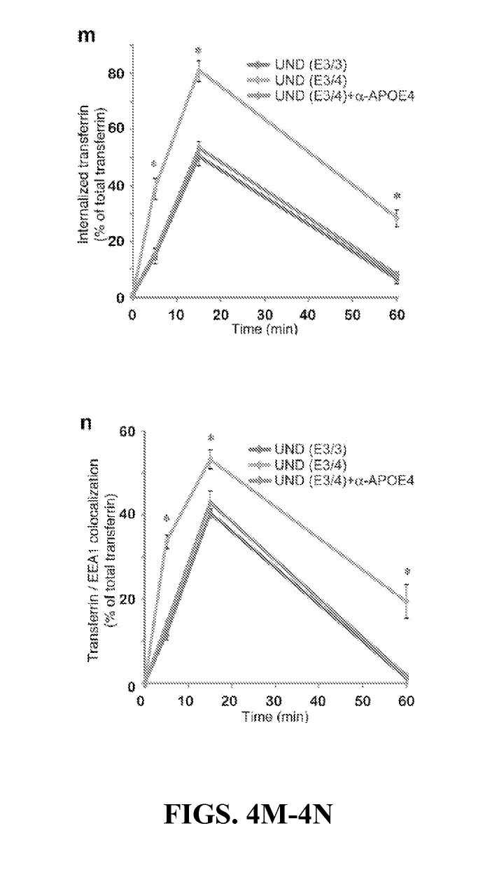

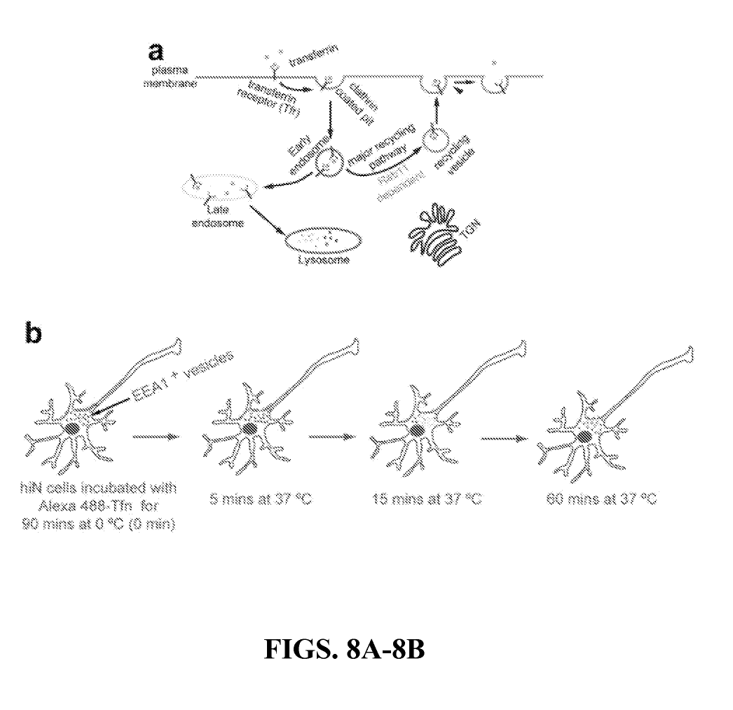

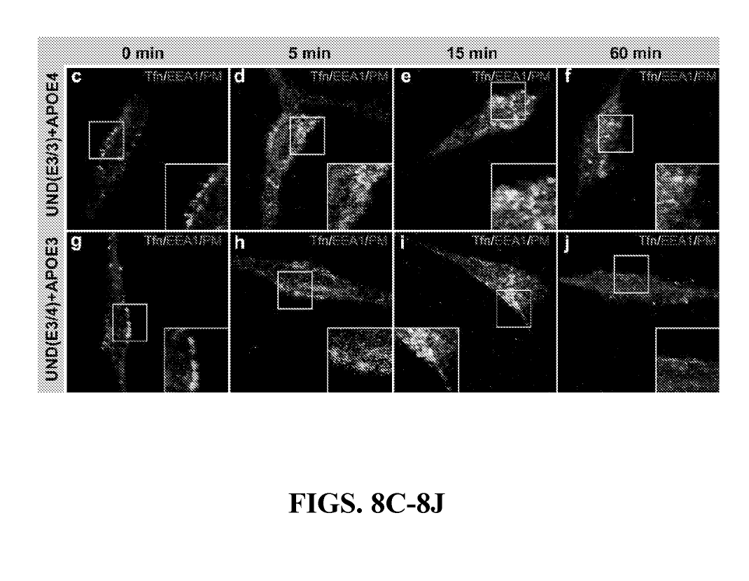

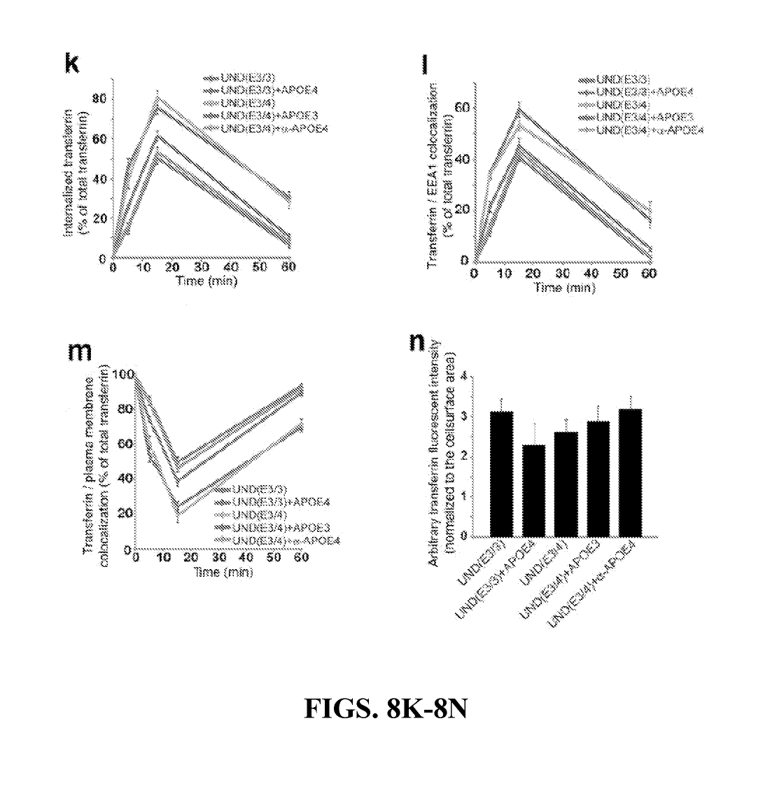

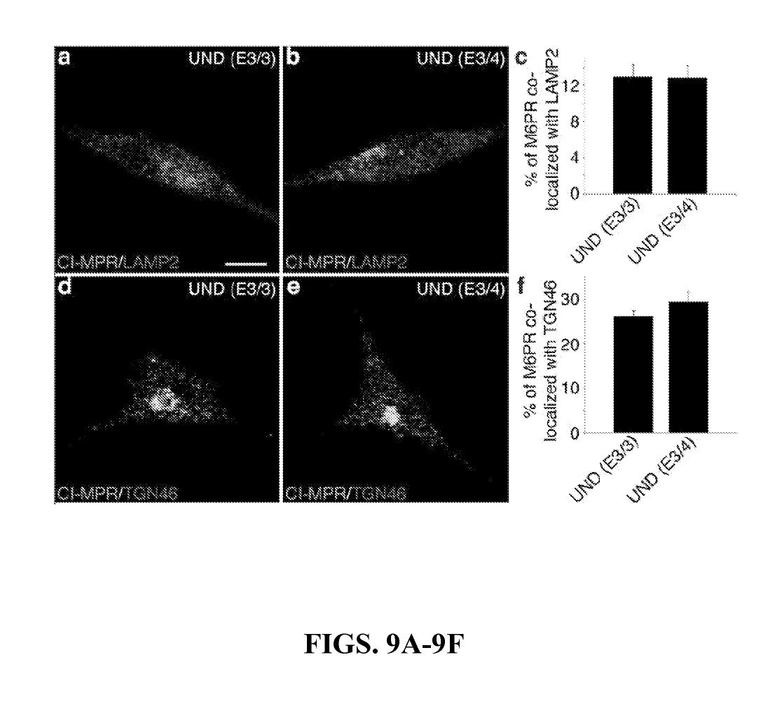

As increased intracellular puncta area in E3/4 hiNs was apparent not only with APP staining, but even upon staining for the general early endosome marker EEA1 (FIG. 2R), this indicated a broad modification in vesicular endocytic trafficking. General vesicular internalization, trafficking, and recycling within the early endocytic compartment was queried with transferrin (Tfn), a commonly used marker protein that binds to its cell surface receptor leading to clathrin-mediated endocytosis (24) (FIG. 8A). E3/3 (AG07926 culture with or without added APOE4 protein) or E3/4 (T-4560 culture with or without added APOE3 protein or anti-APOE4 antibody) hiN cultures were incubated with fluorescent Alexa 488-tagged transferrin (Alexa-Tfn) on ice at 0° C. for 90 min and then either fixed directly, or allowed 5 to 60 minutes of internalization at 37° C. prior to fixation. Subsequently, cells were immunostained for the early endosome marker EEA1 as well as stained with the CellMask membrane dye. Confocal fluorescent microscopy demonstrated significantly accelerated internalization of Alexa-Tfn from the cell surface in E3/4 hiNs relative to E3/3 hiNs (FIGS. 4A-H), with an accelerated increase in staining at EEA1-positive early endosome puncta, and a corresponding rapid reduction in co-labeling of Alexa-Tfn with CellMask at the cell surface (FIGS. 4M, N, FIGS. 8K-M). Initial Tfn receptor binding did not appear different among the genotypes during incubation at 0° C. (FIGS. 4A, E, I, M, N; FIG. 8C, G, K-N). At late time points, the E3/4 group demonstrated consistently increased retained internalized Tfn at the early endosome structures relative to the E3/3 group (FIGS. 4B-D, F-H, M, N). Addition of anti-APOE4 antibody or APOE3 protein to E3/4 cells suppressed the altered Tfn internalization phenotypes (FIGS. 4J-L, M, N, FIGS. 8G-J, K, L). In contrast, addition of APOE4 protein to E3/3 hiNs led to a phenocopy of the E3/4 endocytosis alterations. To further probe the integrity of endocytic trafficking in E3/4 hiNs, sorting of the cation-independent mannose-6-phosphate receptor (CI-MPR), a commonly used marker that is typically transported from early endosomes either to the trans-Golgi network (TGN), as mediated by the retromer complex, or to late endosomes and lysosomes for degradation (25), was also evaluated. CI-MPR localization appeared unaltered in the E3/4 hiNs (relative to E3/3 hiNs; FIG. 9), arguing against a general abrogation of vesicular trafficking downstream of receptor-mediated endocytosis from the plasma membrane to early endosomes.

The altered APP processing and endocytic trafficking defects seen in E3/4 hiNs are reminiscent of clinical LOAD pathology. Pathological analysis by Cataldo et al. previously described altered early endosomal vesicular compartments in LOAD patient brain (26, 27, 28). As the E3/4 phenotypes are apparent even when cells are obtained from apparently unaffected individual, these data indicate a pre-disease state is present in neurons of individuals that harbor an APOE4 allele. Indeed, non-demented carriers of the APOE4 allele display altered cognitive function (29) and neuroimaging findings at brain regions afflicted in LOAD such as the entrorhinal (30).

Prior in vivo and in vitro studies have led to conflicting models for the mechanism of action of APOE4 in LOAD (9, 10, 13, 14, 15). In vivo isotope labeling studies have pointed to a role for APOE4 in altered clearance of Aβ from the CNS (9, 10, 12). Other studies have indicated a more direct mode of action for APOE4 in neuronal APP generation (13, 14, 15). The data herein are consistent with a model whereby APOE4 prolongs the transit time of APP in early endosomes through accelerated internalization and reduced removal from this compartment. Without being bound by theory, APOE4 binding to receptors such as LRP (31) or APOER2 (15) on neurons, inducing receptor-mediated endocytosis of such receptors, can promote concomitant APP internalization through adaptor proteins such as X11 (15). Alternatively, APOE4 can function more indirectly at neurons, by modifying lipid signaling or trafficking within these neurons. The roles of APOE4 on production and clearance of Aβ are not mutually exclusive. Finally, it is noted that the APOE4 pathway is a particularly attractive drug target for LOAD. Compounds that suppress the cellular impact of APOE4 on hiN cells can serve as useful therapeutics for LOAD.

Methods

Human skin fibroblast cultures from 9 individuals were obtained as de-identified, banked tissue samples. Cell culture, hiN conversion, FACS analyses, qRT-PCR, APP processing, and immunohistological analyses were performed as detailed in Qiang et al. (18). For APOE protein, recombinant human APOE3 and APOE4 were purchased from Biovision Inc., and anti-apolipoprotein E4 mouse IgG (1F9) was from MBL. These were added to hiN cell cultures for 48 hrs prior to the analyses. Transferrin uptake and recycling assays are detailed below. The statistical significance of all comparisons was assessed by non-parametric ANOVA Kruskal-Wallis H-test followed, as indicated, followed by post-hoc Mann-Whitney U-test, with Bonferroni correction.

Human Skin Fibroblasts.

Human skin fibroblast cultures from 9 individuals were used in this study (see Table 1). All of these were de-identified, banked tissue samples. There was no interaction with subjects, no intervention, and private, identifiable information was not collected. 3 fibroblast lines were obtained from unaffected individuals which have E3/3 allele (STC0022 [female, 65yo]; AG07871 [female, 49yo]; AG07926 [female, spouse of an AD patient, precise age data not available]); 3 from unaffected individuals which have E3/4 allele (T-4560 [male, 89+yo]; AG07619 [male, 68yo]; and AG07627 [female, 49yo); as well as 3 late-onset Alzheimer's disease (LOAD) lines (AG06264 [female, 62yo], AG06263 [female, 67yo] and STC0033 [male, 81yo]). Unidentifiable, anonymous samples, including STC0022 and STC0033; T-4560 were obtained. Details for all other cultures are available at http://ccr.coriell.org/. Diagnosis is based on clinical diagnosis and pathological diagnosis. Human skin fibroblasts were cultured in standard fibroblast media (DMEM with 10% FBS).

hiN Cell Induction and Transfection.

Fibroblasts were plated at 20,000 cells/well in 24-well plates one day before infection. Culture plates and dishes were treated with Poly-L-Ornithine (Sigma) and Laminin (Invitrogen) or Poly-D lysine (Trevigen) and Laminin before the application of the cells as per the manufacturer's instructions. Fibroblasts were transduced with replication-incompetent, VSVg-coated lentiviral particles encoding Ascl1, Brn2, Zic 1 and Mytl1, in fibroblast media containing polybrene (8 μg/ml). Each lentiviral type was added at a multiplicity of infection ˜2:1. Two day after transduction, the media was replaced with glial-conditioned N2 media (1) containing 20 ng/ml BDNF (Peprotech). For the first 4-6 days in N2 media, dorsomorphin (1 μM; Stemgent) was applied to the culture. Media was changed every 2-3 days for the duration of the culture period. In certain cases, hiN cells co-cultured with rat astrocytes (detailed methods see Qiang et al., 2011) (1).

Immunocytochemistry.

Cells were fixed with 4% paraformaldehyde for 10 min at room temperature, followed by rinsing 3 times with phosphate-buffered saline (PBS). Cells were then permeabilized with 0.1% Triton X-100 in 1XPBS for 10 min at room temperature. After rinsing three times again with PBS, cells were incubated with blocking buffer containing 10% goat serum and 10 mg/ml BSA at room temperature for 1 hr. All primary antibodies were diluted in 1XPBS solutions. Cells were incubated with primary antibodies as listed at 4° C. for 12-16 hours, followed by the corresponding secondary antibody solutions in 37° C. for 1 hr. Cells were rinsed with 1XPBS three times followed by mounting coverslips with anti-fade solution (Invitrogen). Primary antibodies are: mouse anti Tuj1 (Covance, 1:1000); rabbit anti Tuj1 (Covance, 1:2000); rabbit anti MAP2 (Sigma, 1:400); mouse anti MAP2 (Sigma, 1:500); mouse anti tau (tau1, Millipore, 1:500); mouse anti NeuN (Millipore, 1:200); rabbit anti vGLUT1 (Synaptic System, 1:100); and chicken anti Tbr1 (Millipore, 1:500). Also used were: mouse anti APP (22C11, Millipore, 1:500), rabbit anti APP (KDI, Millipore, 1:500), rabbit anti BACE1 (invitrogen, 1:500, sheep anti TGN46 (AbDserotec, 1:500), mouse anti TGN46 (Abcam, 1:500), rabbit anti EEA1 (Millipore, 1:500), mouse anti CI-MPR (Abcam, 1:500), rabbit anti LAMP2 (Sigma, 1:400). Dylight 488-, Dylight 549- and Dylight 649-conjugated secondary antibodies were purchased from Jackson Immunoresearch. Alexa-488, Alexa-633-conjugated secondary antibodies were obtained from Invitrogen. Subsequently, pictures were analyzed for cell quantification and fluorescent intensity using Image J 1.42q (National Institute of Health, USA) software. For each quantification, values are given as mean±SEM. Immunocytochemistry was analyzed by laser-scanning confocal microscopes (LSM510, Carl Zeiss, Göttingen, Germany) with a 63×/1.4 objective or Olympus 1X71 epifluorescent microscope (Olympus, Japan) with 10×, 20× and 40× objectives. hiN cell counts and fluorescence intensity analysis were performed by taking 10 to 35 images of randomly selected views per coverslip. Fluorescence was quantified by means of confocal microscopy using a 63× objective. Subsequently, images were analysed for cell quantification and fluorescent intensity using Image J 1.42q (National Institute of Health, USA) software.

FACS and qPCR.

Detailed methods are as previously described (1). Briefly, hiN cells (10⁶ cells) were stained with an antibody to NCAM (BD Bioscience) and then sorted on a FACS Aria IIu (BD Bioscience, CA) directly into RNA lysis solution (Ambion, Tex.). RNA was extracted from cell preparations using the RNAqueous Micro Kit (Ambion). Concentration and quality of RNA were assessed using the Bioanalyzer system (Agilent). Quantitative real time RT-PCR was carried out as described (Qiang et al., 2011); primer pairs utilized are listed below. Gene expression levels were quantified by the ΔΔCt method (1). Primer pairs: for NCAM (Fwd: GGA TCT CAG TGG TGT GGA ATG, Rev: TGG CGT TAT AGA TGG TGA GGG); for microtubule-associated protein 2 (MAP2, Fwd: CTC AGC ACC GCT AAC AGA GG, Rev: CTC CGC CTT GAT CCT TAA TCT C); for Gamma-aminobutyric acid (GABA) B receptor 2 (GABBR2) (Fwd: CCG CAA CGA GTC ACT CCT G, Rev: CAC TCC GTG TCA TAG AGC CG); for 5-hydroxytryptamine (serotonin) receptor 2A (HTR2A, Fwd: CTT TGT GCA GTC TGG ATT TAC CT, Rev: ACT GAT ATG GTC CAA ACA GCA AT); for synaptosomal-associated protein, 25 kDa (SNAP25, Fwd: ACC AGT TG GCT GAT GAG TCG, Rev: TCT TCA ACC AGT TGC AGC ATA C).

Sandwich ELISAs.

APP ELISA and APOE4/pan-APOE ELISA were performed using each a human APP ELISA kit (Invitrogen, Camarillo, Calif.) and APOE4/pan-APOE ELISA kit (MBL, Eoburn, Mass.), according to the manufacturer's instruction. Absorbance was read on a Microplate Reader (Infinite® M200, TECAN, Männedorf Switzerland) at 450 nm. The amount of APP was normalized to the total cell protein (determined with the DC Protein Assay Reagent kit; Bio-Rad, Hercules, Calif.). sAPPβ and Aβ ELISA were performed on supernatant media from hiN cell cultures at 21 days after viral transduction. sAPPβ ELISA was performed using BetaMark™ sAPP Beta ELISA kit (Covance, Princeton, N.J.), according to the manufacturer's instruction. The chemiluminescence was read on a microplate luminometer (Infinite® M200). Aβ quantification was performed by ELISA as described previously (2). Media was conditioned for 48 hr prior to harvesting. Samples were analyzed for Aβ40 or Aβ42 using specific sandwich ELISAs. Briefly, Aβ40, and Aβ42 were captured using monoclonal antibodies targeted against amino acids 35-40 (HJ2.0), or 33-42 (HJ7.4) of Aβ, respectively. For Aβ40 and Aβ42 assays, a biotinylated central domain monoclonal antibody (HJ5.1) followed by streptavidin-poly-HRP-40 was used for detection (Sigma). All assays were developed using Super Slow ELISA TMB (Sigma) and read on a Microplate Reader (Infinite® M200) at 650 nm. ELISA signals were reported as the mean±SEM of three replica wells in ng of Aβ per ml supernatant, based on standard curves using synthetic Aβ40 and Aβ42 peptides (rPeptide; Bogart, Ga.). Samples was optimized to detect Aβ40 and Aβ42 in the ranges of 1-3,000 ng/ml and 0.03-30 ng/ml, respectively (1). The amount of sAPPβ and Aβ was normalized to the cell number per well as indicated.

Immunoblotting.

HiNs were suspended in RIPA buffer contained protease inhibitor cocktail (Sigam) and sonicated, following cells were incubated for 1 hr at 4° C. The lysates were cleared by centrifugation at 10,000×g for 10 minutes at 4° C. Protein concentration was determined by Bio-Rad Dc Protein assay kits (Bio-Rad), and 5 μg of total protein lysate was resolved on a 4-12% SDS-PAGE gel. Protein samples were transferred onto nitrocellulose membranes using the semi-dry transfer unit (Owl scientific, Woburn, Mass.) and blocked with 3% skim milk in Tris buffered saline with 0.1% tween 20 for 1 hour. And treatment of primary antibodies were incubated overnight at 4° C., and primary antibody binding was detected using horseradish peroxides conjugated anti-mouse IgG at 1:5,000 dilution (Jackson immunolaboratories) or horseradish peroxides conjugated anti-rabbit IgG at 1:5,000 dilution (Jackson immunolaboratories).

APOE Treatment.

Recombinant human APOE3 and APOE4 were purchased from Biovision Inc. (Milpitas, Calif.), and anti-apolipoprotein E4 mouse IgG (1F9) was from MBL. To determine Aβs and sAPPβ in cultured supernatant, these APOEs (100 μg/ml) or anti-APOE4 IgG (2 μg/ml) were added to hiN cell cultures incubated for 2 weeks after infection, and these media were collected after 48 hr treatment.

Transferrin Uptake and Recycling Assay.

hiN cultures were maintained on glass coverslips in 24 well plates for 2 to 3 weeks before the assays. Prior to the assay, media was replaced with ice-cold N2 medium containing Alexa-488 tranferrin (Invitrogen, CA) with the final concentration at 50 ug/ml. Cultures were then incubated on ice (0° C.) for 90 minutes, followed by 3 rinses in ice cold culture medium to remove unbound ligand. Some cultures were fixed with 4% paraformaldehyde in PBS at at 4° C. this point in time where indicated. For other cultures, ice-cold culture medium was replaced with 37° C. fresh culture medium with 5% FBS and incubated at 37° C. for various lengths of time as indicated. At each time point, cells were washed with ice-cold PBS and fixed immediately at 4° C. as above. For the rescue experiments, recombinant APOE3 or APOE4, or anti-ApoE4 antibody, were added into the cultures as described above 48 hr prior to the assays. ICC analyses were carried out with organelle markers as indicated.

Statistical Analysis.

Statistic analyses were performed with the Ystat 2002 software (Igaku Tosho Shuppan Co., Ltd., Tokyo, Japan) together with Microsoft Excel software (Microsoft Corp., Redmond, Wash., USA). The statistical significance of all comparisons was assessed by non-parametric ANOVA Kruskal-Wallis H-test followed, as indicated, followed by posthoc_Mann-Whitney U-test, with Bonferroni correction.

### REFERENCES

1. Hardy, J. & Selkoe, D. J. The amyloid hypothesis of Alzheimer's disease: progress and problems on the road to therapeutics. Science (New York, N.Y. 297, 353-356 (2002).
2. Harold, D. et al. Genome-wide association study identifies variants at CLU and PICALM associated with Alzheimer's disease. Nat Genet 41, 1088-1093 (2009).
3. Hollingworth, P. et al. Common variants at ABCA7, MS4A6A/MS4A4E, EPHA1, CD33 and CD2AP are associated with Alzheimer's disease. Nat Genet 43, 429-435 (2011).
4. Lambert, J. C. et al. Genome-wide association study identifies variants at CLU and CR1 associated with Alzheimer's disease. Nat Genet 41, 1094-1099 (2009).
5. Naj, A. C. et al. Common variants at MS4A4/MS4A6E, CD2Aβ, CD33 and EPHA1 are associated with late-onset Alzheimer's disease. Nat Genet 43, 436-441 (2011).
6. van Es, M. A. & van den Berg, L. H. Alzheimer's disease beyond APOE. Nat Genet 41, 1047-1048 (2009).
7. Strittmatter, W. J. et al. Apolipoprotein E: high-avidity binding to beta-amyloid and increased frequency of type 4 allele in late-onset familial Alzheimer disease. Proc Natl Acad Sci USA 90, 1977-1981 (1993).
8. Genin, E. et al. APOE and Alzheimer disease: a major gene with semi-dominant inheritance. Mol Psychiatry 16, 903-907 (2011).
9. Huang, Y. Abeta-independent roles of apolipoprotein E4 in the pathogenesis of Alzheimer's disease. Trends Mol Med 16, 287-294 (2010).
10. Kim, J., Basak, J. M. & Holtzman, D. M. The role of apolipoprotein E in Alzheimer's disease. Neuron 63, 287-303 (2009).
11. Mahley, R. W. & Rall, S. C., Jr. Apolipoprotein E: far more than a lipid transport protein. Annu Rev Genomics Hum Genet 1, 507-537 (2000).
12. Castellano, J. M. et al. Human apoE isoforms differentially regulate brain amyloid-beta peptide clearance. Sci Transl Med 3, 89ra57 (2011).
13. Ye, S. et al. Apolipoprotein (apo) E4 enhances amyloid beta peptide production in cultured neuronal cells: apoE structure as a potential therapeutic target. Proc Natl Acad Sci USA 102, 18700-18705 (2005).
14. Dodart, J. C. et al. Apolipoprotein E alters the processing of the beta-amyloid precursor protein in APP(V717F) transgenic mice. Brain Res 955, 191-199 (2002).
15. He, X. et al. Apolipoprotein receptor 2 and X11 alpha/beta mediate apolipoprotein E-induced endocytosis of amyloid-beta precursor protein and beta-secretase, leading to amyloid-beta production. J Neurosci 27, 4052-4060 (2007).
16. Huang, Y. Molecular and cellular mechanisms of apolipoprotein E4 neurotoxicity and potential therapeutic strategies. Curr Opin Drug Discov Devel 9, 627-641 (2006).
17. Chen, Y., Durakoglugil, M. S., Xian, X. & Herz, J. ApoE4 reduces glutamate receptor function and synaptic plasticity by selectively impairing ApoE receptor recycling. Proc Natl Acad Sci USA 107, 12011-12016 (2010).
18. Qiang, L. et al. Directed conversion of Alzheimer's disease patient skin fibroblasts into functional neurons. Cell 146, 359-371 (2011).
19. Pang, Z. P. et al. Induction of human neuronal cells by defined transcription factors. Nature 476, 220-223 (2011).
20. Vierbuchen, T. et al. Direct conversion of fibroblasts to functional neurons by defined factors. Nature 463, 1035-1041 (2010).
21. Yoo, A. S. et al. MicroRNA-mediated conversion of human fibroblasts to neurons. Nature 476, 228-231 (2011).
22. Small, S. A. & Gandy, S. Sorting through the cell biology of Alzheimer's disease: intracellular pathways to pathogenesis. Neuron 52, 15-31 (2006).
23. Vassar, R., Kovacs, D. M., Yan, R. & Wong, P. C. The beta-secretase enzyme BACE in health and Alzheimer's disease: regulation, cell biology, function, and therapeutic potential. J Neurosci 29, 12787-12794 (2009).
24. Ciechanover, A., Schwartz, A. L., Dautry-Varsat, A. & Lodish, H. F. Kinetics of internalization and recycling of transferrin and the transferrin receptor in a human hepatoma cell line. Effect of lysosomotropic agents. J Biol Chem 258, 9681-9689 (1983).
25. Seaman, M. N. Recycle your receptors with retromer. Trends Cell Biol 15, 68-75 (2005).
26. Cataldo, A. M. et al. Endocytic pathway abnormalities precede amyloid beta deposition in sporadic Alzheimer's disease and Down syndrome: differential effects of APOE genotype and presenilin mutations. Am J Pathol 157, 277-286 (2000).
27. Cataldo, A. et al. Endocytic disturbances distinguish among subtypes of Alzheimer's disease and related disorders. Ann Neurol 50, 661-665 (2001).
28. Cataldo, A. M., Barnett, J. L., Pieroni, C. & Nixon, R. A. Increased neuronal endocytosis and protease delivery to early endosomes in sporadic Alzheimer's disease: neuropathologic evidence for a mechanism of increased beta-amyloidogenesis. J Neurosci 17, 6142-6151 (1997).
29. Wolk, D. A. & Dickerson, B. C. Apolipoprotein E (APOE) genotype has dissociable effects on memory and attentional-executive network function in Alzheimer's disease. Proc Natl Acad Sci USA 107, 10256-10261 (2010).
30. Burggren, A. C. et al. Reduced cortical thickness in hippocampal subregions among cognitively normal apolipoprotein E e4 carriers. Neurolmage 41, 1177-1183 (2008).
31. Trommsdorff, M., Borg, J. P., Margolis, B. & Herz, J. Interaction of cytosolic adaptor proteins with neuronal apolipoprotein E receptors and the amyloid precursor protein. The Journal of biological chemistry 273, 33556-33560 (1998).
32. Qiang, L. et al. Directed conversion of Alzheimer's disease patient skin fibroblasts into functional neurons. Cell 146, 359-371 (2011).
33. Cirrito, J. R. et al. In vivo assessment of brain interstitial fluid with microdialysis reveals plaque-associated changes in amyloid-beta metabolism and half-life. J Neurosci 23, 8844-8853 (2003).
34. Lee J, Retamal C, Cuitino L, Caruano-Yzermans A, Shin J E, et al. Adaptor protein sorting nexin 17 regulates amyloid precursor protein trafficking and processing in the early endosomes. J Biol Chem. 2008; 283:11501-8.
35. Koo E H, Sisodia S S, Archer D R, Martin L J, Weidemann A, et al. Precursor of amyloid protein in Alzheimer disease undergoes fast anterograde axonal transport. Proc Natl Acad Sci USA. 1990; 87:1561-65.
36. Huang, 2010, Abeta-independent roles of apolipoprotein E4 in the pathogenesis of Alzheimer's disease, Trends Mol Med., 16(6):287-94. Epub 2010 May 27.
37. Kim et al., 2009, The role of apolipoprotein E in Alzheimer's disease, Neuron. 63(3):287-303.
38. Rocca, Hofman et al., 1991, Frequency and distribution of Alzheimer's disease in Europe: a collaborative study of 1980-1990 prevalence findings. The EURODEM-Prevalence Research Group, Ann Neurol. 3:381-90.
39. Friedland, 1993, Epidemiology, education, and the ecology of Alzheimer's disease. Neurology. 43(2):246-9.
40. Alzheimer, A. (1907). Uber eine eigenartige Erkrankung der Hirnrinde. Allg Z Psychiat Psych-Gerichtl Med 64, 146-148.
41. Duyckaerts, C., Delatour, B., and Potier, M. C. (2009). Classification and basic pathology of Alzheimer disease. Acta Neuropathol 118, 5-36.
42. Mucke, L. (2009). Neuroscience: Alzheimer's disease. Nature 461, 895-897.
43. De Strooper, B., and Annaert, W. (2010) Novel research horizons for presenilins and gamma-secretases in cell biology and disease. Annu Rev Cell Dev Biol 26, 235-260.
44. Parks, A. L., and Curtis, D. (2007). Presenilin diversifies its portfolio. Trends Genet 23, 140-150.
45. Pimplikar, S. W., Nixon, R. A., Robakis, N. K., Shen, J., and Tsai, L. H. (2010) Amyloid-independent mechanisms in Alzheimer's disease pathogenesis. J Neurosci 30, 14946-14954.
46. Shen, J., and Kelleher, R. J., 3rd (2007). The presenilin hypothesis of Alzheimer's disease: evidence for a loss-of-function pathogenic mechanism. Proc Natl Acad Sci USA 104, 403-409.
47. Bertram, L., Lill, C. M., and Tanzi, R. E. (2010). The genetics of Alzheimer disease: back to the future. Neuron 68, 270-281.
48. Gatz, M., Reynolds, C. A., Fratiglioni, L., Johansson, B., Mortimer, J. A., Berg, S., Fiske, A., and Pedersen, N. L. (2006). Role of genes and environments for explaining Alzheimer disease. Arch Gen Psychiatry 63, 168-174.
49. Mayeux, R., and Hyslop, P. S. (2008). Alzheimer's disease: advances in trafficking Lancet Neurol 7, 2-3.
50. Farrer, L. A., Cupples, L. A., Haines, J. L., Hyman, B., Kukull, W. A., Mayeux, R., Myers, R. H., Pericak-Vance, M. A., Risch, N., and van Duijn, C. M. (1997). Effects of age, sex, and ethnicity on the association between apolipoprotein E genotype and Alzheimer disease. A meta-analysis. APOE and Alzheimer Disease Meta Analysis Consortium. JAMA 278, 1349-1356.
51. Israel M A, Yuan S H, Bardy C, Reyna S M, Mu Y, Herrera C, Hefferan M P, Van Gorp S, Nazor K L, Boscolo F S, Carson C T, Laurent L C, Marsala M, Gage F H, Remes A M, Koo E H, Goldstein L S (2012). Probing sporadic and familial Alzheimer's disease using induced pluripotent stem cells, Nature, 482(7384):216-20.

1. A method of treating, preventing or delaying the onset of sporadic Alzheimer's Disease in a subject, the method comprising administering to the subject a therapeutically effective amount of an antibody that binds to the APOE4 protein, thereby treating or preventing the Alzheimer's Disease.

2. A method of treating, preventing or delaying the onset of sporadic Alzheimer's Disease in a subject, the method comprising administering to the subject a therapeutically effective amount of a peptide or a peptidomimetic that binds to the APOE4 protein, thereby treating or preventing the Alzheimer's Disease.

3. A method of treating, preventing or delaying the onset of sporadic Alzheimer's Disease in a subject, the method comprising administering to the subject a therapeutically effective amount of an antisense RNA, or a siRNA, that inhibits expression of the gene that encodes the APOE4 protein, thereby treating or preventing the Alzheimer's Disease.

4. The method of claim 1, 2 or 3, wherein the subject is heterozygous for the APOE4 allele.

5. The method of claim 1, 2 or 3, wherein the subject is homozygous for the APOE4 allele.

6. The method of any one of claims 1 to 5, wherein an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

7. The method of claim 1, 4, 5 or 6, wherein the antibody is a monoclonal antibody or a polyclonal antibody.

8. The method of claim 1, 4, 5, 6 or 7, wherein the APOE4 protein comprises SEQ ID NO:1.

9. The method of claim 3, 4, 5 or 6, wherein the antisense RNA or the siRNA binds to a human nucleic acid sequence comprising SEQ ID NO: 2.

10. The method of any one of claim 1, 3, 4, 5, 6, 7 or 8, wherein the treating or preventing comprises reducing the levels of Aβ40, Aβ42, sAPPβ, or any combination thereof, in the subject, as compared to the levels of Aβ40, Aβ42, and sAPPβ in the subject prior to administration of the antibody.

11. The method of any one of claim 2, 3, 4, 5 or 6, wherein the treating or preventing comprises reducing the levels of Aβ40, Aβ42, sAPPβ, or any combination thereof, in the subject, as compared to the levels of Aβ40, Aβ42, and sAPPβ in the subject prior to administration of the peptide or peptidomimetic.

12. The method of any one of claim 3, 4, 5, 6 or 9, wherein the treating or preventing comprises reducing the levels of Aβ40, Aβ42, sAPPβ, or any combination thereof, in the subject, as compared to the levels of Aβ40, Aβ42, and sAPPβ in the subject prior to administration of the antisense RNA or the siRNA.

13. The method of any one of claims 6 to 12, wherein the levels of Aβ40, Aβ42, and sAPPβ in the subject are measured in the cerebro-spinal fluid of the subject.

14. A method of treating, preventing or delaying the onset of sporadic Alzheimer's Disease in a subject, the method comprising administering to the subject a therapeutically effective amount of APOE3 protein, thereby treating or preventing the Alzheimer's Disease.

15. The method of claim 14, wherein the subject is heterozygous for the APOE4 allele

16. The method of claim 14, wherein the subject is homozygous for the APOE4 allele.

17. The method of claim 14, 15, or 16, wherein an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

18. The method of claim 14, 15, 16 or 17, wherein the APOE3 protein is delivered to a cell of the subject through viral-mediated delivery.

19. The method of any one of claims 14 to 18, wherein the APOE3 protein comprises SEQ ID NO:3.

20. The method of any one of claims 14 to 19, wherein the treating or preventing comprises reducing the levels of Aβ40, Aβ42, and sAPPβ, or any combination thereof, in the subject, as compared to the levels of Aβ40, Aβ42, and sAPPβ in the subject prior to administration of the APOE3 protein.

21. The method of claim 17, 18, 19, or 20, wherein the levels of Aβ40, Aβ42, and sAPPβ in the subject are measured in the cerebro-spinal fluid of the subject.

22. A method of treating, preventing or delaying the onset of sporadic Alzheimer's Disease in a subject, the method comprising administering to the subject a therapeutically effective amount of an antibody that binds to the APOE2, APOE3 or APOE4 protein, or any combination thereof, thereby treating or preventing the Alzheimer's Disease.

23. The method of claim 22, wherein the subject is heterozygous for the APOE4 allele or the APOE2 allele.

24. The method of claim 22, wherein the subject is homozygous for the APOE4 allele or the APOE2 allele.

25. The method of claim 22, 23 or 24, wherein the APOE4 protein comprises SEQ ID NO:1.

26. The method of claim 22, 23 or 24, wherein the APOE3 protein comprises SEQ ID NO:3.

27. The method of any one of claims 22 to 26, wherein an indicator of sporadic Alzheimer's Disease is increased levels of Aβ40, increased levels of Aβ42, increased levels of sAPPβ, or any combination thereof, in the subject, as compared to a subject that does not have sporadic Alzheimer's Disease.

28. The method of any one of claims 22 to 27, wherein the antibody is a monoclonal antibody or a polyclonal antibody.

29. The method of any one of claims 22 to 28, wherein the treating or preventing comprises reducing the levels of Aβ40, Aβ42, sAPPβ, or any combination thereof, in the subject, as compared to the levels of Aβ40, Aβ42, and sAPPβ in the subject prior to administration of the antibody.

30. The method of any one of claims 27 to 29, wherein the levels of Aβ40, Aβ42, and sAPPβ in the subject are measured in the cerebro-spinal fluid of the subject.
